(19) United States
(12) Patent Application Publication
(10) Pub. No.: US 2023/0149536 A1
(43) Pub. Date: May 18, 2023

(54) COMPOSITIONS FOR TREATING AND/OR PREVENTING CORONAVIRUS INFECTIONS

(71) Applicants: VYRIAD, INC., Rochester, MN (US); REGENERON PHARMACEUTICALS, INC., Tarrytown, NY (US)

(72) Inventors: Stephen J. RUSSELL, Rochester, MN (US); Kah Whye PENG, Rochester, MN (US); Patrycja LECH, Rochester, MN (US); Alina BAUM, Tarrytown, NY (US); Christos KYRATSOUS, Tarrytown, NY (US)

(21) Appl. No.: 17/919,224

(22) PCT Filed: Apr. 19, 2021
(86) PCT No.: PCT/US2021/027943

§ 371 Date: Oct. 14, 2022

Related U.S. Application Data
(60) Provisional application No. 63/012,070, filed on Apr. 17, 2020.
(60) Provisional application No. 63/040,470, filed on Jun. 17, 2020.
(60) Provisional application No. 63/059,325, filed on Jul. 31, 2020.
(60) Provisional application No. 63/065,896, filed on Aug. 14, 2020.
(60) Provisional application No. 63/078,839, filed on Sep. 15, 2020.
(60) Provisional application No. 63/129,081, filed on Dec. 22, 2020.
(60) Provisional application No. 63/151,279, filed on Feb. 19, 2021.

(51) Int. Cl. A61K 39/215; A61P 31/14; C12N 15/86; C07K 14/005

(52) U.S. Cl. CPC A61K 39/215; A61P 31/14; C12N 15/86; C07K 14/005; A61K 2039/542

(57) ABSTRACT

The disclosure provides recombinant vesicular stomatitis vims (VSV) particles, wherein the VSV glycoprotein (G) is replaced by a coronavirus spike (S) glycoprotein, or a fragment or a derivative thereof, as well as compositions, vaccines, kits, and methods for using the recombinant VSV particles. In a specific embodiment, the S glycoprotein is derived from Severe Acute Respiratory Syndrome coronavirus 2 (SARS-CoV-2) and the methods are for the treatment or prevention of a disease or disorder in a subject infected with SARS-CoV-2. In certain embodiments, the disease or disorder is COVID-19.

### CROSS REFERENCE TO RELATED APPLICATIONS

This patent application claims priority to U.S. Provisional Application No. 63/012,070, filed Apr. 17, 2020, U.S. Provisional Application No. 63/040,470, filed Jun. 17, 2020, U.S. Provisional Application No. 63/059,325, filed Jul. 31, 2020, U.S. Provisional Application No. 63/065,896, filed Aug. 14, 2020, U.S. Provisional Application No. 63/078,839, filed Sep. 15, 2020, U.S. Provisional Application No. 63/129,081, filed Dec. 22, 2020, and U.S. Provisional Application No. 63/151,279, filed Feb. 19, 2021, the disclosures of each of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 16, 2021, is named 48835WO_CRF_sequencelisting.txt and is 103,382 bytes in size.

### TECHNICAL FIELD

Described herein are recombinant vesicular stomatitis virus (VSV) particles, wherein the VSV glycoprotein (G) is replaced by a coronavirus spike (S) glycoprotein, or a fragment or a derivative thereof, as well as compositions, vaccines, kits, and methods for using the recombinant VSV particles. In a specific embodiment, the S glycoprotein is derived from Severe Acute Respiratory Syndrome coronavirus 2 (SARS-CoV-2) and the methods are for the treatment or prevention of a disease or disorder in a subject infected with SARS-CoV-2. In certain embodiments, the disease or disorder is COVID-19.

### BACKGROUND

Three coronaviruses are known to cause severe pneumonia in humans: Severe Acute Respiratory Syndrome coronavirus (SARS-CoV or SARS-CoV-1), Middle East Respiratory Syndrome coronavirus (MERS-CoV), and Severe Acute Respiratory Syndrome coronavirus 2 (SARS-CoV-2). SARS-CoV emerged in China in 2002 and spread to five continents infecting over 8,000 people and causing 774 deaths. MERS-CoV emerged in 2012 in the Arabian Peninsula infecting almost 2,500 people and causing 858 deaths in 27 countries. In December 2019, a new coronavirus emerged in Wuhan, China and caused an acute respiratory disease now known as coronavirus disease 2019 (COVID-19) (Zhou et al., Nature, published online Feb. 3, 2020; available at doi.org/10.1038/s41586-020-2012-7; Zhu et al., New Engl J Med, 2020, 382:727-733). COVID-19 symptoms include fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock and death in severe cases. The virus causing COVID-19 was identified to be related to SARS-CoV and thus was named SARS-CoV-2 (also sometimes referenced as nCov-2019, Wuhan coronavirus, or SARS nCoV19). SARS-CoV-2 is associated with an ongoing world-wide outbreak of atypical pneumonia that has affected over 1.7 million people and killed more than 109,000 people in at least 177 countries as of Apr. 12, 2020. Because of the rapid increase in number of cases worldwide spread, the World Health Organization has declared COVID-19 a pandemic. Many of the patients who develop COVID-19 have mild upper respiratory symptoms, but some (especially older people and people with underlying medical conditions such as chronic lung disease, asthma, heart conditions, diabetes, immunocompromised patients, etc.) develop severe disease (Wölfel et al., Nature, published online on Apr. 1, 2020, available at doi.org/10.1038/s41586-020-2196-x). SARS-CoV-2 is highly contagious and can be spread by asymptomatic carriers. Health care workers are particularly vulnerable to being infected by SARS-CoV-2 when treating patients with COVID-19.

Coronavirus entry into host cells is mediated by the transmembrane spike (S) glycoprotein, which is the main target of anti-viral neutralizing antibodies and is the focus of therapeutic and vaccine design. S glycoprotein forms homotrimers protruding from the viral surface. S glycoprotein comprises two functional subunits responsible for binding to the host cell receptor (S1 subunit) and fusion of the viral and cellular membranes (S2 subunit). For many coronaviruses, including SARS-CoV and SARS-CoV-2, S glycoprotein is cleaved at the boundary between the 51 and S2 subunits, which remain non-covalently bound in the prefusion conformation. The distal S1 subunit comprises the receptor-binding domain(s) (RBD) and contributes to stabilization of the prefusion state of the membrane-anchored S2 subunit that contains the fusion machinery. The S glycoprotein is further cleaved by host proteases at the ST site located immediately upstream of the fusion peptide. This cleavage has been proposed to activate the protein for membrane fusion via conformational changes. Walls et al., Cell, published online Mar. 9, 2020; available at doi.org/10.1016/j.cell.2020.02.058.

SARS-CoV and SARS-CoV-2 can interact directly with angiotensin-converting enzyme 2 (ACE2) to enter target cells, wherein the cellular serine protease TMPRSS2 may prime the S protein priming (Hoffmann et al., Cell, 2020, 181:1-10; available at doi.org/10.1016/j.cell.2020.02.052). SARS-CoV-S and SARS-CoV-2-S share 76% amino acid identity. The receptor binding domain (RBD) in the S glycoprotein is the most variable part of the coronavirus genome. Six RBD amino acids have been shown to be critical for binding to ACE2 receptors and for determining the host range of SARS-CoV-like viruses. They are Y442, L472, N479, D480, T487 and Y4911 in SARS-CoV, which correspond to L455, F486, Q493, S494, N501 and Y505 in SARS-CoV-2 (Andersen et al., Nature Medicine, 2020).

Given the insidious nature of SARS-CoV-2, development of an effective immunogenic composition or vaccine that can treat or prevent a disease or disorder in a subject infected with SARS-CoV-2, such as COVID-19, is highly needed not only to combat an ongoing infection but also to prevent the continuing health threat posed by the virus to those that have not yet been exposed or have not develop lasting immunity to the virus. No commercially available treatments or vaccines have been developed to date.

Studies show that vescisular stomatitis virus (VSV) has potential as a high level expression vector capable of incorporating foreign proteins into its viral envelope (Schnell, et al., 1996 J. Virol. 70, 2318-2323; Schnell, et al., 1996 Proc. Natl. Acad. Sci. USA 93, 11359-11365). VSV is able to cause an extremely rapid cytopathic infection in most animal cells, including human T cells in culture, while normally remaining non-pathogenic in humans (See e.g., Wagner and Rose, 1996). VSV has a non-segmented, negative-strand RNA genome that is transcribed in the cytoplasm of infected cells by the viral RNA polymerase to generate five mRNAs encoding the five structural proteins. Only VSV glycoprotein (G) is present in the viral membrane, wherein it is anchors at the cell surface to catalyzes fusion of the viral membrane with the cellular membrane (Florkiewicz and Rose, 1984). Foreign membrane proteins such as coronavirus spike (S) glycoprotein, or fragments or a derivatives thereof, and other viral proteins can be expressed at very high levels from the genome of recombinant VSVs and these molecules are then incorporated at high levels into the viral membrane along with or in place of VSV's G protein (Schnell, et al., 1996 Proc. Natl. Acad. Sci. USA 93, 11359-11365).

Importantly, not all antibodies produced during an immune response are neutralizing, i.e., are able to interfere with the ability of the virus to infect a cell. Some antibodies can bind specifically to the virus, but do not interfere with its infectivity, because, for example, they might not bind at the right place. While such antibodies can be important to flag the virus for immune cells, the key to an effective treatment or vaccine is the development of neutralizing antibodies that can neutralize the biological effects of the antigen without a need for immune cells. Thus, there exists a great need for an effective immunogenic and/or antigenic composition or vaccine for SARS-CoV-2 and other coronaviruses which can induce the formation of protective immunity.

### SUMMARY

As specified in the Background section, above, there is a great need for the development of an effective immunogenic and/or antigenic composition or vaccine for SARS-CoV-2 and other coronaviruses. The present disclosure addresses these and other needs. The present disclosure is based on the realization that the effective immunogenic and/or antigenic composition or vaccine should specifically induce the formation of neutralizing antibodies. The present disclosure provides recombinant vesicular stomatitis virus (VSV) particles expressing coronavirus proteins that can be administered as an immunogenic and/or antigenic composition or vaccine to induce the formation of coronavirus neutralizing antibodies resulting in protective immunity. In certain instances, the VSV glycoprotein (G) is replaced by a coronavirus spike (S) glycoprotein or a fragment or a derivative thereof. In a specific embodiment, the S glycoprotein is derived from Severe Acute Respiratory Syndrome coronavirus 2 (SARS-CoV-2) and the methods are used to induce the formation of SARS-CoV-2 neutralizing antibodies. In certain embodiments, the methods are used to induce a protective immune response against SARS-CoV-2.

In one aspect, the invention provides a recombinant rhabdovirus particle comprising a rhabdovirus genome lacking a functional rhabdovirus glycoprotein (G) gene, wherein the recombinant rhabdovirus particle comprises a polynucleotide sequence encoding at least one Severe Acute Respiratory Syndrome coronavirus 2 (SARS-CoV-2) spike (S) glycoprotein or fragment or derivative thereof.

In another aspect, the invention provides a recombinant vesiculovirus particle comprising a vesiculovirus genome lacking a functional vesiculovirus G gene, wherein the recombinant vesiculovirus particle comprises a polynucleotide sequence encoding at least one SARS-CoV-2 S glycoprotein or fragment or derivative thereof.

In another aspect, the invention provides a recombinant vesicular stomatitis virus (VSV) particle comprising a VSV genome lacking a functional VSV G gene, wherein the recombinant VSV particle comprises a polynucleotide sequence encoding at least one SARS-CoV-2 S glycoprotein or fragment or derivative thereof.

In certain embodiments, the recombinant virus particle (i.e., the recombinant rhabdovirus particle, the recombinant vesiculovirus particle, or recombinant VSV particle) genome comprises the polynucleotide sequence encoding the at least one SARS-CoV-2 S glycoprotein or fragment or derivative thereof. In certain embodiments, the polynucleotide sequence encoding the at least one SARS-CoV-2 S glycoprotein or fragment or derivative thereof is not part of the virus genome. In certain embodiments, the recombinant virus particle comprises or expresses the SARS-CoV-2 S glycoprotein or fragment or derivative thereof on the viral envelope. In certain embodiments, the SARS-CoV-2 S glycoprotein or fragment or derivative thereof is immunogenic and/or antigenic.

In certain embodiments, the recombinant virus particle the recombinant virus particle is replication-competent. In certain embodiments, the recombinant virus particle the recombinant virus particle is replication-deficient.

In certain embodiments, the SARS-CoV-2 S glycoprotein or fragment or derivative thereof is capable of targeting a receptor on a host cell. In certain embodiments, targeting of the receptor results in the recombinant virus infecting the host cell. In certain embodiments, the receptor is an angiotensin converting enzyme 2 (ACE2).

In certain aspects, the SARS-CoV-2 S glycoprotein comprises the amino acid sequence of SEQ ID NO: 1 or an amino acid sequence that has at least 80% sequence identity to SEQ ID NO: 1. In certain embodiments, the polynucleotide sequence encoding the SARS-CoV-2 S glycoprotein comprises SEQ ID NO: 2 or a polynucleotide sequence that has at least 80% sequence identity to SEQ ID NO: 2.

In certain aspects, the recombinant virus particle comprises a fragment of the SARS-CoV-2 S glycoprotein. In certain embodiments, the virus genome encodes the fragment of the SARS-CoV-2 S glycoprotein. In certain aspects, the virus genome encodes a fragment of the SARS-CoV-2 S glycoprotein. In certain embodiments, the fragment comprises an S1 subunit, S2 subunit, and/or receptor-binding domain (RBD), or fragments or derivatives thereof, of the SARS-CoV-2 S glycoprotein. In certain embodiments, fragment comprises an RBD or an amino acid sequence that has at least 80% sequence identity to the RBD. In certain embodiments, the fragment consists of the RBD.

In certain embodiments, the fragment is a C-terminally truncated SARS-CoV-2 S glycoprotein. In certain embodiments, the C-terminally truncated SARS-CoV-2 S glycoprotein comprises a deletion of one to 30 amino acids from the C-terminus of the SARS-CoV-2 S glycoprotein. In certain embodiments, the C-terminally truncated SARS-CoV-2 S glycoprotein comprises a 19 amino acid deletion from the C-terminus of the of SARS-CoV-2 S glycoprotein. In certain embodiments, the C-terminally truncated SARS-CoV-2 S glycoprotein comprises or consists of the amino acid sequence of SEQ ID NO: 3 or an amino acid sequence that has at least 80% sequence identity to SEQ ID NO: 3. In certain embodiments, the polynucleotide sequence encoding the C-terminally truncated SARS-CoV-2 S glycoprotein comprises or consists of SEQ ID NO: 4 or a polynucleotide sequence that has at least 80% sequence identity to SEQ ID NO: 4. In certain embodiments, the C-terminally truncated SARS-CoV-2 S glycoprotein comprises or consists of the amino acid sequence of SEQ ID NO: 20 or an amino acid sequence that has at least 80% sequence identity to SEQ ID NO: 20. In certain embodiments, the polynucleotide sequence encoding the C-terminally truncated SARS-CoV-2 S glycoprotein comprises or consists of SEQ ID NO: 21 or a polynucleotide sequence that has at least 80% sequence identity to SEQ ID NO: 21. In certain embodiments, the C-terminally truncated SARS-CoV-2 S glycoprotein comprises or consists of the amino acid sequence of SEQ ID NO: 22 or an amino acid sequence that has at least 80% sequence identity to SEQ ID NO: 22.

In certain aspects, the recombinant virus particle comprises a derivative of the SARS-CoV-2 S glycoprotein, wherein the derivative is a SARS-CoV-2 S fusion protein. In certain embodiments, SARS-CoV-2 S fusion protein is a fusion between a SARS-CoV-2 S glycoprotein, or fragment or derivative thereof, and a protein the enables viral entry. In certain embodiments, the protein that enables viral entry is a non-SARS-CoV-2 fusogen or fragment or derivative thereof. In certain embodiments, the fusogen is a VSV glycoprotein (G) protein or fragment or derivative thereof. In certain embodiments, the fragment of the VSV G protein is a VSV G protein cytoplasmic tail. In certain embodiments, the VSV G protein cytoplasmic tail comprises SEQ ID NO: 15 or an amino acid sequence that has at least 80% sequence identity to SEQ ID NO: 15. In certain embodiments, the SARS-CoV-2 S fusion protein comprises the amino acid sequence of SEQ ID NO: 5 or an amino acid sequence that has at least 80% sequence identity to SEQ ID NO: 5. In certain embodiments, the polynucleotide sequence encoding the SARS-CoV-2 S fusion protein comprises SEQ ID NO: 6 or a polynucleotide sequence that has at least 80% sequence identity to SEQ ID NO: 6.

In certain embodiments, the recombinant virus particle comprises the fragment or derivative of the SARS-CoV-2 S glycoprotein, wherein the fragment or derivative of the SARS-CoV-2 S glycoprotein results in a more fusogenic recombinant virus particle as compared to a comparable recombinant virus particle comprising a full-length wild-type SARS-CoV-2 spike glycoprotein. In certain embodiments, the fragment or derivative of the SARS-CoV-2 S glycoprotein and the full-length wild-type SARS-CoV-2 spike glycoprotein are inserted into the same position in the virus genome of the respective virus particles.

In certain embodiments, the polynucleotide that encodes the at least one SARS-CoV-2 S protein or fragment or derivative thereof is inserted within the virus G gene. In certain embodiments, the virus G gene is replaced by a polynucleotide encoding the at least one SARS-CoV-2 S protein or fragment or derivative thereof. In certain embodiments, the polynucleotide that encodes the at least one SARS-CoV-2 S protein or fragment or derivative thereof is inserted within a non-essential portion of the recombinant virus genome.

In certain embodiments, the genome of the recombinant VSV particle comprises genes encoding VSV nucleoprotein (N), VSV phosphoprotein (P), and VSV large protein (L) proteins, or functional fragments or derivatives thereof.

In certain embodiments, the genome of the recombinant VSV particle encodes a wild-type VSV matrix (M) protein. In certain embodiments, the VSV M protein comprises the amino acid sequence of SEQ ID NO: 9 or an amino acid sequence that has at least 80% sequence identity to SEQ ID NO: 9. In certain embodiments, the polynucleotide sequence encoding the VSV M protein comprises SEQ ID NO: 10 or a polynucleotide sequence that has at least 80% sequence identity to SEQ ID NO: 10.

In certain embodiments, the genome of the recombinant VSV particle encodes a mutant VSV M protein. In certain embodiments, the mutant VSV M protein comprises a mutation at methionine (M) 51. In certain embodiments, the mutation is from methionine (M) to arginine (R). In certain embodiments, the mutant VSV M protein comprises the amino acid sequence of SEQ ID NO: 7 or an amino acid sequence that has at least 80% sequence identity to SEQ ID NO: 7. In certain embodiments, the polynucleotide sequence encoding the mutant VSV M protein comprises SEQ ID NO: 8 or a polynucleotide sequence that has at least 80% sequence identity to SEQ ID NO: 8. In certain embodiments, the mutant VSV M protein comprises a deletion at methionine (M) 51.

In another aspect, the invention provides a polynucleic acid comprising a polynucleotide sequence encoding a rhabdovirus nucleoprotein (N), a rhabdovirus phosphoprotein (P), and a rhabdovirus large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant rhabdovirus particle.

In one aspect, the invention provides polynucleic acid comprising a polynucleotide sequence encoding a vesiculovirus nucleoprotein (N), a vesiculovirus phosphoprotein (P), and a vesiculovirus large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant vesiculovirus particle.

In one aspect, the invention provides polynucleic acid comprising a polynucleotide sequence encoding vesicular stomatitis virus (VSV) nucleoprotein (N), a VSV phosphoprotein (P), and a VSV large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant VSV particle.

In certain embodiments, the SARS-CoV-2 S glycoprotein or fragment or derivative thereof is immunogenic and/or antigenic.

In certain embodiments, the SARS-CoV-2 S glycoprotein or fragment or derivative thereof is capable of targeting a SARS-CoV-2 spike protein receptor on a host cell comprising. In certain embodiments, the targeting of the receptor results in the recombinant virus particle infecting the host cell. In certain embodiments, the receptor is an angiotensin converting enzyme 2 (ACE2). In certain embodiments, the SARS-CoV-2 S glycoprotein comprises the amino acid sequence of SEQ ID NO: 1 or an amino acid sequence that has at least 80% sequence identity to SEQ ID NO: 1. In certain embodiments, the polynucleotide sequence encoding the SARS-CoV-2 S glycoprotein comprises SEQ ID NO: 2 or a polynucleotide sequence that has at least 80% sequence identity to SEQ ID NO: 2.

In certain embodiments, the polynucleotide sequence encodes a fragment of the SARS-CoV-2 S glycoprotein. In certain embodiments, the fragment comprises an S1 subunit, S2 subunit, and/or receptor-binding domain (RBD), or fragments or derivatives thereof, of the SARS-CoV-2 S glycoprotein. In certain embodiments, the fragment comprises an RBD or an amino acid sequence that has at least 80% sequence identity to the RBD derivatives thereof. In certain embodiments, the fragment consists of the RBD.

In certain embodiments, the fragment is a C-terminally truncated SARS-CoV-2 S glycoprotein. In certain embodiments, the C-terminally truncated SARS-CoV-2 S glycoprotein comprises a deletion of one to 30 amino acids from the C-terminus of the SARS-CoV-2 S glycoprotein. In certain embodiments, the C-terminally truncated SARS-CoV-2 S glycoprotein comprises a 19 amino acid deletion from the C-terminus of the of SARS-CoV-2 S glycoprotein. In certain embodiments, the C-terminally truncated SARS-CoV-2 S glycoprotein comprises the amino acid sequence of SEQ ID NO: 3 or an amino acid sequence that has at least 80% sequence identity to SEQ ID NO: 3. In certain embodiments, the polynucleotide sequence encoding the C-terminally truncated SARS-CoV-2 S glycoprotein comprises SEQ ID NO: 4 or a polynucleotide sequence that has at least 80% sequence identity to SEQ ID NO: 4. In certain embodiments, the C-terminally truncated SARS-CoV-2 S glycoprotein comprises the amino acid sequence of SEQ ID NO: 20 or an amino acid sequence that has at least 80% sequence identity to SEQ ID NO: 20. In certain embodiments, the polynucleotide sequence encoding the C-terminally truncated SARS-CoV-2 S glycoprotein comprises SEQ ID NO: 21 or a polynucleotide sequence that has at least 80% sequence identity to SEQ ID NO: 21. In certain embodiments, the C-terminally truncated SARS-CoV-2 S glycoprotein comprises the amino acid sequence of SEQ ID NO: 22 or an amino acid sequence that has at least 80% sequence identity to SEQ ID NO: 22.

In certain embodiments, polynucleotide sequence encodes a derivative of the SARS-CoV-2 S glycoprotein, wherein the derivative is a SARS-CoV-2 S fusion protein. In certain embodiments, the SARS-CoV-2 S fusion protein is a fusion between a SARS-CoV-2 S glycoprotein, or fragment or derivative thereof, and a non-SARS-CoV-2 fusogen or fragment or derivative thereof. In certain embodiments, the fusogen is a VSV glycoprotein (G) protein or fragment or derivative thereof. In certain embodiments, the VSV G protein fragment is a VSV G protein cytoplasmic tail. In certain embodiments, the SARS-CoV-2 S fusion protein comprises the amino acid sequence of SEQ ID NO: 5 or an amino acid sequence that has at least 80% sequence identity to SEQ ID NO: 5.3′ to the SARS-CoV-2 S glycoprotein or fragment or derivative thereof. In certain embodiments, the polynucleotide sequence encoding the SARS-CoV-2 S fusion protein comprises SEQ ID NO: 6 or a polynucleotide sequence that has at least 80% sequence identity to SEQ ID NO: 6.

In certain embodiments, the polynucleotide sequence further comprises a Kozak sequence polynucleotide. In certain embodiments, the Kozak sequence is a wild-type Kozak sequence. In certain embodiments, the wild-type Kozak sequence comprises SEQ ID NO: 11 or a derivative thereof. In certain embodiments, the Kozak sequence is an optimized Kozak sequence. In certain embodiments, the optimized Kozak sequence comprises SEQ ID NO: 12 or a derivative thereof.

In certain embodiments, polynucleotide sequence further encodes a wild-type VSV matrix (M) protein. In certain embodiments, VSV M protein comprises the amino acid sequence of SEQ ID NO: 9 or an amino acid sequence that has at least 80% sequence identity to SEQ ID NO: 9. In certain embodiments, the polynucleotide sequence encoding the VSV M protein comprises SEQ ID NO: 10 or a polynucleotide sequence that has at least 80% sequence identity to SEQ ID NO: 10. In certain embodiments, the polynucleotide sequence further encodes a mutant VSV M protein. In certain embodiments, the mutant VSV M protein comprises a mutation at methionine (M) 51. In certain embodiments, the mutation is from methionine (M) to arginine (R). In certain embodiments, the mutant VSV M protein comprises the amino acid sequence of SEQ ID NO: 7 or an amino acid sequence that has at least 80% sequence identity to SEQ ID NO: 7. In certain embodiments, the mutant VSV M protein comprises SEQ ID NO: 8 or a polynucleotide sequence that has at least 80% sequence identity to SEQ ID NO: 8. In certain embodiments, the mutant VSV M protein comprises at a deletion at methionine (M) 51.

In certain embodiments, the polynucleotide sequence lacks a functional G protein gene. In certain embodiments, the polynucleotide sequence encoding the SARS-CoV-2 S glycoprotein or fragment or derivative thereof is inserted within the virus G protein gene. In certain embodiments, the virus G protein gene is replaced by the polynucleotide sequence encoding the SARS-CoV-2 S glycoprotein or fragment or derivative thereof. In certain embodiments, the polynucleotide sequence encoding the SARS-CoV-2 S glycoprotein or fragment or derivative thereof is inserted within a non-essential portion of the recombinant virus genome.

In another aspect, the invention provides a composition comprising the polynucleotide as described herein and a carrier and/or excipient.

In another aspect, the invention provides a replication-competent recombinant vesicular stomatitis virus (VSV) particle comprising the polynucleotide as described herein.

In another aspect, the invention provides a host cell comprising the recombinant virus particle as described herein.

In another aspect, the invention provides a composition comprising the recombinant virus particle as described herein and a carrier and/or excipient.

In another aspect, the invention provides a pharmaceutical composition comprising the recombinant virus particle as described herein and a pharmaceutically acceptable carrier and/or excipient.

In another aspect, the invention provides a pharmaceutical composition comprising an inactivated recombinant virus particle as described herein and a pharmaceutically acceptable carrier and/or excipient.

In another aspect, the invention provides an immunogenic composition comprising an amount of the recombinant virus particle as described herein effective to induce an immune response against a SARS-CoV-2 and a pharmaceutically acceptable carrier and/or excipient.

In another aspect, the invention provides an immunogenic composition comprising an amount of the recombinant virus particle as described herein effective to induce the formation of neutralizing antibodies against a SARS-CoV-2 and a pharmaceutically acceptable carrier and/or excipient.

In another aspect, the invention provides a vaccine formulation comprising an amount of the recombinant virus particle as described herein effective to induce an immune response against a SARS-CoV-2 and a pharmaceutically acceptable carrier and/or excipient.

In another aspect, the invention provides a vaccine formulation comprising an amount of the recombinant virus particle as described herein effective to induce the formation of neutralizing antibodies against a SARS-CoV-2 and a pharmaceutically acceptable carrier and/or excipient.

In certain embodiments, the invention provides a vaccine formulation providing stability of the pharmaceutical composition at 4° C. In certain embodiments, the vaccine formulation increases the amount of time the recombinant virus particles as described herein remain viable at 4° C. In certain embodiments, the vaccine formulation is stable after at least three freeze/thaw cycles. In certain embodiments, the vaccine formulation allows the recombinant virus particles as described herein to remain viable after three freeze/thaw cycles.

In another aspect, the invention provides for a vaccine formation that increases the time the pharmaceutical composition is in contact with mucous membranes. In certain embodiments, the invention provides for an orally administered vaccine formation that increases the time the pharmaceutical composition is in contact with mucous membranes.

In certain embodiments, the vaccine composition and/or formulation comprises 50 mM Tris and 2 mM MgCl₂ and is at pH 7.4. In certain embodiments, the vaccine composition and/or formulation comprises a carrier and/or excipient that comprises at least one of methylcellulose, monosodium glutamate, human serum albumin, fetal bovine serum, trehalose, alginate, guar gum, or MUCOLOX™. In certain embodiments, the vaccine composition and/or formulation comprises 50 mM Tris HCL (pH 7.4), 2 mM MgCl₂, 10% Trehalose, and 0.25% Human Serum Albumin.

In another aspect, the invention provides a method of treating or preventing a disease or disorder in a subject comprising administering to the subject an amount of the recombinant virus particle as described herein, the pharmaceutical composition as described herein, the immunogenic composition as described herein, or the vaccine formulation as described herein. In certain embodiments, the disease or disorder is COVID-19.

In another aspect, the invention provides a method of treating or preventing a disease or disorder in a subject comprising administering to the subject an amount of the recombinant virus particle as described herein, the pharmaceutical composition as described herein, the immunogenic composition as described herein, or the vaccine formulation as described herein effective to induce an immune response against a SARS-CoV-2. In certain embodiments, the disease or disorder is COVID-19.

In another aspect, the invention provides a method of treating or preventing a disease or disorder in a subject comprising administering to the subject an amount of the recombinant virus particle as described herein, the pharmaceutical composition as described herein, the immunogenic composition as described herein, or the vaccine formulation as described herein effective to induce the formation of neutralizing antibodies against a SARS-CoV-2. In certain embodiments, the disease or disorder is COVID-19.

In another aspect, the invention provides a method of treating a subject infected with a SARS-CoV-2 comprising administering to the subject an amount of the recombinant virus particle as described herein, the pharmaceutical composition as described herein, the vaccine formulation as described herein, or the vaccine formulation as described herein effective to target the subject's cells harboring the SARS-CoV-2.

In another aspect, the invention provides a method of treating or preventing a disease or disorder in a subject comprising administering to the subject a boosting dose of the recombinant virus particle as described herein, the pharmaceutical composition as described herein, the immunogenic composition as described herein, or the vaccine formulation as described herein. In certain embodiments, the disease or disorder is COVID-19. In certain embodiments, the boosting dose is administered orally.

In another aspect, the invention provides a method of treating or preventing a disease or disorder in a subject comprising administering to the subject a boosting dose of the recombinant virus particle as described herein, the pharmaceutical composition as described herein, the immunogenic composition as described herein, or the vaccine formulation as described herein effective to induce the formation of neutralizing antibodies against a SARS-CoV-2. In certain embodiments, the disease or disorder is COVID-19. In certain embodiments, the boosting dose is administered orally.

In another aspect, the invention provides a method of treating a subject infected with a SARS-CoV-2 comprising administering to the subject a boosting dose of the recombinant virus particle as described herein, the pharmaceutical composition as described herein, the vaccine formulation as described herein, or the vaccine formulation as described herein effective to target the subject's cells harboring the SARS-CoV-2. In certain embodiments, the boosting dose is administered orally.

In certain embodiments of the methods described herein, the subject is human.

In another aspect, the invention provides a kit comprising an amount of the recombinant virus particle as described herein, the pharmaceutical composition as described herein, the vaccine formulation as described herein, or the vaccine formulation as described herein and, optionally, instructions.

In another aspect, the invention provides a kit comprising an amount of the recombinant virus particle as described herein, the pharmaceutical composition as described herein, the vaccine formulation as described herein, or the vaccine formulation as described herein effective to induce an immune response against the SARS-CoV-2 and, optionally, instructions.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 3 or an amino acid sequence that has at least 80% sequence identity to SEQ ID NO: 3, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 3 or an amino acid sequence that has at least 80% sequence identity to SEQ ID NO: 3, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a derivative of a SARS-CoV-2 spike (S) glycoprotein comprising the amino acid sequence of SEQ ID NO: 5 or an amino acid sequence that has at least 80% sequence identity to SEQ ID NO: 5, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a derivative of a SARS-CoV-2 spike (S) glycoprotein comprising the amino acid sequence of SEQ ID NO: 5 or an amino acid sequence that has at least 80% sequence identity to SEQ ID NO: 5, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a fragment of a SARS-CoV-2 spike (S) glycoprotein comprising the polynucleotide sequence of SEQ ID NO: 4 or an polynucleotide sequence that has at least 80% sequence identity to SEQ ID NO: 4, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a fragment of a SARS-CoV-2 spike (S) glycoprotein comprising the polynucleotide sequence of SEQ ID NO: 4 or an polynucleotide sequence that has at least 80% sequence identity to SEQ ID NO: 4, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a derivative of a SARS-CoV-2 spike (S) glycoprotein comprising the polynucleotide sequence of SEQ ID NO: 6 or an polynucleotide sequence that has at least 80% sequence identity to SEQ ID NO: 6, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a derivative of a SARS-CoV-2 spike (S) glycoprotein comprising the polynucleotide sequence of SEQ ID NO: 6 or an polynucleotide sequence that has at least 80% sequence identity to SEQ ID NO: 6, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 20 or an amino acid sequence that has at least 80% sequence identity to SEQ ID NO: 20, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 20 or an amino acid sequence that has at least 80% sequence identity to SEQ ID NO: 20, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a fragment of a SARS-CoV-2 spike (S) glycoprotein comprising the polynucleotide sequence of SEQ ID NO: 21 or an polynucleotide sequence that has at least 80% sequence identity to SEQ ID NO: 21, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a fragment of a SARS-CoV-2 spike (S) glycoprotein comprising the polynucleotide sequence of SEQ ID NO: 21 or an polynucleotide sequence that has at least 80% sequence identity to SEQ ID NO: 21, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 22 or an amino acid sequence that has at least 80% sequence identity to SEQ ID NO: 22, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In another aspect, the invention provides a replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 22 or an amino acid sequence that has at least 80% sequence identity to SEQ ID NO: 22, wherein the polynucleotide sequence encoding the SARS-CoV-2 S glycoprotein replaces the VSV G gene and optionally further comprises an optimized Kozak sequence of SEQ ID NO: 12 3′ to the SARS-CoV-2 S glycoprotein fragment polynucleotide.

In yet another aspect, provided herein is a recombinant virus particle, wherein the recombinant virus particle is a recombinant vesiculovirus particle comprising a vesiculovirus genome lacking a functional vesiculovirus glycoprotein G gene, and further wherein the recombinant virus particle comprises a polynucleotide sequence encoding at least one Severe Acute Respiratory Syndrome coronavirus 2 (SARS-CoV-2) spike (S) glycoprotein or fragment or derivative thereof.

In certain embodiments, the recombinant vesiculovirus particle further comprises a pseudotyped G glycoprotein or fragment or derivative that is derived from a rhabdovirus that is not the recombinant vesiculovirus.

In certain embodiments, the polynucleotide sequence encoding the SARS-CoV-2 S glycoprotein or fragment comprises one or more mutations.

In certain embodiments, the recombinant virus particle is a vaccine.

In certain embodiments, the vaccine is administered orally.

In certain embodiments, the vaccine is administered as a primary vaccination or a boost.

These and other aspects described herein will be apparent to those of ordinary skill in the art in the following description, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts SARS-CoV-2 constructs used in the recombinant VSV particles generated in Example 1 (variants 1-4). In these constructs, the VSV G glycoprotein was substituted by: (1) full length SARS-CoV-2 spike (S) glycoprotein sequence (variant 1; VSV SARS-CoV-2 dG; amino acid sequence SEQ ID NO: 1; codon-optimized polynucleotide sequence SEQ ID NO: 2), (2) SARS-CoV-2 S glycoprotein sequence with a deletion of 19 amino acids KFDEDDSEPVLKGVKLHYT (SEQ ID NO: 14) in the cytoplasmic tail (variant 2; VSV SARS-CoV-2 Δ19CT dG; amino acid sequence SEQ ID NO: 3; codon-optimized polynucleotide sequence SEQ ID NO: 4); (3) SARS-CoV-2 S glycoprotein sequence with a replacement of the S cytoplasmic tail with a VSV G cytoplasmic tail sequence (KLKHTKKRQIYTDIEMNRLGK (SEQ ID NO: 15)) (variant 3; VSV SARS-CoV-2 VSV-G CT dG; amino acid sequence SEQ ID NO: 5; codon-optimized polynucleotide sequence SEQ ID NO: 6); or (4) full length SARS-CoV-2 S glycoprotein sequence but using the wild-type VSV Kozak sequence (cActATG; SEQ ID NO: 11) in place of the optimized Kozak sequence (caccATG; SEQ ID NO: 12) used in the other three constructs (variant 4; VSV SARS-CoV-2 dG; amino acid sequence SEQ ID NO: 1; codon-optimized polynucleotide sequence SEQ ID NO: 2). One set of variant 1-4 constructs was prepared that encoded wild-type VSV matrix (M) protein (amino acid sequence SEQ ID NO: 9; polynucleotide sequence SEQ ID NO: 10). A second set of variant 1-4 constructs was prepared that encoded VSV M protein with the substitution M51R variant M protein (amino acid sequence SEQ ID NO: 7; polynucleotide sequence SEQ ID NO: 8), resulting in VSV attenuation.

FIG. 2 depicts a Western blot showing expression of VSV G, nucleoprotein (N), and M proteins, and SARS-CoV-2 (SARS nCoV19) S glycoprotein in the recombinant VSV-M51R-nCoV19-S Δ19CT (variant 2; VSV SARS-CoV-2 Δ19CT dG) virions. SARS-CoV-2 S Δ19CT glycoprotein produced two bands corresponding to the full-length (180 kDa) and the proteolytically cleaved (75 kDa) glycoprotein. The Western blot shows the presence of VSV N, M and G proteins in the parental VSV-GFP virus and the presence of VSV N and M proteins (but not VSV G glycoprotein) in the variant 2 VSV SARS-CoV-2 Δ19CT dG construct 6 (VSV-M51R-nCoV19-S Δ19CT) virus. The Western blot for variant 2 VSV SARS-CoV-2 Δ19CT dG construct 6 (VSV-M51R-nCoV19-S Δ19CT) virus also shows efficient incorporation of SARS-CoV-2 S Δ19CT glycoprotein in place of the VSV G glycoprotein.

FIG. 3 shows photographs of Vero-αHis cells 18, 21, and 35 hours after being infected (hours post-infection; hpi) with variant 2 VSV SARS-CoV-2 Δ19CT dG construct 6 (VSV-M51R-nCoV19-S Δ19CT) viral particles showing that the recombinant VSV SARS-CoV-2 Δ19CT dG viral particles successfully underwent cell fusion.

FIG. 4A and FIG. 4B depict photographs of a mixture of Vero-DSP-1-Puro and Vero-DSP-2-Puro cells infected with variant 2, VSV SARS-CoV-2 Δ19CT dG construct 6 (VSV-M51R-COVID-SΔ19CT dG) recombinant virus or control mock-infected cells at 16 hours after being infected (hpi) with 4 μg/mL of trypsin added at 4 hpi. A control Vero-DSP1-Puro/Vero-DSP2-Puro cell mixture was infected with the same construct, but not treated with trypsin. Another control Vero-DSP1-PuroNero-DSP2-Puro cell mixture was not infected with the construct (mock) and was either treated with 4 μg/mL of trypsin in OptiMem or not treated with trypsin. FIG. 4B depicts luciferase signal of mixed Vero-DSP1-Puro/Vero-DSP2-Puro detected 22 hours after infection (hpi) with VSV SARS-CoV-2 Δ19CT dG (variant 2).

FIG. 5 depicts an example testing regimen.

FIG. 6 depicts an example testing regimen.

FIG. 7 depicts an example testing regimen.

FIG. 8 depicts an example testing regimen.

FIG. 9A and FIG. 9B depict a neutralizing antibody screen showing the presence of neutralizing antibodies in the non-human primate (NHP) sera for 4 out of the 6 animals evaluated by Day 14. Comparison by each collection interval (Pretest and Days 1, 4, 7, 11, and 14): NHP sera were diluted to the minimum recommended dilution established in the neutralizing antibody assay (1:50 for NHP serum matrix). Diluted samples were incubated with VSV-SARS-CoV-2-S-Δ19CT prior to infecting Vero cell monolayers. The Vero cell monolayer consisted of a mixture of two complimentary variants of a luciferase-based reporter system. Virus-induced cell fusion causes the production of a functional luciferase enzyme, and following incubation with substrate, chemiluminescent signal was read. A reduction of Relative Light Units (RLU) starting at Day 7 (Animal CVAXE-1 and -4) and Day 11 (Animals CVAXE-3 and -5) indicate the presence of neutralizing antibodies. The assay was read at both 24 hours post infection (hpi) (FIG. 9A) and 32 hpi (FIG. 9B).

FIG. 10A and FIG. 10B depict a neutralizing antibody titer at Day 14. NHP sera were diluted starting at the minimum recommended dilution established in the neutralizing antibody assay (1:50 for NHP serum matrix) and further serial diluted 2-fold to a maximum dilution of 1:6400. Diluted samples were incubated with VSV-SARS-CoV-2-S-Δ19CT prior to infecting Vero cell monolayers. The Vero cell monolayer consisted of a mixture of two complimentary variants of a luciferase-based reporter system. Virus-induced cell fusion causes the production of a functional luciferase enzyme, and following incubation with substrate, chemiluminescent signal was read. Resulting RLU for each dilution were fitted to a 4-parameter logistic regression model, and the EC₅₀, meaning the dilution that resulted in the half maximal luciferase signal was determined. The EC₅₀ value serves to provide a measure of the level of neutralizing capacity for each of the Day 14 NHP serum samples. Assay was read at both 24-hours (FIG. 10A) and 32-hours (FIG. 10B) post infection.

FIG. 11 depicts a variant spike glycoprotein for use in the recombinant VSV particles disclosed herein (CPE variant). It is a SARS-CoV-2 S glycoprotein variant sequence, with S247R, D614N and R685Q substitutions and with a deletion of 19 amino acids KFDEDDSEPVLKGVKLHYT (SEQ ID NO: 14) in the cytoplasmic tail (CPE variant 2; SARS-CoV-2 Δ19CT CPE Lytic Variant; amino acid sequence SEQ ID NO: 20; codon-optimized polynucleotide sequence SEQ ID NO: 21). SP, Signal peptide; NTD, N-terminal domain; RBD, Receptor binding domain; SD1, Subdomain1; SD2, Subdomain 2; FP, Fusion peptide; HR1, Heptad Repeat 1; HR2, Heptad Repeat 2; TM, Transmembrane; CT, Cytoplasmic tail; and Δ19, 19 amino acid deletion.

FIG. 12 depicts a Western blot showing expression of VSV G, N, P, and M proteins, and SARS-CoV-2 (SARS nCoV19) S glycoprotein in VSV-SARS2 virions (a recombinant Indiana strain of Vesicular Stomatitis Virus whereby its G glycoprotein is replaced by the spike glycoprotein of SARS-CoV-2 with a deletion of 19 amino acids KFDEDDSEPVLKGVKLHYT (SEQ ID NO: 14)) and VSV-SARS2+VSV-G virions (VSV-SARS2.G, which are VSV-SARS2 virions pseudotyped with the VSV.G glycoprotein).

FIG. 13 depicts the effects of the VSV-SARS2 vaccine administration on animal bodyweight and temperature.

FIG. 14A, FIG. 14B, and FIG. 14C depict anti-SARS-CoV-2 spike antibody titers for IgM (FIG. 14A), IgG (FIG. 14B), and IgA (FIG. 14C) in the non-human primate (NHP) sera by Day 42 (Pretest and Days 1, 4, 7, 11, 14, 21, 28, 35, and 42). Results are depicted as fold change over baseline.

FIG. 15 depicts anti-SARS-CoV-2 spike antibody response to S-trimer IgG in the non-human primate (NHP) sera by Day 70 (Pretest and Days 1, 4, 7, 11, 14, 21, 28, 35, 42, 56, and 70).

FIG. 16 depicts neutralizing antibody activity for all animals from Day 0 through Day 42.

FIG. 17 depicts neutralizing antibody activity measured by a BSL3 clinical isolate of SARS-CoV-2, evaluated by PRNT assay.

FIG. 18 depicts anti-G mediated VSV neutralizing antibodies. Data show the immunogenicity response against vaccine platform.

FIG. 19 shows that T-cell responses to SARS-CoV-2 spike 51 and S2 mega-peptide pools peak at Day 14. T-cell mediated immune response was measured by a Fluoro Spot assay.

FIG. 20 depicts the neutralization of VSV-SARS2 infectivity by anti-SARS-CoV-2 Spike monoclonal antibody and human convalescent serum.

FIG. 21 depicts the stability of VSV-SARS2 and VSV-SARS2.G formulations at 4° C. on days 0, 4, 6, 8, 10, 12, 14 and 20. Virus titer is calculated as the percentage of day 0 titer.

FIG. 22 depicts the stability of VSV-SARS2 formulations at 4° C. on days 0, 6, 14 and 20. Virus titer is calculated as the percentage of day 0 titer.

FIG. 23 depicts the stability of VSV-SARS2 formulations at 4° C. on days 0, 6, 14 and 20. Virus titer is calculated as the percentage of day 0 titer.

FIG. 24 depicts the stability of VSV-SARS2 formulations at 4° C. on days 0, 6, 14 and 20. Virus titer is calculated as the percentage of day 0 titer.

FIG. 25 depicts the stability of VSV-SARS2 formulations after three freeze/thaw cycles.

FIG. 26 depicts the stability of VSV-SARS2 formulations after three freeze/thaw cycles.

FIG. 27 depicts the stability of VSV-SARS2.G formulations after three freeze/thaw cycles.

FIG. 28A and FIG. 28B depict the stability of VSV-SARS2 (FIG. 28A) and VSV-SARS2.G (FIG. 28B) mucoadhesive formulations.

FIG. 29 depicts the stability of VSV-SARS2 mucoadhesive formulations.

FIG. 30A depicts anti-Spike IgG levels relative to pre-dose levels. FIG. 30B depicts luciferase levels relative to pre-dose levels resulting from the neutralizing antibody assay.

FIG. 31 depicts the increase in virus neutralizing units following oral vaccine boost.

FIG. 32 depicts serum IgG binding to SAR-CoV-2 spike trimer evaluated by ELISA.

FIG. 33 depicts detection of spike specific T cell responses. Responses to Measles virus N protein, a negative control, are also shown.

### DETAILED DESCRIPTION

Before the subject matter is herein described, it is to be understood that this disclosure is not limited to particular viral particles, compositions, methods or experimental conditions described, as such viral particles, compositions, methods and conditions may vary. It is also to be understood that the terminology used herein is for the purpose of describing particular embodiments only.

### Definitions

Unless defined otherwise, all technical and scientific terms used herein have the same meaning as commonly understood by one of ordinary skill in the art to which this disclosure belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “comprise(s),” “include(s),” “having,” “has,” and “contain(s),” are intended to be open-ended transitional phrases, terms, or words that do not preclude the possibility of additional acts or structures.

“Antibody” as used herein encompasses polyclonal and monoclonal antibodies and refers to immunoglobulin molecules of classes IgA (e.g., IgA1 or IgA2), IgD, IgE, IgG (e.g., IgG1, IgG2, IgG3 and IgG4) or IgM, or fragments, or derivatives thereof, including without limitation Fab, F(ab′)2, Fd, single chain antibodies, diabodies, bispecific antibodies, bifunctional antibodies, humanized antibodies, and various derivatives thereof.

In the context of the present disclosure, the term “neutralizing antibody” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

As used herein in connection with a viral infection and vaccination, the terms “protective immune response” or “protective immunity” refer to an immune response that that confers some benefit to the subject in that it prevents or reduces the infection or prevents or reduces the development of a disease associated with the infection. Without wishing to be bound by theory, the presence of SARS-CoV-2 neutralizing antibodies in a subject can indicate the presence of a protective immune response in the subject.

The terms “immunogenic composition”, “vaccine composition”, or “vaccine”, which are used interchangeably, refer to a composition comprising at least one immunogenic and/or antigenic component that induces an immune response in a subject (e.g., humoral and/or cellular response). In certain embodiments, the immune response is a protective immune response. A vaccine may be administered for the prevention or treatment of a disease, such as an infectious disease. A vaccine composition may include, for example, live or killed infectious agents, recombinant infectious agents (e.g., recombinant viral particles, virus-like particles, nanoparticles, liposomes, or cells expressing immunogenic and/or antigenic components), antigenic proteins or peptides, nucleic acids, etc. Vaccines may be administered with an adjuvant to boost the immune response.

The term “operably linked” includes a linkage of nucleic acid elements in a functional relationship. A nucleic acid sequence is “operably linked” when it is placed into a functional relationship with another nucleic acid sequence. For instance, a promoter or enhancer, or a 5′ regulatory region containing a promoter or enhancer, is operably linked to a coding sequence if it effects the transcription of the coding sequence.

The terms “derivative” and “variant” are used herein interchangeably to refer to an entity that has significant structural identity with a reference entity but differs structurally from the reference entity in the presence or level of one or more chemical moieties as compared with the reference entity. In many embodiments, a derivative also differs functionally from its reference entity. In general, whether a particular entity is properly considered to be a “derivative” of a reference entity is based on its degree of structural identity with the reference entity. As will be appreciated by those skilled in the art, any biological or chemical reference entity has certain characteristic structural elements. A derivative, by definition, is a distinct entity that shares one or more such characteristic structural elements. To give but a few examples, a small molecule may have a characteristic core structural element (e.g., a macrocycle core) and/or one or more characteristic pendent moieties so that a derivative of the small molecule is one that shares the core structural element and the characteristic pendent moieties but differs in other pendent moieties and/or in types of bonds present (single vs double, E vs Z, etc.) within the core. A derivative nucleic acid may have a characteristic sequence element comprised of a plurality of nucleotide residues having designated positions relative to one another in linear or three-dimensional space. In some embodiments, the nucleic acid sequence of a derivative may be 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identical over the full length of the reference sequence or a fragment thereof. A derivative peptide or polypeptide may have a characteristic sequence element comprised of a plurality of amino acids having designated positions relative to one another in linear or three-dimensional space and/or contributing to a particular biological function. Derivative peptides and polypeptides include peptides and polypeptides that differ in amino acid sequence from the reference peptide or polypeptide by the insertion, deletion, and/or substitution of one or more amino acids, but retain at least one biological activity of such reference peptide or polypeptide (e.g., the ability to mediate cell infection by a virus, the ability to mediate membrane fusion, the ability to be bound by a specific antibody or to promote an immune response, etc.). In some non-limiting embodiments, a derivative peptide or polypeptide shows the sequence identity over the full length with the reference peptide or polypeptide (or a fragment thereof) that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more. Alternatively or in addition, a derivative peptide or polypeptide may differ from a reference peptide or polypeptide as a result of one or more and/or one or more differences in chemical moieties attached to the polypeptide backbone (e.g., in glycosylation, phosphorylation, acetylation, myristoylation, palmitoylation, oxidation, formylation, amidation, polyglutamylation, ADP-ribosylation, pegylation, biotinylation, etc.). In some embodiments, a derivative peptide or polypeptide lacks one or more of the biological activities of the reference polypeptide or has a reduced or increased level of one or more biological activities as compared with the reference polypeptide. Derivatives of a particular peptide or polypeptide may be found in nature or may be synthetically or recombinantly produced. As used herein, the term “derivative” or “variant” also encompassed various fusion proteins and conjugates, including fusions or conjugates with detection tags (e.g., HA tag, histidine tag, biotin, fusions with fluorescent or luminescent domains, etc.), dimerization/multimerization sequences, Fc, signaling sequences, etc.

The term “coronavirus” as used herein refers to the subfamily Coronavirinae within the family Coronaviridae, within the order Nidovirales. Based on the phylogenetic relationships and genomic structures, this subfamily consists of four genera: Alphacoronavirus, Betacoronavirus, Gammacoronavirus and Deltacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-CoV-2, SARS-CoV and MERS-CoV, which cause severe respiratory syndrome in humans. The other four human coronaviruses, HCoV-NL63, HCoV-229E, HCoV-OC43 and HKU1, induce only mild upper respiratory diseases in immunocompetent hosts, although some of them can cause severe infections in infants, young children and elderly individuals. Additional non-limiting examples of commercially important coronaviruses include transmissible gastroenteritis coronavirus (TGEV), porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus. Reviewed in Cui et al., Nature Reviews Microbiology, 2019, 17:181-192; Fung et al., Annu. Rev. Microbiol., 2019, 73:529-557.

The term “rhabdovirus” as used herein refers to Rhabdoviridae family of viruses in the order Mononegavirales encompassing more than 150 viruses of vertebrates, invertebrates and plants. Examples of rhabdoviruses include rabies virus (RABV) from the Lyssavirus genus, vesiculoviruses from Vesiculovirus genus, the viral hemorrhagic septicemia virus (VHSV) and infectious hematopoietic necrosis virus, both from the Novirhabdovirus genus. Rhabdoviruses are bullet-shaped enveloped viruses with negative-sense single-stranded RNA genome 11-15 kb in length. The genome of rhabdoviruses comprises up to ten genes among which only five are common to all members of the family. These genes encode the nucleoprotein (N), the phosphoprotein (P), the matrix protein (M), the glycoprotein (G) and the viral polymerase (also known as large protein) (L). The genome is associated with N, L and P to form the nucleocapsid, which is condensed by the M protein into a tightly coiled helical structure. The condensed nucleocapsid is surrounded by a lipid bilayer containing the viral glycoprotein G that constitutes the spikes that protrude from the viral surface. Rhabdoviruses enter the cell via the endocytic pathway and subsequently fuse with the cellular membrane within the acidic environment of the endosome. Both receptor recognition and membrane fusion are mediated by a single transmembrane viral glycoprotein (G). Fusion between the viral envelope and the endosomal membrane is triggered via a low-pH induced (in the endosome) structural rearrangement of the G resulting in the release the viral genome and associated proteins into the cytoplasm of target cells.

As used herein, the term “vesiculovirus” refers to any virus in the Vesiculovirus genus. Non-limiting examples of vesiculoviruses include, e.g., Vesicular Stomatitis Virus (VSV) (e.g., VSV-New Jersey, VSV-Indiana), Alagoas vesiculovirus, Cocal vesiculovirus, Jurona vesiculovirus, Carajas vesiculovirus, Maraba vesiculovirus, Piry vesiculovirus, Calchaqui vesiculovirus, Yug Bogdanovac vesiculovirus, Isfahan vesiculovirus, Chandipura vesiculovirus, Perinct vesiculovirus, Porton-S vesiculovirus. Vesicular Stomatitis Virus (VSV), in the Vesiculovirus genus, is a prototypic rhabdovirus. While VSV is used as an example in the present disclosure, this disclosure can also be used for other vesiculoviruses and other rhabdoviruses. There are two major serotypes of VSV, New Jersey and Indiana, both of which can infect insects and mammals, causing economically important diseases in cattle, equines and swine. The VSV genome is composed of single-stranded, negative-sense RNA of 11-12 kb, which encodes five viral proteins: the nucleoprotein (N), the phosphoprotein (P), the matrix protein (M), the glycoprotein (G) and the viral polymerase (also known as large protein) (L). G monomers associate to form trimeric spikes anchored in the viral membrane. Reviewed in, e.g., Sun et al., Future Virol., 2010, 5(1):85-96 and Aurélie et al., Viruses 2012, 4:117-139.

As used herein, the phrase “non-essential portion(s) of the recombinant VSV genome” or variations thereof refers to a region of the VSV genome that can be modified without affecting the development and/or growth of the virus in vitro and/or in vivo and without affecting the virus's functions required to act as an immunogenic and/or antigenic composition or vaccine.

As used herein, the term “foreign” refers to a heterologous gene, protein, or peptide that is not naturally part of the VSV genome or naturally expressed in the wild-type VSV. The foreign protein or peptide is one that can function as an antigen for the induction of an immune response.

As used herein in connection with various recombinant enveloped viral particles, the term “pseudotyped” refers to viral particles comprising in their lipid envelope molecules, e.g., proteins, glycoproteins, etc, which are mutated and/or heterologous compared to molecules typically found on the surface of a virus from which the particles are derived (i.e., a “reference virus”), and which may affect, contribute to, direct, redirect and/or completely change the tropism of the viral particle in comparison to the reference virus. In some embodiments, a viral particle is pseudotyped such that it recognizes, binds and/or infects a target (ligand or cell) that is different to that of the reference virus. In some embodiments, a viral particle is pseudotyped such that it does not recognize, bind, and/or infect a target (ligand or cell) of the reference virus.

The term “fusogen” or “fusogenic molecule” is used herein to refer to any molecule that can trigger membrane fusion when present on the surface of a virus particle. A fusogen can be, for example, a protein (e.g., a viral glycoprotein) or a fragment or derivative thereof.

The term “replication-competent” is used herein to refer to viruses (including wild-type and recombinant viral particles) that are capable of infecting and propagating within a susceptible cell.

The term “encoding” can refer to encoding from either the (+) or (−) sense strand of the polynucleotide for expression in the virus particle.

The term “effective” applied to dose or amount refers to that quantity of a compound (e.g., a recombinant virus) or composition (e.g., pharmaceutical, vaccine or immunogenic and/or antigenic composition) that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

As used herein, the phrase “a subject in need thereof” means a human or non-human animal that exhibits one or more symptoms or indicia of a disease or disorder associated with a coronavirus infection, and/or who is at risk of developing a disease or disorder associated with an infection. In certain embodiments, the coronavirus is SARS-CoV-2. In certain embodiments, the disease or disorder is COVID-19. In certain embodiments, the COVID-19 disease symptoms include, but are not limited to, fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock and death in severe cases.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. Non-limiting examples of the symptoms of the COVID-19 disease, include, without limitation, fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock, and death. When used in connection with a disease caused by a viral infection (e.g., SARS-CoV-2 infection), the terms “prevent”, “preventing” or “prevention” refer to prevention of spread of infection in a subject exposed to the virus, e.g., prevention of the virus from entering the subject's cells.

The terms “individual” or “subject” or “patient” or “animal” refers to humans, veterinary animals (e.g., cats, dogs, cows, horses, sheep, pigs, etc.) and experimental animal models of diseases (e.g., mice, rats, ferrets, monkeys, etc.). In a preferred embodiment, the subject is a human.

The terms “nucleic acid”, “polynucleotide” and “nucleotide” are used interchangeably and encompass both DNA and RNA, including positive- and negative-stranded, single- and double-stranded, unless specified otherwise.

The phrase “pharmaceutically acceptable”, as used in connection with compositions described herein, refers to molecular entities and other ingredients of such compositions that are physiologically tolerable and do not typically produce untoward reactions when administered to a subject (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The practice of the present disclosure employs, unless otherwise indicated, conventional techniques of statistical analysis, molecular biology (including recombinant techniques), virology, microbiology, cell biology, chemistry and biochemistry, which are within the skill of the art. Such tools and techniques are described in detail in e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### General Description

Coronaviruses form enveloped and spherical particles of 80-160 nm in diameter. They contain a positive-sense, non-segmented, single-stranded RNA (ssRNA) genome of 27-32 kb in size. The 5′-terminal two-thirds of the genome encodes polyproteins, pp1a and pp1ab. The 3′ terminus encodes structural proteins, including envelope glycoproteins spike (S), envelope (E), membrane (M) and nucleocapsid (N). The genomic RNA is 5′-capped and 3′-polyadenylated and contains multiple open reading frames (ORFs). The invariant gene order is 5′-replicase-S-E-M-N-3′, with numerous small ORFs (encoding accessory proteins) scattered among the structural genes. The coronavirus replicase is encoded by two large overlapping ORFs (ORF1a and ORF1b) occupying about two-thirds of the genome and is directly translated from the genomic RNA (gRNA). The structural and accessory genes, however, are translated from subgenomic RNAs (sgRNAs) generated during genome transcription/replication. Infection starts with the attachment of the coronavirus to the cognate cellular receptor, which induces endocytosis. Membrane fusion typically occurs in the endosomes, releasing the viral nucleocapsid to the cytoplasm. The genomic RNA (gRNA) serves as the template for translation of polyproteins pp1a and pp1ab, which are cleaved to form nonstructural proteins (nsps). NSPs induce the rearrangement of cellular membrane to form double-membrane vesicles (DMVs), where the viral replication transcription complexes (RTCs) are anchored. Full-length gRNA is replicated via a negative-sense intermediate, and a nested set of subgenomic RNA (sgRNA) species are synthesized by discontinuous transcription. These sgRNAs encode viral structural and accessory proteins. Particle assembly occurs in the ER-Golgi intermediate complex (ERGIC), and mature virions are released in smooth-walled vesicles via the secretory pathway.

Coronavirus entry into host cells is mediated by the transmembrane spike (S) glycoprotein (also referred to as “spike glycoprotein”, “S glycoprotein”, “S protein” or “spike protein”). S glycoprotein forms homotrimers protruding from the viral surface. S glycoprotein comprises two functional subunits responsible for binding to the host cell receptor (S1 subunit) and fusion of the viral and cellular membranes (S2 subunit). For many coronaviruses, including SARS-CoV and SARS-CoV-2, S glycoprotein is cleaved at the boundary between the S1 and S2 subunits, which remain non-covalently bound in the prefusion conformation. The distal S1 subunit comprises the receptor-binding domain(s) (RBD) and contributes to stabilization of the prefusion state of the membrane-anchored S2 subunit that contains the fusion machinery. The S glycoprotein is further cleaved by host proteases at the ST site located immediately upstream of the fusion peptide. This cleavage has been proposed to activate the protein for membrane fusion via conformational changes. Walls et al., Cell, published online Mar. 9, 2020; available at doi.org/10.1016/j.cell.2020.02.058.

SARS-CoV and SARS-CoV-2 interact directly with angiotensin-converting enzyme 2 (ACE2) to enter target cells (Hoffmann et al., Cell, 2020, 181:1-10; available at doi.org/10.1016/j.cell.2020.02.052). SARS-S and SARS-CoV-2-S share 76% amino acid identity. Six receptor binding domain (RBD) amino acids have been shown to be critical for binding to ACE2 receptors and for determining the host range of SARS-CoV-like viruses. They are Y442, L472, N479, D480, T487 and Y4911 in SARS-CoV, which correspond to L455, F486, Q493, S494, N501 and Y505 in SARS-CoV-2 (Andersen et al., Nature Medicine, 2020).

Currently, there are no useful treatments or vaccines available to treat and/or prevent a SARS-CoV-2 infection. The present disclosure, while applicable to various epitopes on SARS-CoV-2, focuses its therapeutic and vaccine design on the S glycoprotein found on the surface of SARS-CoV-2 as the main target of anti-viral neutralizing antibodies, due to the role of this glycoprotein in viral attachment and fusion with the host cell. Thus, the immunogenic and/or antigenic compositions and vaccine produce antibodies to the SARS-CoV-2 S glycoprotein that may directly neutralize the coronavirus, or block fusion of the virus with the cell.

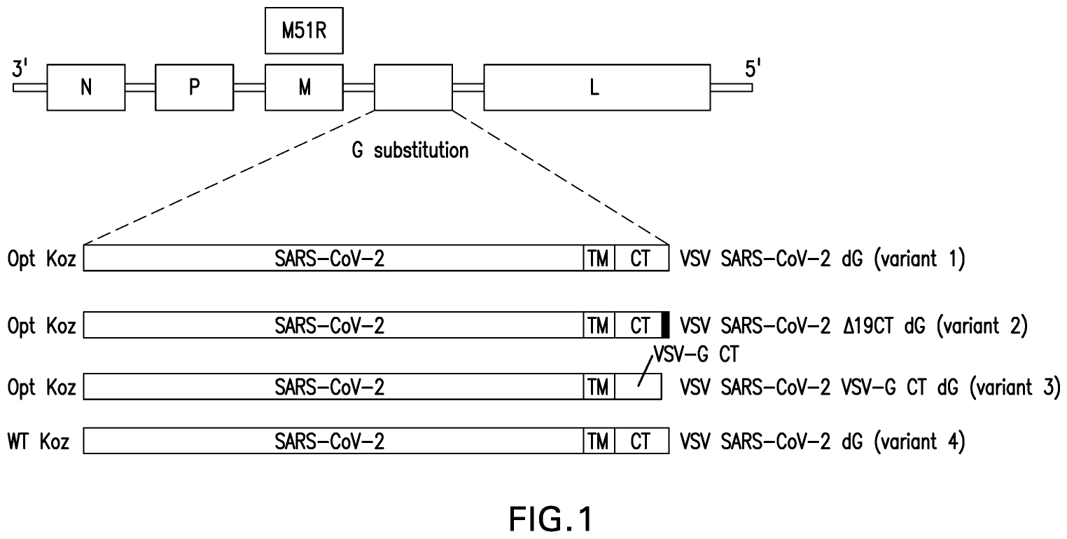

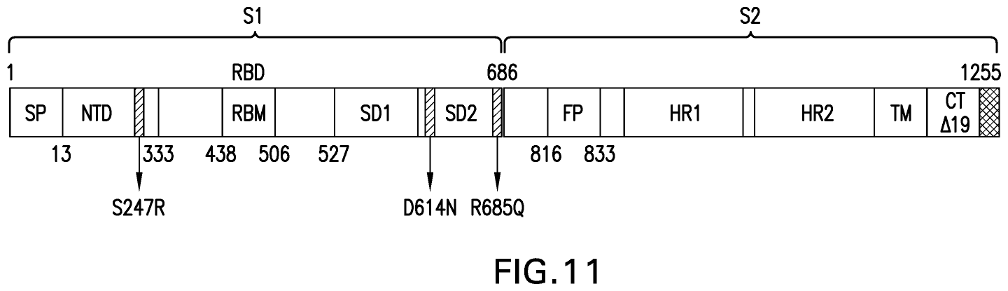

In certain aspects, the disclosure provides for recombinant vesicular stomatitis virus (VSV) particles, wherein the VSV genome encodes at least one SARS-CoV-2 S glycoprotein (NCBI Reference Sequence: NC_045512.2; Protein_ID: YP_009724390.1; SEQ ID NO: 1) or fragment or derivative thereof (e.g. SEQ ID NO: 3, SEQ ID NO: 5, SEQ ID NO: 20, and SEQ ID NO: 22). See FIGS. 1 and 11. The fragment may be derived from any of the known regions of SARS-CoV-2 S glycoprotein, such as 51, S2, or the RBD (see Walls et al., Cell, published online Mar. 9, 2020; available at doi.org/10.1016/j.cell.2020.02.058).

In certain aspects, the recombinant VSV particles disclosed herein can be used in immunogenic and/or antigenic compositions or vaccines. In certain embodiments, the immunogenic and/or antigenic compositions or vaccines can be used in the treatment or prevention of a disease or disorder in a subject infected with SARS-CoV-2. In certain embodiments, the disease or disorder is COVID-19.

In certain aspects, the recombinant VSV particles disclosed herein can be used to treat or prevent a disease or disorder in a subject infected with SARS-CoV-2 comprising administering to a subject in need of such treatment or prevention one or more of the recombinant VSV particles.

In certain aspects, the recombinant VSV particles disclosed herein can be used to diagnose and/or monitoring progression of a SARS-CoV-2 infection or COVID-19 disease, including response to vaccination and/or therapy.

In certain embodiments, the recombinant VSV particles disclosed herein can be used as a live vaccine, or can be inactivated for use as a killed vaccine.

In certain embodiments, the recombinant VSV particles disclosed herein can also be used to produce large quantities of readily purified antigen, e.g., for use in subunit vaccines or to generate neutralizing anti-SARS-CoV2 antibodies.

### Recombinant Viral Particles

One aspect of the disclosure provides recombinant rhabdoviral particles. The Rhabdoviridae family is mainly composed of a cage, bullet-shaped or bacilliform virus and has a negative-sense single-stranded RNA genome that infects vertebrates, invertebrates or plants. Several Rhabdoviridae members are being developed as live-attenuated vaccine vectors for the prevention or treatment of infectious disease and cancer. Non-limiting examples of rhabdoviruses useful in this disclosure is rabes, cytolabudoviruses, dicholabdoviruses, ephemeraviruses, lyssaviruses, nobilabdoviruses and vesiculoviruses.

One aspect of the disclosure provides recombinant vesiculoviruses particles. Many vesiculoviruses are known in the art and can be made recombinant according to the methods disclosed herein. Examples of such vesiculoviruses are listed in table 1.

TABLE 1
Examples of Vesiculoviruses

| Virus | Source of virus in nature |
|---|---|
| VSV-New Jersey | Mammals, mosquitoes, midges, blackflies, houseflies |
| VSV-Indiana | Mammals, mosquitoes, sandflies |
| Alagoas | Mammals, sandflies |
| Cocal | Mammals, mosquitoes, mites |
| Jurona | Mosquitoes |
| Carajas | Sandflies |
| Maraba | Sandflies |
| Piry | Mammals |
| Calchaqui | Mosquitoes |
| Yug Bogdanovac | Sandflies |
| Isfahan | Sandflies, ticks |
| Chandipura | Mammals, sandflies |
| Perinct | Mosquitoes, sandflies |
| Porton-S | Mosquitoes |

One aspect of the disclosure provides recombinant vesicular stomatitis virus (VSV) particles. VSV is an attractive virus for production of recombinant viral particles, because it can be produced in high titers and does not cause serious pathology in humans. In certain embodiments, in the recombinant VSV particles as described herein, the VSV glycoprotein (G protein) is replaced by a coronavirus spike (S) glycoprotein or a fragment or a derivative thereof. In certain embodiments, the recombinant VSV is a recombinant VSV-New Jersey or VSV-Indiana. In certain embodiments, the recombinant VSV is a recombinant VSV-Indiana. While VSV is used as an example in the present disclosure, this disclosure can also be used for other vesiculoviruses and other rhabdoviruses.

VSV comprises a single (non-segmented) negative-stranded genomic RNA that is generally transcribed by a virion polymerase into five mRNAs encoding five structural proteins. The five structural proteins include G protein, large protein (L), phosphoprotein (P), matrix protein (M) and nucleoprotein (N). The nucleocapsid protein encapsulates the RNA genome. Two proteins that form a polymerase complex are bound to the nucleocapsid. The M protein is associated with the nucleocapsid and the membrane. A single (transmembrane) envelope G protein extends from the viral envelope. The VSV G protein functions to bind virus to a cellular receptor and to catalyze fusion of the viral membrane with cellular membranes to initiate the infectious cycle. The size of the VSV genome is about 11 kilobases.

VSV can be transmitted to a variety of mammalian hosts, generally cattle, horses, swine and rodents. VSV infection of humans is uncommon, and in general is either asymptomatic or characterized by mild flu-like symptoms that resolve in three to eight days without complications. VSV is not considered a human pathogen and pre-existing immunity to VSV is rare in the human population making VSV an attractive viral vector for vaccine and therapeutic applications. Other beneficial characteristics of VSV include, but are not limited to, (i) ability to replicate robustly in cell culture, (ii) inability to either integrate into host cell DNA or undergo genetic recombination, (iii) multiple serotypes can allow for prime-boost immunization strategies, and (iv) foreign genes of interest can be inserted into the VSV genome and expressed abundantly by the viral transcriptase.

Fusion of rhabdoviruses (e.g., VSV) to cells, and their subsequent uptake, is described in Belot, L. et al., “Structural and cellular biology of rhabdovirus entry”, Adv. Virus Res., 2019, 104:147-183, which is incorporated by reference herein in its entirety, and Albertini, A. A. V. et al., “Molecular and Cellular Aspects of Rhabdovirus Entry” Viruses, 2012, 4:117-139, which is incorporated by reference herein in its entirety. Further description of endocytosis of VSV is found in Sun, X. et al., “Internalization and fusion mechanism of vesicular stomatitis virus and related rhabdoviruses” Future Virol., 2010, 5(1):85-96, which is incorporated by reference herein in its entirety. For general information on virus-cell fusion, see Igonet, S. et al., “SnapShot: Viral and Eukaryotic Protein Fusogens” Cell, 2012, 151:1634e1, which is incorporated by reference herein in its entirety.

Cell-cell fusion mediated by other viruses, such as HIV virus, has been described in Kondo, N. et al., “Conformational changes of the HIV-1 envelope protein during membrane fusion are inhibited by the replacement of its membrane-spanning domain” J. Biol. Chem., 2010, 285(19):14681-88, which is incorporated by reference herein in its entirety.

In certain embodiments, the recombinant VSV particle is a replication-competent viral particle. In certain embodiments, the recombinant VSV particle is a replication-defective viral particle.

In certain aspects, the recombinant VSV particles can be used in immunogenic and/or antigenic compositions or vaccines. In certain embodiments, the immunogenic and/or antigenic compositions and vaccines described herein use only one type of recombinant VSV particles. In certain embodiments, the immunogenic and/or antigenic compositions and vaccines described herein use more than one type of recombinant VSV particles. In certain embodiments, such immunogenic and/or antigenic compositions and vaccines use a mixture of two or more recombinant VSV particles encoding different coronaviral S glycoproteins (e.g., SARS-CoV-2 S glycoproteins originating from different viral strains, variants or mutants). In certain embodiments, immunogenic and/or antigenic compositions and vaccines can be used in the treatment or prevention of a disease or disorder in a subject infected with SARS-CoV-2. In certain embodiments, the disease or disorder is COVID-19.

In certain aspects, the recombinant VSV particles can be used to diagnose and/or monitoring progression of a disease or disorder in a subject infected with SARS-CoV-2, including response to vaccination and/or therapy. In certain embodiments, the disease or disorder is COVID-19.

In certain aspects, the current disclosure provides cells for the production of the recombinant VSV particles described herein. Exemplary cells include, but are not limited to, any cell in which VSV grows, e.g., mammalian cells and some insect (e.g., Drosophila) cells. A vast number of primary cells and cell lines commonly known in the art can be used as host or packaging cells. By way of example, useful cell lines include but are not limited to BHK (baby hamster kidney) cells, CHO (Chinese hamster ovary) cells, HeLA (human) cells, mouse L cells, Vero (monkey) cells, ESK-4, PK-15, EMSK cells, MDCK (Madin-Darby canine kidney) cells, MDBK (Madin-Darby bovine kidney) cells, 293 (human) cells, Hep-2 cells, primary chick embryo fibroblasts, primary chick embryo fibroblasts, quasi-primary continuous cell lines (e.g. AGMK-African green monkey kidney cells), human diploid primary cell lines (e.g. WI-38 and MRCS cells), and Monkey Diploid Cell Line (e.g. FRhL-Fetal Rhesus Lung cells).

### VSV DNA for Transcription to Produce VSV Antigenomic (+) RNA

Recombinant VSV particles described herein can be produced using methods known in the art, e.g., by providing in an appropriate host cell: (a) DNA that can be transcribed to encode VSV antigenomic (+) RNA (complementary to the VSV genome), (b) a recombinant source of VSV nucleoprotein (N) protein, (c) a recombinant source of VSV phosphoprotein (P) protein, (d) a recombinant source of VSV large protein (L), and (e) foreign DNA; under conditions such that the DNA is transcribed to produce the antigenomic RNA, and a VSV is produced that contains genomic RNA complementary to the antigenomic RNA produced and foreign RNA, which is not naturally a part of the VSV genome, from the DNA. Methods and compositions useful for generating recombinant VSV particles may be found, for example, in U.S. Pat. Nos. 7,153,510; 9,861,668; 8,012,489; 9,630,996; 8,287,878; 9,248,178 U.S. Patent Publication Nos. 2014/0271564; 2012/0121650; Fukishi et al., J. Gen. Virol., 2005, 86:2269-2274, each of which are incorporated by reference herein in their entirety.

In certain embodiments, the foreign RNA contained within the genome of the recombinant VSV, upon expression in an appropriate host cell, produces one or more foreign protein or peptide. In certain embodiments, the one or more foreign protein or peptide is immunogenic and/or antigenic. In certain embodiments, one foreign protein is a coronavirus spike (S) glycoprotein (e.g., S glycoprotein from SARS-CoV-2) or a fragment or derivative thereof as described in greater detail below.

In certain alternative embodiments, the one or more foreign proteins (e.g., a coronavirus S glycoprotein) are not encoded by the genome of the recombinant VSV particle but are incorporated into said VSV particle as proteins upon production of the recombinant viral particles. In certain embodiments, the recombinant VSV particle may encode the coronaviral S glycoprotein in the VSV viral genome. Alternatively, the VSV particle may be pseudotyped with the coronaviral S glycoprotein without it being encoded in the genome (e.g., by using a separate plasmid in a packaging cell).

In certain embodiments, in addition to encoding a coronavirus spike (S) glycoprotein (e.g., S glycoprotein from SARS-CoV-2) or a fragment or derivative thereof, the genome of the recombinant VSV encodes a reporter protein. Non-limiting examples of reporter proteins include, e.g., luciferases (including but not limited to, Renilla luciferase or a mutant thereof, (dCpG)Luciferase, NanoLuc reporter, firefly luciferase, MetLuc, Vibrio fischeri lumazine protein, Vibrio harveyi luminaze protein, inoflagellate luciferase, firefly luciferase YY5 mutant, firefly luciferase LGR mutant, firefly luciferase mutant E, and derivatives thereof) and fluorescent proteins (including but not limited to, green fluorescent protein (GFP) [e.g., Aequorea victoria GFP, Renilla muelleri GFP, Renilla reniformis GFP, Renilla ptilosarcus GFP], GFP-like fluorescent proteins, (GFP-like), enhanced green fluorescent protein (EGFP), yellow fluorescent protein (YFP) [e.g., Topaz, Venus, mCitrine, YPet, TagYFP, PhiYFP, ZsYellow1, mBanana], enhanced yellow fluorescent protein (EYFP), blue fluorescent protein (BFP) [e.g., EBFP2, Azurite, GFP2, GFP10, and mTagBFP], enhanced blue fluorescent protein (EBFP), cyan fluorescent protein (CFP) [e.g., mECFP, Cerulean, CyPet, AmCyan1, Midori-Ishi Cyan, TagCFP, mCFPmm, mTFP1 (Teal)], enhanced cyan fluorescent protein (ECFP), superfolder GFP, superfolder YFP, orange fluorescent protein [e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine], red fluorescent protein [e.g., mRuby, mApple, mStrawberry, AsRed2, mRFP1, JRed, mCherry, HcRedl, mRaspberry, dKeima-Tandem, HcRed-Tandem, mPlum, tdTomato, AQ143], small ultrared fluorescent protein, FMN-binding fluorescent protein, dsRed, qFP611, Dronpa, TagRFP, KFP, EosFP, IrisFP, Dendra, Kaede, KikGr1, emerald fluorescent protein, Azami Green, mWasabi, TagGFP, TurboGFP, AcGFP, ZsGreen, T-Sapphire, and derivatives thereof), β-galactosidase, β-glucuronidase, β-geo, etc.

Any DNA that can be transcribed to produce VSV antigenomic (+) RNA (complementary to the VSV genome) can be used for the construction of a recombinant DNA containing foreign DNA encoding a heterologous (foreign) protein or peptide, for use in producing the recombinant VSV particles described herein. In certain embodiments, the DNA that can be transcribed to encode VSV antigenomic (+) RNA comprises at least genes for the VSV N protein, the VSV P protein, and the VSV L protein. In certain embodiments, the DNA that can be transcribed to encode VSV antigenomic (+) RNA comprises at least genes for the VSV N protein, the VSV P protein, the VSV L protein, and the foreign protein or peptide. In certain embodiments, DNA that can be transcribed to encode VSV antigenomic (+) RNA can further encode the VSV matrix (M) protein and/or G glycoprotein.

The VSV vector can be genetically modified to include one or more mutations or “mutation classes” in the genome. “Mutation class”, “mutation classes” or “classes of mutation” are used interchangeably, and refer to mutations known in the art, when used singly, to attenuate VSV. Exemplary mutation classes include, but are not limited to, a VSV temperature-sensitive N gene mutation (hereinafter, “N(ts)”), a temperature-sensitive L gene mutation (hereinafter, “L(ts)”), a point mutation, a G-stem mutation (hereinafter, “G(stem)”), a non-cytopathic M gene mutation (hereinafter, “M(ncp)”), a gene shuffling or rearrangement mutation, a truncated G gene mutation (hereinafter, “G(ct)”), an ambisense RNA mutation, a G gene insertion mutation, a gene deletion mutation and the like. Mutations can be insertions, deletions, substitutions, gene rearrangement or shuffling modifications.

The mutations can attenuate the infectivity, virulence or pathogenic effects of VSV. The attenuation can be additive or synergistic. With synergistic attenuation, the level of VSV attenuation is greater than additive. Synergistic attenuation of VSV can arise from combining at least two classes of mutation in the same VSV genome, thereby resulting in a reduction of VSV pathogenicity much greater than an additive attenuation level observed for each VSV mutation class alone. A synergistic attenuation of VSV can provide for an LD₅₀ at least greater than the additive attenuation level observed for each mutation class alone (i.e., the sum of the two mutation classes), where attenuation levels (i.e., the LD₅₀) are determined in a small animal neurovirulence model.

The VSV M gene encodes the virus matrix (M) protein, and two smaller in-frame polypeptides (M2 and M3). The M2 and M3 polypeptides can be translated from the same open reading frame (ORF) as the M protein and lack the first 33 and 51 amino acids, respectively. A recombinant VSV vector comprising non-cytopathic M gene mutations (i.e., VSV vectors that also do not express M2 and M3 proteins) can be generated, and can further comprise one or more additional mutation(s) thereby resulting in a VSV vector that was highly attenuated in cell culture and in animals.

In certain embodiments, the recombinant VSV particles described herein comprise a non-cytopathic mutation in the M gene. The VSV (Indiana serotype) M gene encodes a 229 amino acid M (matrix) protein in which the first thirty amino acids of the NH2-terminus comprise a proline-rich PPPY (PY) motif. The PY motif of VSV M protein is located at amino acid positions 24-27 in both VSV Indiana (Genbank Accession Number X04452) and New Jersey (Genbank Accession Number M14553) serotypes. The VSV may comprise mutations in the PY motif (e.g., APPY, AAPY, PPAY, APPA, AAPA and PPPA). The VSV can comprise any of various amino acid mutations (e.g., deletions, substitutions, insertions, etc.) into the M protein PSAP (PS) motif. These and other mutations in the PY motif may be effective to reduce virus yield by blocking a late stage in virus budding.

In certain embodiments, the DNA that can be transcribed to encode VSV antigenomic (+) RNA comprises a gene that encodes a VSV M protein. In certain embodiments, the VSV M protein used in the methods, compositions, or vaccines described herein may comprise or consist of the amino acid sequence of SEQ ID NO: 9, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 9. In certain embodiments, the polynucleotide sequence encoding the VSV M protein may comprise or consist of the polynucleotide sequence of SEQ ID NO: 10, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the polynucleotide sequence of SEQ ID NO: 10.

The recombinant VSV particles described herein may comprise one or more M gene mutations. Non-limiting examples of M protein mutations include, e.g., a glycine changed to a glutamic acid at position (21), a leucine changed to a phenylalanine at position (111), a methionine changed to an arginine at position (51), a glycine changed to a glutamic acid at position (22), a methionine changed to an arginine at position (48), a leucine changed to a phenylalanine at position (110), a methionine changed to an alanine at position (51), and a methionine changed to an alanine at position (33). See, e.g., U.S. Pat. No. 9,630,996. In various embodiments of the methods described herein, the genome of the recombinant VSV encodes a mutant VSV matrix M protein comprising the M51R variant M protein. Variant M51R eliminates M protein's ability to block cellular nucleo-cytoplasmic transport and thus substantially attenuates VSV infectivity.

In certain embodiments, the DNA that can be transcribed to encode VSV antigenomic (+) RNA comprises a gene that encodes a mutant VSV M protein. In certain embodiments, the DNA that can be transcribed to encode VSV antigenomic (+) RNA comprises a gene that encodes a VSV M protein comprising a mutation at methionine (M) 51. In certain embodiments, the mutation is from methionine (M) to arginine (R). In certain embodiments, the DNA that can be transcribed to encode VSV antigenomic (+) RNA comprises a gene that encodes a VSV M protein comprising a deletion at methionine (M) 51. In certain embodiments, the mutated VSV M protein used in the vaccines or methods, compositions, or vaccines described herein may comprise or consist of the amino acid sequence of SEQ ID NO: 7, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 7. In certain embodiments, the polynucleotide sequence encoding the VSV M protein may comprise or consist of the polynucleotide sequence of SEQ ID NO: 8, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the polynucleotide sequence of SEQ ID NO: 8.

DNA that can be transcribed to produce VSV (for example) antigenomic (+) RNA (such DNA being referred to herein as “VSV (−) DNA”) is available in the art and/or can be obtained by standard methods. VSV (−) DNA for any serotype or strain known in the art, e.g., the New Jersey or Indiana serotypes of VSV, can be used. The complete nucleotide and deduced protein sequence of the VSV genome is known, and is available as Genbank VSVCG, Accession No. J02428; NCBI Seq ID 335873; and is published in Rose and Schubert, 1987, in The Viruses: The Rhabdoviruses, Plenum Press, NY, pp. 129-166. An example of the complete sequence of the VSV(−) DNA that is contained in plasmid pVSVFL(+) is shown in U.S. Pat. No. 7,153,510, which is incorporated herein in its entirety for all intended purposes. Sequences of other vesiculovirus genomes have been published and are available in the art.

VSV (−) DNA, if not already available, can be prepared by standard methods, as follows: VSV genomic RNA can be purified from virus preparations, and reverse transcription with long distance polymerase chain reaction used to generate the v (−) DNA. Alternatively, after purification of genomic RNA, VSV mRNA can be synthesized in vitro, and cDNA prepared by standard methods, followed by insertion into cloning vectors (see, e.g., Rose and Gallione, 1981, J. Virol. 39(2):519-528). Individual cDNA clones of VSV RNA can be joined by use of small DNA fragments covering the gene junctions, generated by use of reverse transcription and polymerase chain reaction (RT-PCR) (Mullis and Faloona, 1987, Meth. Enzymol. 155:335-350) from VSV genomic RNA (see Section 6, infra). VSV and other vesiculoviruses are available in the art.

In certain embodiments, one or more, usually unique, restriction sites (e.g., in a polylinker) are introduced into the VSV (−) DNA, in intergenic regions, or 5′ of the sequence complementary to the 3′ end of the VSV genome, or 3′ of the sequence complementary to the 5′ end of the VSV genome, to facilitate insertion of the foreign DNA.

In certain embodiments, the VSV (−) DNA is constructed so as to have a promoter operatively linked thereto. The promoter should be capable of initiating transcription of the (—) DNA in an animal or insect cell in which it is desired to produce the recombinant VSV. Promoters which may be used include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); heat shock promoters (e.g., hsp70 for use in Drosophila S2 cells); the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); and myosin light chain-2 gene control region which is active in skeletal muscle (Sani, 1985, Nature 314:283-286). Preferably, the promoter is an RNA polymerase promoter, preferably a bacteriophage or viral or insect RNA polymerase promoter, including but not limited to the promoters for T7 RNA polymerase, SP6 RNA polymerase, and T3 RNA polymerase. If an RNA polymerase promoter is used in which the RNA polymerase is not endogenously produced by the host cell in which it is desired to produce the recombinant VSV, a recombinant source of the RNA polymerase must also be provided in the host cell.

The VSV (−) DNA can be operably linked to a promoter before or after insertion of foreign DNA. In certain embodiments, a transcriptional terminator is situated downstream of the VSV (−) DNA.

In another embodiment, a DNA sequence that can be transcribed to produce a ribozyme sequence is situated at the immediate 3′ end of the VSV (−) DNA, prior to the transcriptional termination signal, so that upon transcription a self-cleaving ribozyme sequence is produced at the 3′ end of the antigenomic RNA, which ribozyme sequence will autolytically cleave (after a U) this fusion transcript to release the exact 3′ end of the VSV antigenomic (+) RNA. Any ribozyme sequence known in the art may be used, as long as the correct sequence is recognized and cleaved. In a preferred aspect, hepatitis delta virus (HDV) ribozyme is used (Perrotta and Been, 1991, Nature 350:434-436; Pattnaik et al., 1992, Cell 69:1011-1020).

An example of a VSV(—) DNA for use, for insertion of foreign DNA, can thus comprises (in 5′ to 3′ order) the following operably linked components: the T7 RNA polymerase promoter, VSV (−) DNA, a DNA sequence that is transcribed to produce an HDV ribozyme sequence (immediately downstream of the VSV (−) DNA), and a T7 RNA polymerase transcription termination site.

Examples of plasmids that can be used are, pVSVFL(+) or pVSVSS1.

In certain embodiments of the compositions and methods disclosed herein, the recombinant VSV particle, lacks a functional VSV G gene and encodes a coronavirus spike (S) glycoprotein, or a fragment or derivative thereof. In certain embodiments, VSV particles lacking a functional VSV G gene may result from any alteration or disruption of the VSV G gene, and/or expression of a poorly functional or nonfunctional VSV glycoprotein, or combinations thereof. By way of example, the VSV G gene can be deleted, but any mutation of the gene that alters the host range specificity of VSV or otherwise eliminates the function of the VSV glycoprotein can be employed. In certain embodiments, recombinant VSV particles can be generated which lack a functional glycoprotein or corresponding gene and express instead at least one protein or peptide of a coronavirus.

In certain embodiments, a coronavirus S protein can replace the endogenous VSV G protein in the recombinant VSV particle, or can be expressed as a fusion with the endogenous VSV G protein, or can be expressed in addition to the endogenous VSV G protein either as a fusion or nonfusion protein. For example, the G gene of VSV in the VSV (−) DNA of plasmid pVSVFL(+) can be excised and replaced, by cleavage at the NheI and MluI sites flanking the G gene and insertion of the desired sequence. In other embodiments, a coronavirus spike (S) protein is expressed as a fusion protein comprising the cytoplasmic domain (and, optionally, also the transmembrane region) of the VSV G protein. In certain embodiments, a coronavirus spike (S) protein forms a part of the VSV envelope and, thus, is surface-displayed in the VSV particle.

In certain embodiments, the VSV G glycoprotein is replaced by a coronavirus spike (S) glycoprotein, or a fragment or derivative thereof, wherein said coronavirus S glycoprotein, fragment or derivative is capable of mediating infection of a target cell.

Also provided is a recombinant VSV particle wherein (i) the VSV G glycoprotein is replaced by a coronavirus S glycoprotein or a fragment or a derivative thereof, wherein said S glycoprotein, fragment or derivative is capable of mediating infection of a target cell and wherein (ii) the recombinant VSV particle comprises a reporter protein or a nucleic acid molecule encoding the reporter protein. The nucleic acid sequence encoding the reporter protein may be inserted between the nucleic acid sequence encoding the coronavirus S glycoprotein and the nucleic acid sequence encoding VSV L protein.

In certain embodiments, foreign DNA is inserted into an intergenic region, or a portion of the VSV (−) DNA that is transcribed to form the noncoding region of a viral mRNA. In certain embodiments, the foreign DNA is inserted into a coding region of the VSV genome that is non-essential to the virus's development, growth and/or functions required to act as a vaccine. In certain embodiments, the VSV G gene is disrupted. In certain embodiments, the foreign DNA insertion does not disrupt the G gene or VSV G protein function.

### Foreign Proteins

Sources for the foreign protein can include any immunogen suitable for protecting a subject against an infectious disease, including but not limited to microbial, bacterial, protozoal, parasitic and viral diseases. Such infectious agent immunogens can include, but are not limited to, immunogens from Coronaviridae including coronaviruses such as the Severe Acute Respiratory Syndrome (SARS) coronavirus (e.g., SARS-CoV and SARS-CoV-2), and TGE virus (swine).

Coronaviruses form enveloped and spherical particles of 80-160 nm in diameter. They contain a positive-sense, non-segmented, single-stranded RNA (ssRNA) genome of 27-32 kb in size. The 5′-terminal two-thirds of the genome encodes polyproteins, pp1a and pp1ab. The 3′ terminus encodes structural proteins, including envelope glycoproteins spike (S), envelope (E), membrane (M) and nucleocapsid (N). The genomic RNA can associate with the N protein. The coronavirus M protein can interact with a cis-acting genomic RNA sequence. One or more structural proteins can be modified to comprise all or part of the intracellular region of the coronavirus M protein (for example, the C-terminal endodomain known to interact with the N protein), or a portion thereof containing the nucleic acid binding site, and the modified carrier virus genome comprises the cis-acting element that interacts with the M protein.

Coronavirus entry into host cells is mediated by the transmembrane spike (S) glycoprotein (also referred to as “spike glycoprotein”, “S glycoprotein”, “S protein” or “spike protein”) which is the main target of anti-viral neutralizing antibodies and is the focus of therapeutic and vaccine design in this disclosure. S glycoprotein forms homotrimers protruding from the viral surface. S glycoprotein comprises two functional subunits responsible for binding to the host cell receptor (S1 subunit) and fusion of the viral and cellular membranes (S2 subunit). For many coronaviruses, including SARS-CoV and SARS-CoV-2, S glycoprotein is cleaved at the boundary between the S1 and S2 subunits, which remain non-covalently bound in the prefusion conformation. The distal S1 subunit comprises the receptor-binding domain(s) (RBD) and contributes to stabilization of the prefusion state of the membrane-anchored S2 subunit that contains the fusion machinery. S is further cleaved by host proteases at the ST site located immediately upstream of the fusion peptide. This cleavage has been proposed to activate the protein for membrane fusion via conformational changes. Walls et al., Cell, published online Mar. 9, 2020; available at doi.org/10.1016/j.cell.2020.02.058.

SARS-CoV and SARS-CoV-2 interact directly with angiotensin-converting enzyme 2 (ACE2) to enter target cells and transmembrane serine protease 2 (TMPRSS2) may be of use for S protein priming (Hoffmann et al., Cell, 2020, 181:1-10; available at doi.org/10.1016/j.cell.2020.02.052). SARS-S and SARS-2-S share 76% amino acid identity. The receptor binding domain (RBD) in the S glycoprotein is the most variable part of the coronavirus genome. Six RBD amino acids have been shown to be critical for binding to ACE2 receptors and for determining the host range of SARS-CoV-like viruses. They are Y442, L472, N479, D480, T487 and Y4911 in SARS-CoV, which correspond to L455, F486, Q493, S494, N501 and Y505 in SARS-CoV-2 (Andersen et al., Nature Medicine, 2020).

In certain embodiments of the disclosure, the VSV particles comprise the SARS-CoV-2 S glycoprotein or a fragment or derivative thereof, wherein said S glycoprotein, fragment or derivative is capable of mediating infection of a target cell. In various embodiments, the S glycoprotein may be a full-length SARS-CoV-2 S glycoprotein (comprising or consisting of SEQ ID NO: 1) or a fragment or derivative thereof that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% amino acid sequence identity to SEQ ID NO: 1. In certain embodiments, the full-length SARS-CoV-2 S glycoprotein may be encoded by a codon optimized polynucleotide sequence. In various embodiments, the codon optimized polynucleotide sequence encoding the full-length SARS-CoV-2 S glycoprotein may comprise or consist of the polynucleotide sequence of SEQ ID NO: 2 or a fragment or derivative thereof that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% polynucleotide sequence identity to SEQ ID NO: 2.

In certain embodiments of the disclosure, the VSV particles comprise a fragment or derivative of the SARS-CoV-2 S glycoprotein. In certain embodiments the fragment or derivative of the SARS-CoV-2 S glycoprotein are functional fragments or derivatives.

In certain embodiments, the fragment or derivative of the SARS-CoV-2 S glycoprotein results in a more fusogenic recombinant VSV particle as compared to a recombinant VSV expressing a full-length wild-type SARS-CoV-2 spike protein inserted in the same location of the VSV genome. In certain embodiments, the fragment or derivative of the SARS-CoV-2 S glycoprotein results in a more lytic recombinant VSV particle as compared to a recombinant VSV expressing a full-length wild-type SARS-CoV-2 spike protein inserted in the same location of the VSV genome. In certain embodiments, the fragment or derivative of the SARS-CoV-2 S glycoprotein is not derived from a SARS-CoV-1 S glycoprotein.

The wild-type coronavirus S glycoprotein comprises an S1 subunit that facilitates binding of the coronavirus to cell surface proteins. Without wishing to be bound by theory, the S1 subunit of the wildtype S glycoprotein controls which cells are infected by the coronavirus. The wild-type S glycoprotein also comprises a S2 subunit, which is a transmembrane subunit that facilitates viral and cellular membrane fusion. In the various aspects and embodiments described herein, a fragment or derivative of SARS-CoV-2 S glycoprotein can comprise the S1 subunit of the SARS-CoV-2 S glycoprotein (i.e., amino acids 14-684 of SEQ ID NO: 1), or the S2 subunit of the SARS-CoV-2 S glycoprotein, or a fragment or derivative that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% amino acid sequence identity to the S1 subunit of the SARS-CoV-2 S glycoprotein or the S2 subunit of the SARS-CoV-2 S glycoprotein.

The wild-type coronavirus S glycoprotein comprises a receptor binding domain (RBD) that facilitates binding of the coronavirus to its receptor on the host cell. The RBD of the SARS-CoV-2 spike (S) glycoprotein is described, e.g., in Anderson et al., Nature Medicine, 2020 (available at doi.org/10.1038/s41591-020-0820-9). In the various aspects and embodiments described herein, a fragment or derivative of SARS-CoV-2 S glycoprotein can comprise the RBD of the SARS-CoV-2 S glycoprotein (i.e., amino acids 319-541 of SEQ ID NO: 1), or a fragment or derivative that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to the RBD of the SARS-CoV-2 S glycoprotein.

In certain embodiments, the SARS-CoV-2 S glycoprotein fragment or derivative lacks one or more C-terminal residues of the full-length SARS-CoV-2 S glycoprotein. For example, the SARS-CoV-2 S glycoprotein fragment may lack 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of the C-terminal residues of the SARS-CoV-2 S glycoprotein. In certain embodiments, the SARS-CoV-2 S glycoprotein fragment or derivative lacks the 19 C-terminal residues of the SARS-CoV-2 S glycoprotein. In some embodiments, SARS-CoV-2 S glycoprotein amino acids that have been removed are replaced by a VSV G protein sequence (SEQ ID NO: 15). In certain embodiments, the SARS-CoV-2 S glycoprotein fragment or derivative may consist of the amino acid sequence of SEQ ID NO: 3, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 3. In certain embodiments, the SARS-CoV-2 S glycoprotein fragment or derivative may be encoded by a codon optimized nucleotide sequence. In various embodiments, SARS-CoV-2 S glycoprotein fragment or derivative may be encoded by the polynucleotide sequence of SEQ ID NO: 4 or a sequence that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% polynucleotide sequence identity to SEQ ID NO: 4.

In certain embodiments, the SARS-CoV-2 S glycoprotein derivative is a fusion between a SARS-CoV-2 S glycoprotein, or a fragment or derivative thereof, and a protein the enables viral entry. In certain embodiments, the SARS-CoV-2 S glycoprotein derivative is a fusion between a SARS-CoV-2 S glycoprotein, or a fragment or derivative thereof, and a non-SARS-CoV-2 fusogen or a fragment or derivative thereof. In certain embodiments, the fusion protein is a fusion between a SARS-CoV-2 S glycoprotein, or a fragment or derivative thereof and a cytoplasmic portion of a non-SARS-CoV-2 fusogen or a fragment or derivative thereof. Non-limiting examples of fusogens used in the fusion molecules include, for example, coronavirus fusogens (e.g., from SARS-CoV-1 or MERS-CoV), fusogens from VSV or other vesiculoviruses or other viruses from the Rhabdoviridae family, viruses from the Retroviridae family (e.g., human immunodeficiency virus (HIV), murine leukemia virus (MLV), Avian sarcoma leukosis virus (ASLV), Jaagsiekte sheep retrovirus (JSRV)), viruses from the Paramyxoviridae family (e.g., parainfluenza virus 5 (PIVS)), viruses from the Herpesviridae family (e.g., herpes simplex virus (HSV)), viruses from the Togaviridae family (e.g., Semliki Forest virus (SFV), Rubella virus), viruses from the Flaviviridae family (e.g., tick-borne encephalitis virus (TBE), Dengue virus), viruses from the Orthomyxoviridae family (e.g., influenza virus), viruses from the Arenaviridae family (e.g., lymphocytic choriomenengitis virus (LCMV), Lassa fever virus (LASV)), viruses from the Bunyaviridae family (e.g., Uukuniemi Virus (UUKV)), viruses from the Filoviridae family (e.g., Ebola virus (EBOV)), viruses from the Poxviridae family (e.g., Vaccinia virus (VV)), viruses from the Asfaviridae family (e.g., African swine fever virus (ASFV)), viruses from the Arteriviridae family (e.g., porcine reproductive and respiratory syndrome virus (PRRSV)), viruses from the Bornaviridae family (e.g., Borna disease virus (BDV)), viruses from the Hepadnaviridae family (e.g., Hepatitis B virus (HBV)), and viruses from Hantaviridae family (e.g., Andes virus). In certain embodiments, the SARS-CoV-2 S glycoprotein derivative is a fusion between a SARS-CoV-2 S glycoprotein, or a fragment or derivative thereof, and a coronavirus spike protein or a fragment or derivative thereof.

In certain embodiments, the fusion protein is a fusion between a SARS-CoV-2 S glycoprotein, or a fragment or derivative thereof, and a VSV glycoprotein G protein or a fragment or derivative thereof. In certain embodiments, the fusion protein is a fusion between a SARS-CoV-2 S glycoprotein, or a fragment or derivative thereof, and a cytoplasmic portion of the VSV G glycoprotein or a fragment or derivative thereof. In some embodiments, the fusion protein is a fusion between a SARS-CoV-2 S glycoprotein, or a fragment or derivative thereof, and the VSV G cytoplasmic tail sequence (KLKHTKKRQIYTDIEMNRLGK (SEQ ID NO: 15)). In certain embodiments, the SARS-CoV-2 the fusion protein may comprise or consist of the amino acid sequence of SEQ ID NO: 5, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 5. In certain embodiments, the SARS-CoV-2 fusion protein may be encoded by a codon optimized nucleotide sequence. In various embodiments, the codon optimized polynucleotide sequence encoding the SARS-CoV-2 the fusion protein may comprise or consist of the polynucleotide sequence of SEQ ID NO: 6 or a fragment or derivative thereof that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% polynucleotide sequence identity to SEQ ID NO: 6.

In certain embodiments, the SARS-CoV-2 S glycoprotein derivative, or fragments thereof, differs in amino acid sequence from the reference peptide or polypeptide (e.g., wild-type SARS-CoV-2 spike protein) by the insertion, deletion, and/or substitution of one or more amino acids, but retains at least one biological activity of such reference peptide or polypeptide (e.g., the ability to mediate cell infection by a virus, the ability to mediate membrane fusion, the ability to be bound by a specific antibody or to promote an immune response, etc.) In certain embodiments, the derivative, or fragment thereof, of the SARS-CoV-2 S glycoprotein results in a more fusogenic recombinant VSV particle as compared to a recombinant VSV expressing a full-length wild-type SARS-CoV-2 spike protein inserted in the same location of the VSV genome. In certain embodiments, the derivative, or fragment thereof, of the SARS-CoV-2 S glycoprotein results in a more lytic recombinant VSV particle as compared to a recombinant VSV expressing a full-length wild-type SARS-CoV-2 spike protein inserted in the same location of the VSV genome. In certain embodiments, the SARS-CoV-2 S glycoprotein derivative, or fragment thereof, may comprise or consist of an insertion, deletion, and/or substitution of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 residues of the SARS-CoV-2 S glycoprotein. Non-limiting examples of amino acids for potential deletion include, e.g., a tyrosine at position (145), an asparagine at position (679), a serine at position (680), proline at position (681), an arginine at position (682), an arginine at position (683), an alanine at position (684), and/or an arginine at position (685), positions as denoted in SEQ ID NO: 1, or the equivalent amino acid residue in a mutant SARS-CoV-2 S glycoprotein sequence. Non-limiting examples of amino acids for potential substitution include, e.g., a leucine changed to a phenylalanine at position (5) a tyrosine changed to an asparagine at position (28), a threonine changed to an isoleucine at position (29), a histidine changed to a tyrosine at position (49), a leucine changed to a phenylalanine at position (54), an asparagine changed to a lysine at position (74), a glutamic acid changed to an aspartic acid at position (96), an aspartic acid changed to an asparagine at position (111), a phenylalanine changed to a leucine at position (157), a glycine changed to a valine at position (181), a serine changed to a tryptophan at position (221), a serine changed to an arginine at position (247), an alanine changed to a threonine at position (348), an arginine changed to an isoleucine at position (408), a glycine changed to a serine at position (476), a valine changed to an alanine at position (483), a histidine changed to a glutamine at position (519), an alanine changed to a serine at position (520), an aspartic acid changed to an asparagine at position (614), an aspartic acid changed to a glycine at position (614), an asparagine changed to an isoleucine at position (679), a serine change to a leucine at position (680), an arginine changed to a glycine at position (682), an arginine changed to a serine at position (683), an arginine changed to a glutamine at position (685), an arginine changed to a serine at position (685), a phenylalanine changed to a cysteine at position (797), an alanine changed to a valine at position (930), an aspartic acid changed to a tyrosine at position (936), an alanine changed to a valine at position (1078), an aspartic acid changed to a histidine at position (1168), and/or an aspartic acid changed to a histidine at position (1259), positions as denoted in SEQ ID NO: 1, or the equivalent amino acid residue in a mutant SARS-CoV-2 S glycoprotein sequence. See Becerra-Flores and Cardozo, “SARS-CoV-2 viral spike G614 mutation exhibits higher case fatality rate,” The International Journal of Clinical Practice, published online May 6, 2020; Eaaswarkhanth et al., “Could the D614G substitution in the SARS-CoV-2 spike (S) protein be associated with higher COVID-19 mortality?” International Journal of Infectious Diseases, 96: July 2020, Pages 459-460; Tang et al., “The SARS-CoV-2 Spike Protein D614G Mutation Shows Increasing Dominance and May Confer a Structural Advantage to the Furin Cleavage Domain,” Preprints 2020, 2020050407 (doi: 10.20944/preprints202005.0407.v1); Hansen et. al., “Studies in humanized mice and convalescent humans yield a SARS-CoV-2 antibody cocktail” Science, published online Jun. 15, 2020; Lokman et al., “Exploring the genomic and proteomic variations of SARS-CoV-2 spike glycoprotein: A computational biology approach”, Infection, Genetics and Evolution: Journal of Molecular Epidemiology and Evolutionary Genetics in Infectious Diseases, 2020 June; 84:104389. DOI: 10.1016/j.meegid.2020.104389, each of which incorporated herein by reference in their entirety for all intended purposes. Additional non-limiting examples of amino acid residue positions for insertion, deletion, and/or substitution include those as listed in Tables 2 and 3 (amino acid residue positions are denoted using SEQ ID NO: 1 as a reference sequence, which can be used as a reference for identifying the equivalent amino acid residue in any SARS-CoV-2 S glycoprotein sequence (same as above); references in Table 2 are incorporated herein by reference in their entirety for all intended purposes). Each residue modification listed in Table 2 can separately be used alone or in combination with others to generate variants of the virus.

TABLE 2
Non-Limiting Examples of Amino Acid Residue Positions for Insertion,
Deletion, and/or Substitution to Generate Variants of the Virus

| | | | |
|---|---|---|---|
| Mutations | Location | Phenotypes | References |

COVID VARIANT: B.1.1.7 lineage, (20I/501Y.V1 or VOC 202012/01)
Origin: UK
hCoV-19/England/SHEF-10C8326/2021

| | | | |
|---|---|---|---|
| N501Y | RBD | One of the six key amino | Horby P, Huntley C, Davies N, Edmunds J, |
| | | acids interacting with | Ferguson N, Medley G, et al. NERVTAG paper |
| | | ACE-2 receptor. | on COVID-19 variant of concern B.1.1.7 (2021) |
| | | Associated with increased | [www.gov.uk/government/publications/nervtag- |
| | | transmissibility (more | paper-on-covid-19-variant-ofconcern-b117] |
| | | efficient/rapid | Accession number: SAMN17373206 |
| | | transmissibility). | |
| 69-70 | | Potential conformational | Wu K, Werner A P, Moliva J I, et al. mRNA- |
| deletion | | change in spike protein. | 1273 vaccine induces neutralizing antibodies |
| | | Reduced sensitivity to | against spike mutants from global SARS-CoV- |
| | | neutralizing antibodies. | 2 variants. [Preprint Posted Jan. 25, 2021] |
| | | Associated with increased | GenBank: MW487270.1 |
| | | transmissibility (more | |
| | | efficient/rapid | |
| | | transmissibility). | |
| P681H | Near | Associated with increased | Xie X, Zou J, Fontes-Garfias C R, et al. |
| | S1/S2 | transmissibility (more | Neutralization of N501Y mutant SARS-CoV-2 |
| | furin | efficient/rapid | by BNT162b2 vaccine-elicited sera. [Preprint |
| | cleavage | transmissibility). | Posted Jan. 7, 2021] |
| | site | | Greaney A J, Loes A N, Crawford K H D, et al. |
| | | | Comprehensive mapping of mutations to the |
| | | | SARS-CoV-2 receptor-binding domain that |
| | | | affect recognition by polyclonal human serum |
| | | | antibodies. [Preprint Posted Jan. 4, 2021] |
| | | | Severe acute respiratory syndrome coronavirus |
| | | | 2 isolate SARS-CoV-2/human/USA/NYI.B1- |
| | | | 7.01-21/2021, complete genome |
| Y144 del | | | Weisblum Y, Schmidt F, Zhang F, et al. Escape |
| | | | from neutralizing antibodies by SARS-CoV-2 |
| | | | spike protein variants [eLife 2020; 9: e61312] |
| A570D | | | |
| T716I | | | |
| S982A | | | |
| D1118H | | | |

COVID VARIANT: B.1.351 (20H/501Y.V2)
Origin: South Africa

| | | | |
|---|---|---|---|
| K417N | RBD | Resistant to neutralizing | Weisblum Y, Schmidt F, Zhang F, et al. Escape |
| | | antibodies. | from neutralizing antibodies by SARS-CoV-2 |
| | | | spike protein variants [eLife 2020; 9: e61312] |
| | | | hCoV-19/Belgium/AZDelta05413-2105R/2021 |
| E484K | RBD | Resistant to neutralizing | Resende P C, Bezerra J F, de Vasconcelos R H T, |
| | | antibodies. E484K may | at al. Spike E484K mutation in the first SARS- |
| | | affect neutralization by | CoV-2 reinfection case confirmed in Brazil, |
| | | some polyclonal and | 2020external icon. [Posted on |
| | | mAb, potentially by | www.virological.orgextemal icon on Jan. 10, 2021] |
| | | disrupting the | |
| | | immunodominant B cell | |
| | | epitope, and is thought to | |
| | | be the mutation that drives | |
| | | immune escape. | |
| N501Y | RBD | Resistant to neutralizing | |
| | | antibodies, increased | |
| | | transmissibility. | |
| D614G | | | |
| A701V | | | |
| L18F | NTD | | |
| D80A | NTD | | |
| D215G | NTD | | |
| L242-244 | NTD | | |
| del | | | |
| R246I | NTD | Disrupts N5-loop (large, | |
| | | solvent exposed loop in | |
| | | NTD) and displaces the | |
| | | loop | |

COVID VARIANT: P.1 lineage (B1.1.28.1 or 20J/501.V3, 484K.V2)
Origin: Brazil

| | | | |
|---|---|---|---|
| K417T | RBD | Altered transmissibility | Resende P C, Bezerra J F, de Vasconcelos R H T, |
| E484K | RBD | and antigenic profile, | at al. Spike E484K mutation in the first SARS- |
| N501Y | RBD | which may affect ability | CoV-2 reinfection case confirmed in Brazil, |
| L18F | NTD | of Ab generated through | 2020extemal icon. [Posted on |
| T20N | NTD | previous natural infection | www.virological.orgextemal icon on |
| P26S | | or vaccination to | Jan. 10, 2021] |
| D138Y | | recognize and neutralize | hCo V-19/Brazil/RR-1087/2021 |
| R190S | | virus. | |
| D614G | | | |
| H655Y | | | |
| T1027I | | | |

COVID VARIANT: B.1.429 (CAL.20C, CA VUI)
Origin: California

S131
W152C
L452R
D614G

COVID VARIANT: B.1.2 lineage 20C-US

| | | |
|---|---|---|
| Q677H | Adjacent | Adrian A. Pater et al., Emergence and |
| | to furin | Evolution of a Prevalent New SARS-CoV-2 |
| | cleavage | Variant in the United States |
| | site | [doi.org/10.1101/2021.01.11.426287] |
| Other | | |
| mutations | | |
| in ORFs | | |

COVID VARIANT: B1.1.17

Weisblum Y, Schmidt F, Zhang F, et al. Escape
from neutralizing antibodies by SARS-CoV-2
spike protein variants [eLife 2020; 9: e61312]

COVID VARIANT: 20E (EU1)

A22V
D614G

COVID VARIANT: 20A.EU2

S477N
D614G

COVID VARIANT: N439K-D614G

N439K
D614G

COVID VARIANT: Mink Cluster 5 variant

| | | |
|---|---|---|
| H69 del | | |
| V70 del | | |
| Y453F | RBD | Increased binding affinity |
| | | for mink Ace2. |
| D614G | | |
| I692V | | |
| M1229I | | |

TABLE 3
Non-Limiting Examples of Variants of the Virus

| Variant Name | Mutations |
|---|---|
| VSV-SARS-CoV2-S_E484K | E484K |
| VSV-SARS-CoV2-S_B.1.351_NTD | L18F, D80A, D215G, 242-244 del, R246I, A701V |
| VSV-SARS-CoV2-S_B.1.351_RBD | K417N, E484K, N501Y, D614G |
| VSV-SARS-CoV2-S_B.1.351 | K417N, E484K, N501Y, D614G, L18F, D80A, D215G, 242-244 del, R246I, A701V |
| VSV-SARS-CoV2-S_B.1.1.7_RBD | N501Y, 69-70 del, P681H |
| VSV-SARS-CoV2-S_B.1.1.7 | N501Y, 69-70 del, P681H, Y144 del, A570D, T716I, S982A, D1118H |
| VSV-SARS-CoV2-S_B.1.1.28_RBD | K417T, E484K, N501Y |

In certain embodiments, the SARS-CoV-2 S glycoprotein derivative, or fragments thereof, differs in amino acid sequence from the reference peptide or polypeptide (e.g., wild-type SARS-CoV-2 spike protein) by changing a serine to an arginine at position (247), an aspartic acid to an asparagine at position (614), and/or an arginine to a glutamine at position (685), positions as denoted in SEQ ID NO: 1, or the equivalent amino acid residue in a mutant SARS-CoV-2 S glycoprotein sequence. In certain embodiments, the SARS-CoV-2 S glycoprotein derivative, or fragments thereof, differs in amino acid sequence from the reference peptide or polypeptide by changing a serine to an arginine at position (247). In certain embodiments, the SARS-CoV-2 S glycoprotein derivative, or fragments thereof, differs in amino acid sequence from the reference peptide or polypeptide by changing an aspartic acid to an asparagine at position (614). In certain embodiments, the SARS-CoV-2 S glycoprotein derivative, or fragments thereof, differs in amino acid sequence from the reference peptide or polypeptide by changing an arginine to a glutamine at position (685). In certain embodiments, the SARS-CoV-2 S glycoprotein derivative, or fragments thereof, differs in amino acid sequence from the reference peptide or polypeptide by changing a serine to an arginine at position (247) and an aspartic acid to an asparagine at position (614). In certain embodiments, the SARS-CoV-2 S glycoprotein derivative, or fragments thereof, differs in amino acid sequence from the reference peptide or polypeptide by changing a serine to an arginine at position (247) and an arginine to a glutamine at position (685). In certain embodiments, the SARS-CoV-2 S glycoprotein derivative, or fragments thereof, differs in amino acid sequence from the reference peptide or polypeptide by changing an aspartic acid to an asparagine at position (614) and an arginine to a glutamine at position (685). In certain embodiments, the SARS-CoV-2 S glycoprotein derivative, or fragments thereof, differs in amino acid sequence from the reference peptide or polypeptide by changing a serine to an arginine at position (247), an aspartic acid to an asparagine at position (614), and an arginine to a glutamine at position (685). In certain embodiments, the SARS-CoV-2 S glycoprotein derivative, or fragments thereof, result in a more lytic phenotype. In certain embodiments, the SARS-CoV-2 S glycoprotein fragment or derivative may comprise the amino acid sequence of SEQ ID NO: 20, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 20. In certain embodiments, the SARS-CoV-2 S glycoprotein fragment or derivative may be encoded by a codon optimized nucleotide sequence. In various embodiments, SARS-CoV-2 S glycoprotein fragment or derivative may be encoded by the polynucleotide sequence of SEQ ID NO: 21 or a sequence that has at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% polynucleotide sequence identity to SEQ ID NO: 21.

In certain embodiments, the SARS-CoV-2 S glycoprotein derivative, or fragments thereof, differs in amino acid sequence from the reference peptide or polypeptide (e.g., wild-type SARS-CoV-2 spike protein) by changing an asparagine to a tyrosine at position (501), a glutamic acid to a lysine at position (484), an aspartic acid to a glycine at position (614), and/or deletion of residues 69-70, positions as denoted in SEQ ID NO: 1, or the equivalent amino acid residue in a mutant SARS-CoV-2 S glycoprotein sequence. In certain embodiments, the SARS-CoV-2 S glycoprotein derivative, or fragments thereof, differs in amino acid sequence from the reference peptide or polypeptide by changing an asparagine to a tyrosine at position (501). In certain embodiments, the SARS-CoV-2 S glycoprotein derivative, or fragments thereof, differs in amino acid sequence from the reference peptide or polypeptide by changing a glutamic acid to a lysine at position (484). In certain embodiments, the SARS-CoV-2 S glycoprotein derivative, or fragments thereof, differs in amino acid sequence from the reference peptide or polypeptide by changing an aspartic acid to a glycine at position (614). In certain embodiments, the SARS-CoV-2 S glycoprotein derivative, or fragments thereof, differs in amino acid sequence from the reference peptide or polypeptide by deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501) and a glutamic acid to a lysine at position (484). In certain embodiments, the SARS-CoV-2 S glycoprotein derivative, or fragments thereof, differs in amino acid sequence from the reference peptide or polypeptide by changing an asparagine to a tyrosine at position (501) and an aspartic acid to a glycine at position (614). In certain embodiments, the SARS-CoV-2 S glycoprotein derivative, or fragments thereof, differs in amino acid sequence from the reference peptide or polypeptide by changing an asparagine to a tyrosine at position (501) and deletion of residues 69-70. In certain embodiments, the SARS-CoV-2 S glycoprotein derivative, or fragments thereof, differs in amino acid sequence from the reference peptide or polypeptide by changing a glutamic acid to a lysine at position (484) and an aspartic acid to a glycine at position (614). In certain embodiments, the SARS-CoV-2 S glycoprotein derivative, or fragments thereof, differs in amino acid sequence from the reference peptide or polypeptide by changing a glutamic acid to a lysine at position (484) and deletion of residues 69-70. In certain embodiments, the SARS-CoV-2 S glycoprotein derivative, or fragments thereof, differs in amino acid sequence from the reference peptide or polypeptide by changing an aspartic acid to a glycine at position (614) and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), a glutamic acid to a lysine at position (484), and an aspartic acid to a glycine at position (614). In certain embodiments, the SARS-CoV-2 S glycoprotein derivative, or fragments thereof, differs in amino acid sequence from the reference peptide or polypeptide by changing an asparagine to a tyrosine at position (501), changing a glutamic acid to a lysine at position (484), and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), changing an aspartic acid to a glycine at position (614), and deletion of residues 69-70. In certain embodiments, the SARS-CoV-2 S glycoprotein derivative, or fragments thereof, differs in amino acid sequence from the reference peptide or polypeptide by changing a glutamic acid to a lysine at position (484), changing an aspartic acid to a glycine at position (614), and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), changing a glutamic acid to a lysine at position (484), changing an aspartic acid to a glycine at position (614) and deletion of residues 69-70. In certain embodiments, the SARS-CoV-2 S glycoprotein fragment or derivative may comprise the amino acid sequence of SEQ ID NO: 22, or a sequence at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to the amino acid sequence of SEQ ID NO: 22.

In certain embodiments, the SARS-CoV-2 S glycoprotein derivative, or fragments thereof, differs in amino acid sequence from the reference peptide or polypeptide (e.g., wild-type SARS-CoV-2 spike protein) by inactivating the furin cleavage site within the spike protein. In certain embodiments, the SARS-CoV-2 S glycoprotein derivative, or fragments thereof, differs in amino acid sequence from the reference peptide or polypeptide (e.g., wild-type SARS-CoV-2 spike protein) by changing Q⁶⁷⁷TNSPRRARSV⁶⁸⁷, as denoted in SEQ ID NO: 1, or the equivalent amino acid residue in a mutant SARS-CoV-2 S glycoprotein sequence, to QTILRSV or to QTNSPGSASSV. In certain embodiments, the SARS-CoV-2 S glycoprotein derivative, or fragments thereof, result in a monobasic furin cleavage site in the S1/S2 interface (QTILRSV) or deletion of the furin cleavage site (QTNSPGSASSV) phenotype. In certain embodiments, the alteration to the furin cleavage site can lead to a spike stabilized pseudoparticles. See Hansen et. al., “Studies in humanized mice and convalescent humans yield a SARS-CoV-2 antibody cocktail” Science, published online Jun. 15, 2020, incorporated herein by reference in its entirety for all intended purposes.

Polynucleotide molecules encoding the SARS-CoV-2 S glycoprotein or a fragment or derivative thereof can comprise a consensus sequence and/or modification(s) for improved expression of the SARS-CoV-2 S glycoprotein or the fragment or derivative thereof. Modification can include codon optimization, the addition of a Kozak sequence or modified (e.g., optimized) Kozak sequence for increased translation initiation, and/or the addition of a signal peptide/leader sequence (e.g., an immunoglobulin signal peptide such as, e.g., IgE or IgG signal peptide). In certain embodiments, the Kozak sequence or modified (e.g., optimized) Kozak sequence is 3′ to the foreign gene. In certain embodiments, the Kozak sequence or modified (e.g., optimized) Kozak sequence is 5′ to the foreign gene. In certain embodiments, the Kozak sequence or modified (e.g., optimized) Kozak sequence is immediately 3′ to the foreign gene. In certain embodiments, the Kozak sequence or modified (e.g., optimized) Kozak sequence is immediately 5′ to the foreign gene.

In some embodiments, the SARS-CoV-2 S glycoprotein or a fragment or derivative thereof comprises a fusions or conjugate with a detection tag (e.g., HA tag, histidine tag, biotin), a reporter protein or a fragment thereof, dimerization/multimerization sequences, Fc, signaling sequences, etc. In some embodiments, the recombinant VSV particles described herein comprise, in addition to the SARS-CoV-2 S glycoprotein or a fragment or derivative thereof, a reporter protein or a fragment thereof, wherein said reporter protein or a fragment thereof is either encoded by the VSV particle genome or is included in it as a protein. Non-limiting examples of reporter proteins include, e.g., luciferases (including but not limited to, Renilla luciferase or a mutant thereof, (dCpG)Luciferase, NanoLuc reporter, firefly luciferase, MetLuc, Vibrio fischeri lumazine protein, Vibrio harveyi luminaze protein, inoflagellate luciferase, firefly luciferase YY5 mutant, firefly luciferase LGR mutant, firefly luciferase mutant E, and derivatives thereof) and fluorescent proteins (including but not limited to, green fluorescent protein (GFP) [e.g., Aequorea victoria GFP, Renilla muelleri GFP, Renilla reniformis GFP, Renilla ptilosarcus GFP], GFP-like fluorescent proteins, (GFP-like), enhanced green fluorescent protein (EGFP), yellow fluorescent protein (YFP) [e.g., Topaz, Venus, mCitrine, YPet, TagYFP, PhiYFP, ZsYellow1, mBanana], enhanced yellow fluorescent protein (EYFP), blue fluorescent protein (BFP) [e.g., EBFP2, Azurite, GFP2, GFP10, and mTagBFP], enhanced blue fluorescent protein (EBFP), cyan fluorescent protein (CFP) [e.g., mECFP, Cerulean, CyPet, AmCyan1, Midori-Ishi Cyan, TagCFP, mCFPmm, mTFP1 (Teal)], enhanced cyan fluorescent protein (ECFP), superfolder GFP, superfolder YFP, orange fluorescent protein [e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine], red fluorescent protein [e.g., mRuby, mApple, mStrawberry, AsRed2, mRFP1, JRed, mCherry, HcRedl, mRaspberry, dKeima-Tandem, HcRed-Tandem, mPlum, tdTomato, AQ143], small ultrared fluorescent protein, FMN-binding fluorescent protein, dsRed, qFP611, Dronpa, TagRFP, KFP, EosFP, IrisFP, Dendra, Kaede, KikGr1, emerald fluorescent protein, Azami Green, mWasabi, TagGFP, TurboGFP, AcGFP, ZsGreen, T-Sapphire, and derivatives thereof), β-galactosidase, β-glucuronidase, β-geo, and fragments thereof.

In some embodiments, the coronavirus S protein, fragment or derivative thereof is derived from SARS-CoV-2. In certain embodiments, the coronavirus S protein is a full-length SARS-CoV-2 S protein (e.g., a protein comprising or consisting of the amino acid sequence of SEQ ID NO: 1). In certain embodiments, the coronavirus S protein is a SARS-CoV-2 S protein lacking 19 C-terminal amino acids (e.g., a protein comprising or consisting of the amino acid sequence of SEQ ID NO: 3). In certain embodiments, the coronavirus S protein is a fusion protein between a SARS-CoV-2 S glycoprotein, or a fragment or derivative thereof, and the VSV G cytoplasmic tail sequence (e.g., a protein comprising or consisting of the amino acid sequence of SEQ ID NO: 5). In certain embodiments, the coronavirus S protein, fragment or derivative has at least 80% amino acid sequence identity to the amino acid sequence of SEQ ID NO: 1. In certain embodiments, the coronavirus S protein, fragment or derivative has at least 80% amino acid sequence identity to amino acids 14-684 of the amino acid sequence of SEQ ID NO: 1. In certain embodiments, the coronavirus S protein, fragment or derivative has at least 80% amino acid sequence identity to amino acids 319-541 of the amino acid sequence of SEQ ID NO: 1. In certain embodiments, the recombinant VSV particle comprises a VSV matrix (M) protein. In certain embodiments, the VSV matrix M protein comprises or consists of the amino acid sequence of SEQ ID NO: 9. In certain embodiments, the recombinant VSV particle comprises a mutant VSV M protein. In certain embodiments, the genome of the recombinant VSV encodes a mutant VSV M protein. In certain embodiments, the mutant M protein comprises a mutation at methionine (M) 51 (e.g., a change from methionine (M) to arginine (R)). In certain embodiments, the mutant VSV matrix M protein comprises or consists of the amino acid sequence of SEQ ID NO: 7.

### Production of Recombinant VSV Particles

The recombinant VSV particles described herein are produced by providing in an appropriate host cell: VSV (−) DNA, in which regions non-essential for replication have been inserted into or replaced by a foreign DNA comprising a sequence encoding a non-VSV immunogenic and/or antigenic protein or peptide (e.g., coronavirus S glycoprotein) or a fragment or derivative thereof and optionally other sequences discussed above, and recombinant sources of VSV N protein, P protein, L protein and any additional desired VSV protein (e.g., M protein and/or G glycoprotein). In certain embodiments, the production is preferably in vitro (e.g., in cell culture).

The host cell used for recombinant VSV production can be any cell in which VSVs grows. Non-limiting sources of host cells include, prokaryotic cells or a eukaryotic cells, vertebrate cells, mammalian cells, some insect (e.g., Drosophila) cells, primary cells (e.g., primary chick embryo fibroblasts), or cell lines (e.g., BHK (baby hamster kidney) cells, CHO (Chinese hamster ovary) cells, HeLA (human) cells, mouse L cells, Vero (monkey) cells, ESK-4, PK-15, EMSK cells, MDCK (Madin-Darby canine kidney) cells, MDBK (Madin-Darby bovine kidney) cells, 293 (human) cells, Hep-2 cells, Human Diploid Primary Cell Lines (e.g. WI-38 and MRCS cells), Monkey Diploid Cell Line (e.g. FRhL-Fetal Rhesus Lung cells), and Quasi-Primary Continues Cell Line (e.g. AGMK-African green monkey kidney cells), etc.).

The sources of N, P, and L proteins and any additional desired VSV protein (e.g., M protein and/or G glycoprotein) can be the same or can be different recombinant nucleic acid(s), encoding and capable of expressing these proteins in the host cell in which it is desired to produce recombinant VSVs. The nucleic acids encoding the N, P and L proteins and any additional desired VSV protein (e.g., M protein and/or G glycoprotein) can be obtained by any means available in the art. The VSV N, P, L, M and G-encoding nucleic acid sequences have been disclosed and can be used. For example, see Genbank accession no. J02428; Rose and Schubert, 1987, in The Viruses: The Rhabdoviruses, Plenum Press, NY, pp. 129-166. The sequences encoding the N, P and L genes can also be obtained, for example, from plasmid pVSVFL(+), deposited with the ATCC and assigned accession no. 97134, e.g., by PCR amplification of the desired gene (see also U.S. Pat. Nos. 4,683,202, 4,683,195 and 4,889,818; Gyllenstein et al., 1988, Proc. Natl. Acad. Sci. USA 85:7652-7656; Ochman et al., 1988, Genetics 120:621-623; Loh et al., 1989, Science 243:217-220). If a nucleic acid clone of any of the N, P, L, M or G genes is not already available, the clone can be obtained by use of standard recombinant DNA methodology. For example, the DNA may be obtained by standard procedures known in the art such as, e.g., by purification of RNA from VSV virions followed by reverse transcription and PCR (Mullis and Faloona, 1987, Methods in Enzymology 155:335-350). Alternatives include, but are not limited to, chemically synthesizing the gene sequence itself. Other methods are possible and within the scope of the disclosure.

Nucleic acids that encode fragments and derivatives of VSV N, P, L, M, and/or G genes, as well as fragments and derivatives of the VSV (−) DNA can also be used in the present disclosure, as long as such fragments and derivatives retain the requisite function (e.g., the ability to produce replication-competent or replication-deficient VSV particles which can be used in one or more methods described herein). In particular, derivatives can be made by altering sequences by substitutions, additions, or deletions. Furthermore, due to the inherent degeneracy of the genetic code, other DNA sequences which encode substantially the same or a functionally equivalent amino acid sequence may be used in the practice of the methods of the disclosure. Amino acid substitutions may be made on the basis of similarity in polarity, charge, solubility, hydrophobicity, hydrophilicity and/or the amphipathic nature of the residues involved.

The desired N/P/L/M/G-encoding nucleic acid can be inserted into an appropriate expression vector, i.e., a vector which contains the necessary elements for the transcription and translation of the inserted protein-coding sequence in the host cell in which it is desired to produce recombinant VSV particles, to create a vector that functions to direct the synthesis of the VSV proteins that will subsequently assemble with the VSV genomic RNA (e.g., produced in the host cell from antigenomic VSV (+) RNA produced, e.g., by transcription of the VSV (−) DNA).

A variety of vector systems may be utilized to express the N, P and L VSV proteins and any additional desired VSV protein (e.g., M and/or G), as well as to transcribe the VSV (−) DNA (e.g., comprising a foreign DNA), as long as the vector is functional in the host cell and compatible with any other vector present. The expression elements of vectors vary in their strengths and specificities. Any one of a number of suitable transcription and translation elements may be used, as long as they are functional in the host cell.

Standard recombinant DNA methods may be used to construct expression vectors containing DNA encoding the VSV proteins, and the VSV (−) DNA containing the foreign DNA, comprising appropriate transcriptional/translational control signals (see, e.g., Sambrook et al., 1989, supra, and methods described hereinabove). Expression may be controlled by any promoter/enhancer element known in the art. Promoters which may be used to control expression can be constitutive or inducible. In a specific embodiment, the promoter is an RNA polymerase promoter.

Transcription termination signals (downstream of the gene), and selectable markers are preferably also included in the expression vector. In addition to promoter sequences, expression vectors for the N, P, L, and any additionally desired VSV proteins, as well as any coronavirus proteins, may contain specific initiation signals for efficient translation of the inserted sequences, e.g., a ribosome binding site.

Specific initiation signals maybe required for efficient translation of the protein coding sequences. These signals include the ATG initiation codon and adjacent sequences. In cases where the entire N, P, L, or other (e.g., M and/or G) VSV gene, including its own initiation codon and adjacent sequences, are inserted into the appropriate vectors, no additional translational control signals may be needed. However, in cases where only a portion of the gene sequence is inserted, exogenous translational control signals, including the ATG initiation codon, must be provided. The initiation codon must furthermore be in phase with the reading frame of the protein coding sequences to ensure translation of the entire insert. These exogenous translational control signals and initiation codons can be of a variety of origins, both natural and synthetic.

In a specific embodiment, a recombinant expression vector provided by the disclosure, encoding an N, P, L, and/or other (e.g., M and/or G) protein or functional derivative thereof, comprises the following operatively linked components: a promoter which controls the expression of proteins (e.g., the N, P, L, and/or other VSV protein (for example, M and/or G), a coronavirus protein (e.g., a spike glycoprotein such as the SARS-CoV-2 spike glycoprotein), or a fragment or derivative thereof, a translation initiation signal, a DNA sequence encoding the VSV protein or functional fragment or derivative thereof, and a transcription termination signal. In certain embodiments, the above components are present in 5′ to 3′ order as listed above. In certain embodiments, genes encoding the M protein, G proteins, and/or coronavirus S glycoprotein or a fragment or derivative thereof are interspersed between the N, P, and/or L proteins. In certain embodiments, genes for the M protein, G protein, and/or coronavirus S glycoprotein or a fragment or derivative thereof are between the genes for P and L proteins (see FIG. 1). In certain embodiments, the N, P, and L proteins or functional fragment or derivative thereof are not present in the 5′ to 3′ order as listed above. In certain embodiments, the order is altered (e.g., to attenuate the recombinant VSV).

In certain embodiments, the genes encoding the N, P, L, and other (e.g., M and/or G) VSV proteins are inserted downstream of the T7 RNA polymerase promoter from phage T7 gene 10, situated with an A in the −3 position. A T7 RNA polymerase terminator and a replicon can be also included in the expression vector. T7 RNA polymerase can be provided to transcribe the VSV protein sequence. The T7 RNA polymerase can be produced from a chromosomally integrated sequence or an episomal vector. In certain embodiments, T7 RNA polymerase can be provided by intracellular expression from a recombinant vaccinia virus vector encoding the T7 RNA polymerase. In certain embodiments, the N, P, L, and/or other (e.g., M and/or G) VSV proteins are each encoded by a DNA sequence operably linked to a promoter in an expression plasmid, containing the necessary regulatory signals for transcription and translation of the encoded proteins. Such an expression plasmid preferably includes a promoter, the coding sequence, and a transcription termination/polyadenylation signal, and optionally, a selectable marker (e.g., β-galactosidase).

In certain embodiments, the N, P, L, and/or other (e.g., M and/or G) proteins can be encoded by the same or different plasmids, or a combination thereof. In other embodiments, one or more of the N, P, L, and other (e.g., M and/or G) VSV proteins can be expressed intrachromosomally.

The cloned sequences comprising the VSV (−) DNA containing the foreign DNA, and the cloned sequences comprising sequences encoding the VSV and foreign proteins can be introduced into the desired host cell by any method known in the art, e.g., transfection, electroporation, infection (when the sequences are contained in, e.g., a viral vector), microinjection, etc. In certain embodiments, a transfection facilitating reagent is added to increase DNA uptake by cells. Many of these reagents are known in the art (e.g., calcium phosphate; Lipofectace (Life Technologies, Gaithersburg, Md.), and Effectene (Qiagen, Valencia, Calif.) are non-limiting examples).

In certain embodiments, DNA comprising VSV (−) DNA containing foreign DNA encoding a coronavirus S glycoprotein or a fragment or derivative thereof, operably linked to an RNA polymerase promoter (e.g., a bacteriophage RNA polymerase promoter); DNA encoding N, operably linked to the same RNA polymerase promoter; DNA encoding P, operably linked to the same polymerase promoter; and DNA encoding L, operably linked to the same polymerase promoter; are all introduced (e.g., by transfection) into the same host cell, in which host cell the RNA polymerase has been cytoplasmically provided. In certain embodiments, the RNA polymerase is cytoplasmically provided by expression from a recombinant virus vector that replicates in the cytoplasm and expresses the RNA polymerase, most preferably a vaccinia virus vector, that has been introduced (e.g., by infection) into the same host cell. Cytoplasmic provision of RNA polymerase can be used, as this will result in cytoplasmic transcription and processing, of the VSV (−) DNA comprising the foreign DNA and of the N, P, L, and other (e.g., M and/or G protein) VSV proteins, avoiding splicing machinery in the cell nucleus, and, thereby, maximizing proper processing and production of N, P, L, and other (e.g., M and/or G protein) VSV proteins, and resulting assembly of the recombinant VSVs. Vaccinia virus vectors also cytoplasmically provide enzymes for processing (capping and polyadenylation) of mRNA, facilitating proper translation. In a most preferred aspect, T7 RNA polymerase promoters are employed, and a cytoplasmic source of T7 RNA polymerase is provided by also introducing into the host cell a recombinant vaccinia virus vector encoding T7 RNA polymerase into the host cell. Such vaccinia virus vector can be obtained by well-known methods. In certain embodiments, a recombinant vaccinia virus vector such as vTF7-3 (Fuerst et al., 1986, Proc. Natl. Acad. Sci. U.S.A. 83:8122-8126) can be used.

In certain embodiments, the recombinant VSV particles described herein can be produced by co-transfecting host cells with five plasmids: 1) a plasmid comprising DNA that can be transcribed to encode VSV antigenomic (+) RNA (complementary to the VSV genome), wherein the DNA encodes VSV N, P, L, and M, or fragments or derivatives thereof, and DNA encoding the foreign protein or peptide, 2) a plasmid comprising a recombinant source of VSV N protein, 3) a plasmid comprising a recombinant source of VSV P protein, 4) a plasmid comprising a recombinant source of VSV L protein, and 5) a plasmid comprising a recombinant source of VSV G glycoprotein; under conditions such that the DNA is transcribed to produce the antigenomic RNA, and a VSV is produced that contains genomic RNA complementary to the antigenomic RNA produced and foreign RNA, which is not naturally a part of the VSV genome, from the DNA. Plasmids 2-5 help to enhance the efficiency of virus rescue. The cells may be passed several times to ensure the viral preparation is clean of VSV G glycoprotein. In some embodiments, the G glycoprotein is labeled with a marker (e.g., GFP) that helps determine when the viral preparation is free of VSV G glycoprotein.

In other embodiments, the RNA polymerase (e.g., T7 RNA polymerase) can be provided by use of a host cell that expresses T7 RNA polymerase from a chromosomally integrated sequence (e.g., originally inserted into the chromosome by homologous recombination), optionally constitutively, or that expresses T7 RNA polymerase episomally, from a plasmid.

In other embodiments, the VSV (−) DNA encoding a foreign protein or peptide (e.g., coronavirus S glycoprotein or a fragment or derivative thereof), operably linked to a promoter, can be transfected into a host cell that stably recombinantly expresses the N, P, L, and any other (e.g., M and/or G protein) VSV proteins from chromosomally integrated sequences.

The cells are cultured and recombinant VSV can be recovered, e.g., using standard methods. By way of example, and not limitation, after approximately 24 hours, cells and medium can be collected, freeze-thawed, and the lysates clarified to yield virus preparations. Alternatively, the cells and medium can be collected and simply cleared of cells and debris by low-speed centrifugation.

Confirmation that the appropriate foreign sequence is present in the genome of the recombinant VSV and directs the production of the desired protein(s) in an infected cell, can be performed. Standard procedures known in the art can be used for this purpose. By way of example, and not limitation, genomic RNA can be obtained from the VSV by SDS phenol extraction from virus preparations, and can be subjected to reverse transcription (and/or PCR), followed by e.g., sequencing, Southern hybridization using a probe specific to the foreign DNA, or restriction enzyme mapping, etc. The virus can be used to infect host cells, which can then be assayed for expression of the desired protein by standard immunoassay techniques using an antibody to the protein (e.g., Western blotting), or by assays based on functional activity of the protein. Other techniques are known in the art and can be used.

### Large Scale Growth and Purification of the Recombinant Viruses

VSVs are used as an example in the disclosure below, and this disclosure can also be used for other rhabdoviruses and vesiculoviruses.

A non-limiting example of a large-scale production of a recombinant VSV virus following plaque-purification is presented below. Virus from a single plaque (˜10⁵ pfu) is recovered and used to infect ˜10⁷ cells (e.g., BHK cells), to yield, generally, 10 ml at a titer of 10⁹-10¹⁰ pfu/ml for a total of approximately 10¹¹ pfu. Infection of ˜10¹² cells can then be carried out (with a multiplicity of infection of e.g., 0.1), and the cells can be grown in suspension culture, large dishes, or roller bottles by standard methods known to those in the art.

Virus for vaccine preparations can then be collected from culture supernatants, and the supernatants clarified to remove cellular debris. If desired, one method of isolating and concentrating the virus that can be employed is by passage of the supernatant through a tangential flow membrane concentration. The harvest can be further reduced in volume by pelleting through a glycerol cushion and by concentration on a sucrose step gradient. An alternate method of concentration is affinity column purification (Daniel et al., 1988, Int. J. Cancer 41:601-608). However, other methods can also be used for purification (see, e.g., Arthur et al., 1986, J. Cell. Biochem. Suppl. 10A:226), and any possible modifications of the above procedure will be readily recognized by one skilled in the art. Purification should be as gentle as possible, so as to maintain the integrity of the virus particle.

Immunogenic and/or Antigenic Compositions and Vaccines and Administration

In one aspect, the disclosure provides a recombinant VSV particles that express a foreign protein (e.g., a coronavirus protein) to be used as an antigen in an immunogenic and/or antigenic composition or vaccine.

In certain embodiments, an immunogenic and/or antigenic composition or vaccine is formulated such that the immunogen is one or several recombinant VSV particles, in which the foreign RNA in the genome directs the production of foreign protein in a host so as to elicit an immune (humoral and/or cell mediated) response in the host that is prophylactic or therapeutic. In an embodiment wherein the foreign protein displays the immunogenicity and/or antigenicity of an antigen of a pathogen (e.g., SARS-Cov-2), administration of the immunogenic and/or antigenic composition or vaccine is carried out to prevent or treat an infection by the pathogen and/or the resultant infectious disorder and/or other undesirable correlates of infection.

In a specific embodiment, the immunogenic and/or antigenic composition or vaccine comprises one or several recombinant VSV particles expressing a SARS-CoV-2 S glycoprotein, wherein the immunogenic and/or antigenic composition or vaccine is used for the treatment or prevention of a disease or disorder in a subject infected with SARS-CoV-2. In certain embodiments, the disease or disorder is COVID-19.

The recombinant VSV particles described herein for use as therapeutic or prophylactic live vaccines according to the disclosure maybe somewhat attenuated. Most available strains e.g., laboratory strains of VSV, may be sufficiently attenuated for use. Should additional attenuation be desired, e.g., based on pathogenicity testing in animals, attenuation may be achieved simply by laboratory passage of the recombinant VSVs (e.g., in BHK or any other suitable cell line). Generally, attenuated viruses are obtainable by numerous methods known in the art including, but not limited to, chemical mutagenesis, genetic insertion, deletion (Miller, 1972, Experiments in Molecular Genetics, Cold Spring Harbor Laboratory, Cold Spring Harbor, N.Y.) or recombination using recombinant DNA methodology (Maniatis et al., 1982, Molecular Cloning, A Laboratory Manual, Cold Spring Harbor Laboratory, Cold Spring Harbor, N.Y.), laboratory selection of natural mutants, etc.

In certain embodiments, the recombinant replication-competent VSV particles described herein can be inactivated (i.e., killed, rendered nonreplicable) prior to vaccine use, to provide a killed vaccine. Because the VSV envelope is immunogenic and/or antigenic, in an embodiment wherein one or more foreign proteins (e.g., an envelope glycoprotein of a virus other than a VSVs) is incorporated into the VSV envelope, such a virus, even in killed form, can be effective to provide an immune response against said foreign protein(s) in a host to which it is administered. In a specific embodiment, a multiplicity of foreign proteins, each displaying the immunogenicity or antigenicity of an envelope glycoprotein of a different virus, are present in the recombinant VSV particle.

The inactivated recombinant viruses described herein differ from defective interfering particles in that, prior to inactivation the virus is replication-competent (i.e., it encodes all the VSV proteins necessary to enable it to replicate in an infected cell). Thus, since the virus is originally in a replication-competent state, it can be propagated and grown to large amounts prior to inactivation, to provide a large amount of killed virus for use in vaccines, or for purification of the expressed antigen for use in a subunit vaccine.

Various methods are known in the art and can be used to inactivate the recombinant replication-competent VSV particles described herein, for use as killed vaccines. Such methods include but are not limited to inactivation by use of formalin, betapropiolactone, gamma irradiation, and psoralen plus ultraviolet light.

In certain aspects, the disclosure provides compositions (e.g., pharmaceutical compositions, immunogenic and/or antigenic compositions, vaccines) comprising the recombinant VSV particles described herein and a carrier and/or excipient. In certain embodiments, the VSV particles are replication-competent. In certain embodiments, the VSV particles are inactivated.

Administration of the recombinant VSV particles described herein can be used as a method of immunostimulation, to boost the host's immune system, enhancing cell-mediated and/or humoral immunity, and facilitating the clearance of infectious agents or symptoms of a disease or disorder in a subject infected with SARS-CoV-2 (e.g., having COVID-19). The present disclosure thus provides a method of immunizing an animal, or treating or preventing various diseases or disorders in an animal, comprising administering to the animal an effective immunizing dose of a vaccine of the present disclosure.

In certain aspects, the disclosure provides a method of treating or preventing a disease or disorder in a subject comprising administering to the subject an effective amount of the recombinant VSV particles described herein to induce an immune response (e.g., a protective immune response) against a foreign protein. In certain embodiments, the foreign protein is a coronavirus S glycoprotein, or a fragment or a derivative thereof. In a specific embodiment, the S glycoprotein is derived from SARS-CoV-2. In certain embodiments, the disclosure provides a method for the treatment or prevention of a disease or disorder in a subject infected with SARS-CoV-2. In certain embodiments, the disease or disorder is COVID-19.

In certain aspects, the disclosure provides a method of treating or preventing a disease or disorder in a subject comprising administering to the subject an effective amount of the recombinant VSV particles described herein to induce the formation of neutralizing antibodies against a foreign protein. In certain embodiments, the foreign protein is a coronavirus S glycoprotein, or a fragment or a derivative thereof. In a specific embodiment, the S glycoprotein is derived from SARS-CoV-2. In certain embodiments, the disclosure provides a method for the treatment or prevention of a disease or disorder in a subject infected with SARS-CoV-2. In certain embodiments, the disease or disorder is COVID-19.

In certain embodiments directed to therapeutics, the recombinant VSV particles of the disclosure are administered therapeutically, for the treatment of a disease or disorder in a subject infected with SARS-CoV-2. In certain embodiments, the disease or disorder is COVID-19. In certain aspects, the disclosure provides a method of treating a subject infected with SARS-CoV-2 comprising administering to the subject an amount of the recombinant VSV particles described herein in an effective amount to target the subject's cells harboring the SARS-CoV-2.

In certain embodiments directed to vaccines, the recombinant VSV particles described herein are administered prophylactically, to prevent/protect against a SARS-CoV-2 infection and/or infectious disease (e.g., having COVID-19).

The immunogenic and/or antigenic compositions and vaccines described herein may be multivalent or univalent. Multivalent vaccines are made from recombinant VSV particles described herein that direct the expression of more than one foreign protein, from the same or different recombinant VSV particles. The recombinant VSV particles described herein can be administered alone or in combination with other therapies (examples of anti-viral therapies, including but not limited to α-interferon and vidarabine phosphate). Other therapies can also include, but are not limited to, an anti-inflammatory agent, an antimalarial agent, and an antibody or antigen-binding fragment thereof that specifically binds coronavirus spike protein and/or TMPRSS2. In some embodiments, an antimalarial agent is chloroquine or hydroxychloroquine. In some embodiments, an anti-inflammatory agent is an antibody such as sarilumab, tocilizumab, or gimsilumab. In some embodiments, an antibody that specifically binds TMPRSS2 is H1H7017N, as described in International Patent Pub, No. WO/2019/147831, which is incorporated herein in its entirely for all purposes.

Many methods may be used to introduce the immunogenic and/or antigenic compositions and vaccines described herein, such as, but not limited to oral, intradermal, intramuscular, intraperitoneal, intravenous, infusions, subcutaneous, intranasal routes, and via scarification (scratching through the top layers of skin, e.g., using a bifurcated needle).

In certain embodiments, the delivery route is intramuscular (IM). The muscles have a plentiful supply of blood, which helps ensure that the body absorbs the medication quickly. The tissue in the muscles can also hold more medication than fatty tissue. In certain embodiments, intramuscular injection is followed by electroporation.

In certain embodiments, the delivery route is oral or mucosal (whether oral or intranasal). Oral and mucosal delivery can stimulate mucosal immune responses, which can play a role in protecting the lungs from aerosol exposure (see e.g., Qiu et. al., “Mucosal Immunization of Cynomolgus Macaques with the VSVAG/ZEBOVGP Vaccine Stimulates Strong Ebola GP-Specific Immune Responses” PLoS One 2009; 4(5):e5547). Oral and mucosal delivery can be more easily deployed in the event of a pandemic, outbreak of disease, or a bioterrorist attack, and because these routes can also be widely self-administered, they can reduce the requirement for trained personnel, especially in areas where the virus is endemic. Mucosal delivery can include, for example, sublingual, translingual, buccal, and intranasal delivery. These delivery routes avoid the use of needles, which may be more acceptable to patients.

In certain embodiments, the delivery route is oral. In certain embodiments, oral delivery may comprise application on a solid physiologically acceptable base, or in a physiologically acceptable dispersion. In certain embodiments, the immunogenic and/or antigenic or vaccine may be provided on a sugar cube, on a bread cube, in buffered saline, in a physiologically acceptable oil vehicle, or the like.

The subject to which the immunogenic and/or antigenic composition or vaccine is administered can be humans, non-human primates, veterinary animals (e.g., cats, dogs, cows, horses, sheep, pigs, goats, hamsters, etc.), and experimental animal models of diseases (e.g., mice, rats, ferrets, monkeys, etc.). In a preferred embodiment, the subject is a human.

The immunogenic and/or antigenic compositions and vaccines described herein comprise an effective immunizing amount of one or more recombinant VSV particles described herein (live or inactivated, as the case may be) and a pharmaceutically acceptable carrier or excipient. Pharmaceutically acceptable carriers are well known in the art and include, but are not limited to, saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration, which is readily determined by one of skill in the art.

In certain embodiments, the immunogenic and/or antigenic composition or vaccine can also comprise minor amounts of wetting or emulsifying agents, or pH buffering agents. The immunogenic and/or antigenic composition or vaccine can be a liquid solution, suspension, emulsion, tablet, pill, capsule, sustained release formulation, or powder. Oral formulations can include one or more standard carriers such as pharmaceutical grades of mannitol, lactose, starch, gelatin, magnesium stearate, sodium saccharine, cellulose, magnesium carbonate, methylcellulose (e.g., 4000 cP, 25 cP, METHOCEL™ E3, E5, E6, E15, E50, E4M, E10M, F4, F5, F4M, K3, K100, K4M, K15M, K100M, K4M CR, K15M CR, K100M CR, E4M CR, E10M CR, K4M Premium, K15M Premium, K100M Premium, E4M Premium, E10M Premium, K4M Premium CR, K15M Premium CR, K100M Premium CR, E4M Premium CR, E10M Premium CR, and K100 Premium LV), monosodium glutamate, human serum albumin, fetal bovine serum, trehalose, alginate (e.g., BioReagent), guar gum, MUCOLOX™, etc. In certain embodiments, the formulation has an appropriate viscosity to maintain stability of the virus particles. In certain embodiments, the formulation has an appropriate carrier to allow the viral particles to maintain contact with mucosal membranes for an appropriate amount of time for them to be taken up.

The ingredients are supplied either separately or mixed together in unit dosage form, for example, as a dry lyophilized powder or water free concentrate in a hermetically sealed container such as an ampoule or sachette indicating the quantity of active agent. In certain embodiments where in the immunogenic and/or antigenic composition or vaccine is administered by injection, an ampoule of sterile diluent can be provided so that the ingredients may be mixed prior to administration.

In certain embodiments, lyophilized recombinant VSV particles described herein are provided in a first container and a second container comprises diluent (e.g., an aqueous solution of 50% glycerin, 0.25% phenol, and an antiseptic (e.g., 0.005% brilliant green)).

The precise dose of virus, or subunit vaccine, to be employed in the immunogenic and/or antigenic composition or vaccine will also depend on the route of administration, and the nature of the patient, and should be decided according to the judgment of the practitioner and each patient's circumstances according to standard clinical techniques. The immunogenic and/or antigenic composition or vaccine is administered in an amount sufficient to produce an immune response to the foreign protein in the host to which the recombinant VSV particle is administered.

In certain embodiments, the immunogenically and/or antigenically effective amount can comprise a dosage of about 10³ to about 10¹⁵ infectious units, about 10⁴ to about 10¹⁰ infectious units, about 10² to about 10⁶ infectious units, about 10³ to about 10⁵ infectious units, about 10⁵ to about 10⁹ infectious units, or about 10⁶ to about 10⁸ infectious units per dose is suitable, depending upon the age and species of the subject being treated, and the immunogen against which the immune response is desired. The dosage can be about 10, about 10², about 10³, about 10⁴, or about 10⁵ infectious units per dose to about 10⁴, about 10⁵, about 10⁶, about 10⁷, about 10⁸, about 10⁹, or about 10¹⁰ infectious units per dose. In certain embodiments, effective doses of the immunogenic and/or antigenic composition or vaccine described herein may also be extrapolated from dose-response curves derived from animal model test systems.

In certain embodiments, a boosting dose is used. In certain embodiments, the boosting dose can be any SARS-CoV-2 vaccine. In certain embodiments, the boosting dose comprises any of the recombinant VSV particle vaccines described herein. In certain embodiments, the boosting dose comprises the foreign protein or peptide in purified form, or a nucleic acid encoding the foreign protein or peptide, rather than using a recombinant VSV particle described herein. In certain embodiments, the boosting dose comprises the same SARS-COV-2 vaccine as the SARS-COV-2 vaccine it is boosting. In certain embodiments, the boosting dose comprises a SARS-COV-2 vaccine that is different than the SARS-COV-2 vaccine it is boosting.

In certain embodiments, the boosting dose comprises any of the recombinant VSV particle vaccines described herein. In certain embodiments, the boosting dose is used to boost any of the recombinant VSV particle vaccines described herein. In certain embodiments, the boosting dose is used to boost a SARS-CoV-2 vaccine other than the recombinant VSV particle vaccines described herein.

Many methods may be used to introduce the boosting dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the delivery route is intramuscular.

In certain embodiments, the boosting dose is administered after a single dose of the SARS-CoV-2 vaccine. In certain embodiments, boosting dose is administered after repeated doses of the SARS-CoV-2 vaccine (e.g., 2, 3, 4, or 5 doses). The period of time between SARS-COV-2 vaccine administration and the boosting dose can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. If more than one boost is performed, the subsequent boost can be administered 1 week, 2 weeks, 3 weeks, 4 weeks, 5 weeks, 6 weeks, 7 weeks, 8 weeks, 3 months, 4 months, 5 months, 6 months, or longer after the preceding boost. For example, the interval between any two boosts can be 4 weeks, 8 weeks, or 12 weeks. For example, the SARS-COV-2 vaccine may be administered twice (e.g., via injection) before the boosting dose is administered (e.g., orally) and the boost is repeated every 3 months.

In certain embodiments, a priming dose is used. In certain embodiments, the priming dose can be any SARS-CoV-2 vaccine. In certain embodiments, the priming dose comprises any of the recombinant VSV particle vaccines described herein. In certain embodiments, the priming dose comprises the foreign protein or peptide in purified form, or a nucleic acid encoding the foreign protein or peptide, rather than using a recombinant VSV particle described herein. In certain embodiments, the priming dose comprises the same SARS-COV-2 vaccine as the SARS-COV-2 vaccine it is priming. In certain embodiments, the priming dose comprises a SARS-COV-2 vaccine that is different than the SARS-COV-2 vaccine it is priming.

In certain embodiments, the priming dose comprises any of the recombinant VSV particle vaccines described herein. In certain embodiments, the priming dose is used to prime any of the recombinant VSV particle vaccines described herein. In certain embodiments, the priming dose is used to prime a SARS-CoV-2 vaccine other than any of the recombinant VSV particle vaccines described herein.

Many methods may be used to introduce the priming dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the priming dose is administered via intramuscular injection.

The period of time between the priming dose and the SARS-COV-2 vaccine administration can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. For example, the interval between the priming dose and the SARS-COV-2 vaccine can be 4 weeks, 8 weeks, or 12 weeks. For example, the priming dose may be administered (e.g., via injection) before the SARS-COV-2 vaccine is administered.

Non-limiting examples of SARS-CoV-2 vaccines other than the recombinant VSV particle vaccines described herein include AZD1222 (ChAdOx1 nCoV-19; AstraZeneca and University of Oxford), mRNA-1273 (Moderna), BNT162a1 (Pfizer and BioNTech), BNT162b1 (Pfizer and BioNTech), BNT162b2 (Pfizer and BioNTech), BNT162c2 (Pfizer and BioNTech), INO-4800 (Inovio), Ad5-nCoV (CanSino Biotechnology), BBIP-CorV (Sinopharm), CoronaVac (PiCoVacc; Sinovac), Ad26.COV2-S (Johnson & Johnson), NVX-CoV2373 (with or without Matrix M adjuvant; Novavax), Gam-COVID-Vac (Gamaleya Research Institute), CVnCoV (CureVac), COVAC1 (Imperial College London), GX-19 (Genexine), AG0301 (AnGes), ZyCoV-D (Zydus Cadila), BBV152 (Bharat Biotech), SCB-2019 (Clover Biopharmaceuticals), COVAX-19 (Vaxine), KPB-COVID-19 (Kentucky BioProcessing), UQ COVID-19 (University of Queensland and CSL), CoVLP (Medicago), or combinations thereof.

In certain aspects, the disclosure also provides a kit or pharmaceutical pack comprising one or more containers comprising one or more of the ingredients of the immunogenic and/or antigenic composition or vaccine described herein. Associated with such container(s) can optionally be instructions and/or a notice in the form prescribed by a governmental agency regulating the manufacture, use or sale of pharmaceuticals or biological products, which notice reflects approval by the agency of manufacture, use or sale for administration (e.g., human administration).

In certain aspects, the disclosure provides a vaccine formulation that increases the amount of time the virus particles remain viable at 4° C. In certain embodiments, the vaccine formulation increases the amount of time the virus particles remain viable at 4° C. to at least about one week, at least about ten days, at least about two weeks, at least about three weeks, at least about four weeks, at least about five weeks, at least about six weeks, at least about seven weeks, at least about eight weeks, at least about nine weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, at least about 12 months, or at least about 2 years. In certain embodiments, the vaccine formulation increases the amount of time the virus particles remain viable at 4° C. to at least about two weeks. For example, virus titers remain at about three times titer range from day 0 mean.

In another aspect, the disclosure provides a vaccine formulation that allows at least 3 freeze/thaw cycles of the virus particles while maintaining viability. In certain embodiments, the vaccine formulation allows for at least 3 freeze/thaw cycles of the virus particles while maintaining at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% viability. In certain embodiments, the vaccine formulations allow at least 3 freeze/thaw cycles of the virus particles while maintaining at least about 30% viability.

In another aspect, the disclosure provides a vaccine formulation that improves contact time of the viral particles with mucous membranes, especially within the mouth. In certain embodiments, the vaccine formulation allows the viral particles to remain viable while in contact with the mucous membranes, especially for the extended contact time.

Antibodies Generated by the Immunogenic and/or Antigenic Compositions and Vaccines

In one aspect, the disclosure provides a method for generating antibodies against the foreign protein using the recombinant VSV particles described herein. The generated antibodies may be isolated by standard techniques known in the art (e.g., immunoaffinity chromatography, centrifugation, precipitation, etc.).

Antibodies generated against the foreign protein by immunization with the recombinant VSV particles described herein also have potential uses in diagnostic immunoassays and passive immunotherapy.

Assays in which the antibodies generated by the recombinant VSV particles described herein can be used include, but are not limited to, competitive and noncompetitive assay systems using techniques such as radioimmunoassays, ELISA (enzyme-linked immunosorbent assays), “sandwich” immunoassays, precipitin reactions, gel diffusion precipitin reactions, immunodiffusion assays, agglutination assays, complement-fixation assays, immunoradiometric assays, fluorescent immunoassays, protein A immunoassays and immunoelectrophoresis assays, etc.

### Determining Vaccine Efficacy

In one aspect, the disclosure provides a method for determining the efficacy of the immunogenic and/or antigenic composition or vaccine by measuring for the presence of a coronavirus neutralizing antibody in a sample. To determine immunogenicity or antigenicity, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, plaque-reduction neutralization (e.g., as described in Ayala-Breton et al., Hum. Gene Ther., 23:484-491 (2012) and incorporated by reference herein in its entirety), gel diffusion precipitin reactions, immunodiffusion assays, in situ immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays

In one embodiment, the sample is contacted with, or incubated with a recombinant vesicular stomatitis virus (VSV) particle, where the VSV glycoprotein (G) is replaced by a coronavirus spike (S) glycoprotein or a fragment or derivative thereof, wherein said S glycoprotein, fragment or derivative is capable of mediating infection of susceptible target cells. Afterwards, the recombinant VSV particle is contacted with a first target cell expressing a first portion of a reporter protein and a second target cell expressing a second portion of the reporter protein to form a fused cell comprising both the first and the second portion of the reporter protein and producing a detectable reporter signal. The first target cell and the second target cell should be capable of fusing with one another if contacted with the recombinant VSV particle. The reporter signal is measured in the fused cells and compared with a control.

The first portion of the reporter protein may comprise amino acids 1-229 of Renilla luciferase or a mutant thereof and the second portion of the reporter protein may comprise amino acids 230-311 of Renilla luciferase or a mutant thereof. The first portion of the reporter protein may comprise amino acids 1-155 of Renilla luciferase or a mutant thereof and the second portion of the reporter protein may comprise amino acids 156-311 of Renilla luciferase or a mutant thereof. The first portion of the reporter protein may comprise amino acids 1-157 of green fluorescent protein (GFP), and the second portion of the reporter protein may comprise amino acids 158-238 of GFP. The first portion of the reporter protein may comprise amino acids 1-213 of superfolder GFP, and the second portion of the reporter protein may comprise amino acids 214-230 of superfolder GFP. The first portion of the reporter protein may comprise amino acids 1-154 of superfolder yellow fluorescent protein (YFP), and the second portion of the reporter protein may comprise amino acids 155-262 of superfolder YFP. In certain embodiments, the first cell is Vero-DSP-1-Puro (CLR-73) and the second cell is Vero-DSP-2-Puro (CLR-74). Vero-DSP-1-Puro and Vero-DSP-2-Puro are generated by lentivirus transduction of Vero cells. In a specific embodiment, the luciferase mutant is RLuc8 which comprises the mutations A55T, C124A, S130A, K136R, A143M, M185V, M253L, and S287L

In another aspect, the disclosure provides a method for determining the efficacy of the immunogenic and/or antigenic composition or vaccine by measuring for the presence of a coronavirus neutralizing antibody in a sample, wherein the sample is contacted with a recombinant vesicular stomatitis virus (VSV) particle where the VSV glycoprotein (G) is replaced by a coronavirus spike (S) glycoprotein or a fragment or a derivative thereof, wherein said S glycoprotein, fragment or derivative is capable of mediating infection of a target cell and wherein the VSV particle comprises a reporter protein or a nucleic acid molecule encoding the reporter protein. The recombinant VSV particle is then contacted with the target cell. The reporter signal is then measured and compared with a control. In certain embodiments, the reporter protein is encoded by the genome of the recombinant VSV particle. In certain embodiments, the reporter protein is incorporated into the recombinant VSV particle without being encoded by the genome of the viral particle. The nucleic acid sequence encoding the reporter protein may be inserted between the nucleic acid sequence encoding the S glycoprotein and the nucleic acid sequence encoding VSV L protein. The target cell may be a Vero cell or any other cell comprising an angiotensin-converting enzyme 2 (ACE2) and in some instances serine protease TMPRSS2.

The sample used in the above methods of the disclosure may be, e.g., serum or plasma (e.g., heat-inactivated serum or plasma). In certain embodiments, in the first step the sample is contacted with the recombinant VSV particle for about 1 hour at about 37° C. and in the second step the recombinant VSV particle with the target cell may be conducted for 1-12, 1-3, 2-4, 3-5, 4-6, 5-7, 6-8, 7-9, or 8-10 hours at about 37° C.

In various embodiments, the methods comprise adding the reporter protein substrate for obtaining the reporter signal. The reporter protein may be a luciferase and the reporter protein substrate may be Luciferin or EnduRen luciferase substrate.

### Sequences

Amino Acid Sequence of SARS-CoV-2
(SEQ ID NO: 1; variant 1 and variant 4; NCBI
Reference Sequence: YP 009724390.1; SI domain is underlined;
RBD site is shown in bold)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT
Codon Optimized Polynucleotide Sequence of SARS-CoV-2
(SEQ ID NO: 2: Variant 1 and variant 4)
atgttcgtcttcctggtcctgctgcctctggtctcctcacagtgcgtcaatctgacaactcg
gactcagctgccacctgcttatactaatagcttcaccagaggcgtgtactatcctgacaagg
tgtttagaagctccgtgctgcactctacacaggatctgtttctgccattctttagcaacgtg
acctggttccacgccatccacgtgagcggcaccaatggcacaaagcggttcgacaatcccgt
gctgccttttaacgatggcgtgtacttcgcctctaccgagaagagcaacatcatcagaggct
ggatctttggcaccacactggactccaagacacagtctctgctgatcgtgaacaatgccacc
aacgtggtcatcaaggtgtgcgagttccagttttgtaatgatcccttcctgggcgtgtacta
tcacaagaacaataagagctggatggagtccgagtttagagtgtattctagcgccaacaact
gcacatttgagtacgtgagccagcctttcctgatggacctggagggcaagcagggcaatttc
aagaacctgagggagttcgtgtttaagaatatcgacggctacttcaaaatctactctaagca
cacccccatcaacctggtgcgcgacctgcctcagggcttcagcgccctggagcccctggtgg
atctgcctatcggcatcaacatcacccggtttcagacactgctggccctgcacagaagctac
ctgacacccggcgactcctctagcggatggaccgccggcgctgccgcctactatgtgggcta
cctccagccccggaccttcctgctgaagtacaacgagaatggcaccatcacagacgcagtgg
attgcgccctggaccccctgagcgagacaaagtgtacactgaagtcctttaccgtggagaag
ggcatctatcagacatccaatttcagggtgcagccaaccgagtctatcgtgcgctttcctaa
tatcacaaacctgtgcccatttggcgaggtgttcaacgcaacccgcttcgccagcgtgtacg
cctggaataggaagcggatcagcaactgcgtggccgactatagcgtgctgtacaactccgcc
tctttcagcacctttaagtgctatggcgtgtcccccacaaagctgaatgacctgtgctttac
caacgtctacgccgattctttcgtgatcaggggcgacgaggtgcgccagatcgcccccggcc
agacaggcaagatcgcagactacaattataagctgccagacgatttcaccggctgcgtgatc
gcctggaacagcaacaatctggattccaaagtgggcggcaactacaattatctgtaccggct
gtttagaaagagcaatctgaagcccttcgagagggacatctctacagaaatctaccaggccg
gcagcaccccttgcaatggcgtggagggctttaactgttatttcccactccagtcctacggc
ttccagcccacaaacggcgtgggctatcagccttaccgcgtggtggtgctgagctttgagct
gctgcacgccccagcaacagtgtgcggccccaagaagtccaccaatctggtgaagaacaagt
gcgtgaacttcaacttcaacggcctgaccggcacaggcgtgctgaccgagtccaacaagaag
ttcctgccatttcagcagttcggcagggacatcgcagataccacagacgccgtgcgcgaccc
acagaccctggagatcctggacatcacaccctgctctttcggcggcgtgagcgtgatcacac
ccggcaccaatacaagcaaccaggtggccgtgctgtatcaggacgtgaattgtaccgaggtg
cccgtggctatccacgccgatcagctgaccccaacatggcgggtgtacagcaccggctccaa
cgtcttccagacaagagccggatgcctgatcggagcagagcacgtgaacaattcctatgagt
gcgacatcccaatcggcgccggcatctgtgcctcttaccagacccagacaaactctcccaga
agagcccggagcgtggcctcccagtctatcatcgcctataccatgtccctgggcgccgagaa
cagcgtggcctactctaacaatagcatcgccatcccaaccaacttcacaatctctgtgacca
cagagatcctgcccgtgtccatgaccaagacatctgtggactgcacaatgtatatctgtggc
gattctaccgagtgcagcaacctgctgctccagtacggcagcttttgtacccagctgaatag
agccctgacaggcatcgccgtggagcaggataagaacacacaggaggtgttcgcccaggtga
agcaaatctacaagaccccccctatcaaggactttggcggcttcaatttttcccagatcctg
cctgatccatccaagccttctaagcggagctttatcgaggacctgctgttcaacaaggtgac
cctggccgatgccggcttcatcaagcagtatggcgattgcctgggcgacatcgcagccaggg
acctgatctgcgcccagaagtttaatggcctgaccgtgctgccacccctgctgacagatgag
atgatcgcacagtacacaagcgccctgctggccggcaccatcacatccggatggaccttcgg
cgcaggagccgccctccagatcccctttgccatgcagatggcctataggttcaacggcatcg
gcgtgacccagaatgtgctgtacgagaaccagaagctgatcgccaatcagtttaactccgcc
atcggcaagatccaggacagcctgtcctctacagccagcgccctgggcaagctccaggatgt
ggtgaatcagaacgcccaggccctgaataccctggtgaagcagctgagcagcaacttcggcg
ccatctctagcgtgctgaatgacatcctgagccggctggacaaggtggaggcagaggtgcag
atcgaccggctgatcaccggccggctccagagcctccagacctatgtgacacagcagctgat
cagggccgccgagatcagggccagcgccaatctggcagcaaccaagatgtccgagtgcgtgc
tgggccagtctaagagagtggacttttgtggcaagggctatcacctgatgtccttccctcag
tctgccccacacggcgtggtgtttctgcacgtgacctacgtgcccgcccaggagaagaactt
caccacagcccctgccatctgccacgatggcaaggcccactttccaagggagggcgtgttcg
tgtccaacggcacccactggtttgtgacacagcgcaatttctacgagccccagatcatcacc
acagacaacaccttcgtgagcggcaactgtgacgtggtcatcggcatcgtgaacaataccgt
gtatgatccactccagcccgagctggacagctttaaggaggagctggataagtatttcaaga
atcacacctcccctgacgtggatctgggcgacatcagcggcatcaatgcctccgtggtgaac
atccagaaggagatcgaccgcctgaacgaggtggctaagaatctgaacgagagcctgatcga
cctccaggagctgggcaagtatgagcagtacatcaagtggccctggtacatctggctgggct
tcatcgccggcctgatcgccatcgtgatggtgaccatcatgctgtgctgtatgacatcctgc
tgttcttgcctgaagggctgctgtagctgtggctcctgctgtaagtttgacgaggatgactc
tgaacctgtgctgaagggcgtgaagctgcattacacctaa
Amino Acid Sequence of SARS-CoV-2 A19CT
(SEQ ID NO: 3; variant 2; SI domain is
underlined; RBD site is shown in bold)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCC
Codon Optimized Polynucleotide Sequence of SARS-CoV-2 A19CT
(SEQ ID NO: 4; variant2)
atgttcgtcttcctggtcctgctgcctctggtctcctcacagtgcgtcaatctgacaactcg
gactcagctgccacctgcttatactaatagcttcaccagaggcgtgtactatcctgacaagg
tgtttagaagctccgtgctgcactctacacaggatctgtttctgccattctttagcaacgtg
acctggttccacgccatccacgtgagcggcaccaatggcacaaagcggttcgacaatcccgt
gctgccttttaacgatggcgtgtacttcgcctctaccgagaagagcaacatcatcagaggct
ggatctttggcaccacactggactccaagacacagtctctgctgatcgtgaacaatgccacc
aacgtggtcatcaaggtgtgcgagttccagttttgtaatgatcccttcctgggcgtgtacta
tcacaagaacaataagagctggatggagtccgagtttagagtgtattctagcgccaacaact
gcacatttgagtacgtgagccagcctttcctgatggacctggagggcaagcagggcaatttc
aagaacctgagggagttcgtgtttaagaatatcgacggctacttcaaaatctactctaagca
cacccccatcaacctggtgcgcgacctgcctcagggcttcagcgccctggagcccctggtgg
atctgcctatcggcatcaacatcacccggtttcagacactgctggccctgcacagaagctac
ctgacacccggcgactcctctagcggatggaccgccggcgctgccgcctactatgtgggcta
cctccagccccggaccttcctgctgaagtacaacgagaatggcaccatcacagacgcagtgg
attgcgccctggaccccctgagcgagacaaagtgtacactgaagtcctttaccgtggagaag
ggcatctatcagacatccaatttcagggtgcagccaaccgagtctatcgtgcgctttcctaa
tatcacaaacctgtgcccatttggcgaggtgttcaacgcaacccgcttcgccagcgtgtacg
cctggaataggaagcggatcagcaactgcgtggccgactatagcgtgctgtacaactccgcc
tctttcagcacctttaagtgctatggcgtgtcccccacaaagctgaatgacctgtgctttac
caacgtctacgccgattctttcgtgatcaggggcgacgaggtgcgccagatcgcccccggcc
agacaggcaagatcgcagactacaattataagctgccagacgatttcaccggctgcgtgatc
gcctggaacagcaacaatctggattccaaagtgggcggcaactacaattatctgtaccggct
gtttagaaagagcaatctgaagcccttcgagagggacatctctacagaaatctaccaggccg
gcagcaccccttgcaatggcgtggagggctttaactgttatttcccactccagtcctacggc
ttccagcccacaaacggcgtgggctatcagccttaccgcgtggtggtgctgagctttgagct
gctgcacgccccagcaacagtgtgcggccccaagaagtccaccaatctggtgaagaacaagt
gcgtgaacttcaacttcaacggcctgaccggcacaggcgtgctgaccgagtccaacaagaag
ttcctgccatttcagcagttcggcagggacatcgcagataccacagacgccgtgcgcgaccc
acagaccctggagatcctggacatcacaccctgctctttcggcggcgtgagcgtgatcacac
ccggcaccaatacaagcaaccaggtggccgtgctgtatcaggacgtgaattgtaccgaggtg
cccgtggctatccacgccgatcagctgaccccaacatggcgggtgtacagcaccggctccaa
cgtcttccagacaagagccggatgcctgatcggagcagagcacgtgaacaattcctatgagt
gcgacatcccaatcggcgccggcatctgtgcctcttaccagacccagacaaactctcccaga
agagcccggagcgtggcctcccagtctatcatcgcctataccatgtccctgggcgccgagaa
cagcgtggcctactctaacaatagcatcgccatcccaaccaacttcacaatctctgtgacca
cagagatcctgcccgtgtccatgaccaagacatctgtggactgcacaatgtatatctgtggc
gattctaccgagtgcagcaacctgctgctccagtacggcagcttttgtacccagctgaatag
agccctgacaggcatcgccgtggagcaggataagaacacacaggaggtgttcgcccaggtga
agcaaatctacaagaccccccctatcaaggactttggcggcttcaatttttcccagatcctg
cctgatccatccaagccttctaagcggagctttatcgaggacctgctgttcaacaaggtgac
cctggccgatgccggcttcatcaagcagtatggcgattgcctgggcgacatcgcagccaggg
acctgatctgcgcccagaagtttaatggcctgaccgtgctgccacccctgctgacagatgag
atgatcgcacagtacacaagcgccctgctggccggcaccatcacatccggatggaccttcgg
cgcaggagccgccctccagatcccctttgccatgcagatggcctataggttcaacggcatcg
gcgtgacccagaatgtgctgtacgagaaccagaagctgatcgccaatcagtttaactccgcc
atcggcaagatccaggacagcctgtcctctacagccagcgccctgggcaagctccaggatgt
ggtgaatcagaacgcccaggccctgaataccctggtgaagcagctgagcagcaacttcggcg
ccatctctagcgtgctgaatgacatcctgagccggctggacaaggtggaggcagaggtgcag
atcgaccggctgatcaccggccggctccagagcctccagacctatgtgacacagcagctgat
cagggccgccgagatcagggccagcgccaatctggcagcaaccaagatgtccgagtgcgtgc
tgggccagtctaagagagtggacttttgtggcaagggctatcacctgatgtccttccctcag
tctgccccacacggcgtggtgtttctgcacgtgacctacgtgcccgcccaggagaagaactt
caccacagcccctgccatctgccacgatggcaaggcccactttccaagggagggcgtgttcg
tgtccaacggcacccactggtttgtgacacagcgcaatttctacgagccccagatcatcacc
acagacaacaccttcgtgagcggcaactgtgacgtggtcatcggcatcgtgaacaataccgt
gtatgatccactccagcccgagctggacagctttaaggaggagctggataagtatttcaaga
atcacacctcccctgacgtggatctgggcgacatcagcggcatcaatgcctccgtggtgaac
atccagaaggagatcgaccgcctgaacgaggtggctaagaatctgaacgagagcctgatcga
cctccaggagctgggcaagtatgagcagtacatcaagtggccctggtacatctggctgggct
tcatcgccggcctgatcgccatcgtgatggtgaccatcatgctgtgctgtatgacatcctgc
tgttcttgcctgaagggctgctgtagctgtggctcctgctgttaa
Amino Acid Sequence of SARS-CoV-2 VSV-G CT
(SEQ ID NO: 5; variant 3; the sequence of VSV G
cvtoplasmic tail is shown in bold, underline)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMKLKHTKKRQIYTDIEMNRLGK
Codon Optimized Polynucleotide Sequence of SARS-CoV-2
VSV-G CT (SEQ ID NO: 6: variant 3;
the sequence encoding VSV G
cytoplasmic tail is shown in CAPs)
atgttcgtcttcctggtcctgctgcctctggtctcctcacagtgcgtcaatctgacaactcg
gactcagctgccacctgcttatactaatagcttcaccagaggcgtgtactatcctgacaagg
tgtttagaagctccgtgctgcactctacacaggatctgtttctgccattctttagcaacgtg
acctggttccacgccatccacgtgagcggcaccaatggcacaaagcggttcgacaatcccgt
gctgccttttaacgatggcgtgtacttcgcctctaccgagaagagcaacatcatcagaggct
ggatctttggcaccacactggactccaagacacagtctctgctgatcgtgaacaatgccacc
aacgtggtcatcaaggtgtgcgagttccagttttgtaatgatcccttcctgggcgtgtacta
tcacaagaacaataagagctggatggagtccgagtttagagtgtattctagcgccaacaact
gcacatttgagtacgtgagccagcctttcctgatggacctggagggcaagcagggcaatttc
aagaacctgagggagttcgtgtttaagaatatcgacggctacttcaaaatctactctaagca
cacccccatcaacctggtgcgcgacctgcctcagggcttcagcgccctggagcccctggtgg
atctgcctatcggcatcaacatcacccggtttcagacactgctggccctgcacagaagctac
ctgacacccggcgactcctctagcggatggaccgccggcgctgccgcctactatgtgggcta
cctccagccccggaccttcctgctgaagtacaacgagaatggcaccatcacagacgcagtgg
attgcgccctggaccccctgagcgagacaaagtgtacactgaagtcctttaccgtggagaag
ggcatctatcagacatccaatttcagggtgcagccaaccgagtctatcgtgcgctttcctaa
tatcacaaacctgtgcccatttggcgaggtgttcaacgcaacccgcttcgccagcgtgtacg
cctggaataggaagcggatcagcaactgcgtggccgactatagcgtgctgtacaactccgcc
tctttcagcacctttaagtgctatggcgtgtcccccacaaagctgaatgacctgtgctttac
caacgtctacgccgattctttcgtgatcaggggcgacgaggtgcgccagatcgcccccggcc
agacaggcaagatcgcagactacaattataagctgccagacgatttcaccggctgcgtgatc
gcctggaacagcaacaatctggattccaaagtgggcggcaactacaattatctgtaccggct
gtttagaaagagcaatctgaagcccttcgagagggacatctctacagaaatctaccaggccg
gcagcaccccttgcaatggcgtggagggctttaactgttatttcccactccagtcctacggc
ttccagcccacaaacggcgtgggctatcagccttaccgcgtggtggtgctgagctttgagct
gctgcacgccccagcaacagtgtgcggccccaagaagtccaccaatctggtgaagaacaagt
gcgtgaacttcaacttcaacggcctgaccggcacaggcgtgctgaccgagtccaacaagaag
ttcctgccatttcagcagttcggcagggacatcgcagataccacagacgccgtgcgcgaccc
acagaccctggagatcctggacatcacaccctgctctttcggcggcgtgagcgtgatcacac
ccggcaccaatacaagcaaccaggtggccgtgctgtatcaggacgtgaattgtaccgaggtg
cccgtggctatccacgccgatcagctgaccccaacatggcgggtgtacagcaccggctccaa
cgtcttccagacaagagccggatgcctgatcggagcagagcacgtgaacaattcctatgagt
gcgacatcccaatcggcgccggcatctgtgcctcttaccagacccagacaaactctcccaga
agagcccggagcgtggcctcccagtctatcatcgcctataccatgtccctgggcgccgagaa
cagcgtggcctactctaacaatagcatcgccatcccaaccaacttcacaatctctgtgacca
cagagatcctgcccgtgtccatgaccaagacatctgtggactgcacaatgtatatctgtggc
gattctaccgagtgcagcaacctgctgctccagtacggcagcttttgtacccagctgaatag
agccctgacaggcatcgccgtggagcaggataagaacacacaggaggtgttcgcccaggtga
agcaaatctacaagaccccccctatcaaggactttggcggcttcaatttttcccagatcctg
cctgatccatccaagccttctaagcggagctttatcgaggacctgctgttcaacaaggtgac
cctggccgatgccggcttcatcaagcagtatggcgattgcctgggcgacatcgcagccaggg
acctgatctgcgcccagaagtttaatggcctgaccgtgctgccacccctgctgacagatgag
atgatcgcacagtacacaagcgccctgctggccggcaccatcacatccggatggaccttcgg
cgcaggagccgccctccagatcccctttgccatgcagatggcctataggttcaacggcatcg
gcgtgacccagaatgtgctgtacgagaaccagaagctgatcgccaatcagtttaactccgcc
atcggcaagatccaggacagcctgtcctctacagccagcgccctgggcaagctccaggatgt
ggtgaatcagaacgcccaggccctgaataccctggtgaagcagctgagcagcaacttcggcg
ccatctctagcgtgctgaatgacatcctgagccggctggacaaggtggaggcagaggtgcag
atcgaccggctgatcaccggccggctccagagcctccagacctatgtgacacagcagctgat
cagggccgccgagatcagggccagcgccaatctggcagcaaccaagatgtccgagtgcgtgc
tgggccagtctaagagagtggacttttgtggcaagggctatcacctgatgtccttccctcag
tctgccccacacggcgtggtgtttctgcacgtgacctacgtgcccgcccaggagaagaactt
caccacagcccctgccatctgccacgatggcaaggcccactttccaagggagggcgtgttcg
tgtccaacggcacccactggtttgtgacacagcgcaatttctacgagccccagatcatcacc
acagacaacaccttcgtgagcggcaactgtgacgtggtcatcggcatcgtgaacaataccgt
gtatgatccactccagcccgagctggacagctttaaggaggagctggataagtatttcaaga
atcacacctcccctgacgtggatctgggcgacatcagcggcatcaatgcctccgtggtgaac
atccagaaggagatcgaccgcctgaacgaggtggctaagaatctgaacgagagcctgatcga
cctccaggagctgggcaagtatgagcagtacatcaagtggccctggtacatctggctgggct
tcatcgccggcctgatcgccatcgtgatggtgaccatcatgctgtgctgtatgAAATTAAAG
CACACCAAGAAAAGACAGATTTATACAGACATAGAGATGAACCGACTTGGAAAGTAA
Amino Acid Sequence of Mutant VSV Matrix
(M) Protein (M5 IR)
(SEQ ID NO: 7)
MSSLKKILGLKGKGKKSKKLGIAPPPYEEDTSMEYAPSAPIDKSYFGVDERDTHDPNQLRYE
KSFFTVKMTVRSNRPFRTYSDVAAAVSHWDHMYIGMAGKRPFYKILAFLGSSNLKATPAVLA
DQGQPEYHAHCEGRAYLPHRMGKTPPMLNVPEHFRRPFNIGLYKGTIELTMTIYDDESLEAA
PMIWDHFNSSKFSDFREKALMFGLIVEEEASGAWVLDSVRHSKWASLASSF
Polynucleotide Sequence of Mutant VSV Matrix
(M) Protein (M51R) (SEQ ID NO: 8)
ATGAGTTCCTTAAAGAAGATTCTCGGTCTGAAGGGGAAAGGTAAGAAATCTAAG
AAATTAGGGATCGCACCACCCCCTTATGAAGAGGACACTAGCATGGAGTATGCT
CCGAGCGCTCCAATTGACAAATCCTATTTTGGAGTTGACGAGCGAGACACCTATG
ATCCGAATCAATTAAGATATGAGAAATTCTTCTTTACAGTGAAAATGACGGTTAG
ATCTAATCGTCCGTTCAGAACATACTCAGATGTGGCAGCCGCTGTATCCCATTGG
GATCACATGTACATCGGAATGGCAGGGAAACGTCCCTTCTACAAAATCTTGGCTT
TTTTGGGTTCTTCTAATCTAAAGGCCACTCCAGCGGTATTGGCAGATCAAGGTCA
ACCAGAGTATCACGCTCACTGCGAAGGCAGGGCTTATTTGCCACATAGGATGGG
GAAGACCCCTCCCATGCTCAATGTACCAGAGCACTTCAGAAGACCATTCAATATA
GGTCTTTACAAGGGAACGATTGAGCTCACAATGACCATCTACGATGATGAGTCA
CTGGAAGCAGCTCCTATGATCTGGGATCATTTCAATTCTTCCAAATTTTCTGATTT
CAGAGAGAAGGCCTTAATGTTTGGCCTGATTGTCGAGAAAAAGGCATCTGGAGC
GTGGGTCCTGGACTCTATCGGCCACTTCAAATGA
Amino Acid Sequence of the Wild-Type VSV
Matrix (M) Protein (SEQ ID NO: 9)
MSSLKKILGLKGKGKKSKKLGIAPPPYEEDTSMEYAPSAPIDKSYFGVDEMDTHDPNQLRYE
KSFFTVKMTVRSNRPFRTYSDVAAAVSHWDHMYIGMAGKRPFYKILAFLGSSNLKATPAVLA
DQGQPEYHAHCEGRAYLPHRMGKTPPMLNVPEHFRRPFNIGLYKGTIELTMTIYDDESLEAA
PMIWDHFNSSKFSDFREKALMFGLIVEEEASGAWVLDSVRHSKWASLASSF
Polynucleotide Sequence of the Wild-Type VSV
Matrix (M) Protein (SEQ ID NO: 10)
ATGAGTTCCTTAAAGAAGATTCTCGGTCTGAAGGGGAAAGGTAAGAAATCTAAG
AAATTAGGGATCGCACCACCCCCTTATGAAGAGGACACTAGCATGGAGTATGCT
CCGAGCGCTCCAATTGACAAATCCTATTTTGGAGTTGACGAGATGGACACCTATG
ATCCGAATCAATTAAGATATGAGAAATTCTTCTTTACAGTGAAAATGACGGTTAG
ATCTAATCGTCCGTTCAGAACATACTCAGATGTGGCAGCCGCTGTATCCCATTGG
GATCACATGTACATCGGAATGGCAGGGAAACGTCCCTTCTACAAAATCTTGGCTT
TTTTGGGTTCTTCTAATCTAAAGGCCACTCCAGCGGTATTGGCAGATCAAGGTCA
ACCAGAGTATCACGCTCACTGCGAAGGCAGGGCTTATTTGCCACATAGGATGGG
GAAGACCCCTCCCATGCTCAATGTACCAGAGCACTTCAGAAGACCATTCAATATA
GGTCTTTACAAGGGAACGATTGAGCTCACAATGACCATCTACGATGATGAGTCA
CTGGAAGCAGCTCCTATGATCTGGGATCATTTCAATTCTTCCAAATTTTCTGATTT
CAGAGAGAAGGCCTTAATGTTTGGCCTGATTGTCGAGAAAAAGGCATCTGGAGC
GTGGGTCCTGGACTCTATCGGCCACTTCAAATGA
wild-type VSV Kozak sequence (SEQ ID NO: 11)
CACTATG
optimized Kozak sequence (SEQ ID NO: 12)
CACCATG
Amino Acid Sequence of SARS-CoV-1 Spike
(S) protein (SEP ID NO: 13)
MFIFLLFLTLTSGSDLDRCTTFDDVQAPNYTQHTSSMRGVYYPDEIFRSDTLYLTQDLFLPF
YSNVTGFHTINHTFGNPVIPFKDGIYFAATEKSNVVRGWVFGSTMNNKSQSVIIINNSTNVV
IRACNFELCDNPFFAVSKPMGTQTHTMIFDNAFNCTFEYISDAFSLDVSEKSGNFKHLREFV
FKNKDGFLYVYKGYQPIDVVRDLPSGFNTLKPIFKLPLGINITNFRAILTAFSPAQDIWGTS
AAAYFVGYLKPTTFMLKYDENGTITDAVDCSQNPLAELKCSVKSFEIDKGIYQTSNFRVVPS
GDVVRFPNITNLCPFGEVFNATKFPSVYAWERKKISNCVADYSVLYNSTFFSTFKCYGVSAT
KLNDLCFSNVYADSFVVKGDDVRQIAPGQTGVIADYNYKLPDDFMGCVLAWNTRNIDATSTG
NYNYKYRYLRHGKLRPFERDISNVPFSPDGKPCTPPALNCYWPLNDYGFYTTTGIGYQPYRV
VVLSFELLNAPATVCGPKLSTDLIKNQCVNFNFNGLTGTGVLTPSSKRFQPFQQFGRDVSDF
TDSVRDPKTSEILDISPCSFGGVSVITPGTNASSEVAVLYQDVNCTDVSTAIHADQLTPAWR
IYSTGNNVNFSISITTEVMPVSMAKTSVDCNMYICGDSTECANLLLQYGSFCTQLNRALSGI
AAEQDRNTREVFAQVKQMYKTPTLKYFGGFNFSQILPDPLKPTKRSFIEDLLFNKVTLADAG
FMKQYGECLGDINARDLICAQKFNGLTVLPPLLTDDMIAAYTAALVSGTATAGWTFGAGAAL
QIPFAMQMAYRFNGIGVTQNVLYENQKQIANQFNKAISQIQESLTTTSTALGKLQDVVNQNA
QALNTLVKQLSSNFGAISSVLNDILSRLDKVEAEVQIDRLITGRLQSLQTYVTQQLIRAAEI
RASANLAATKMSECVLGQSKRVDFCGKGYHLMSFPQAAPHGVVFLHVTYVPSQERNFTTAPA
ICHEGKAYFPREGVFVFNGTSWFITQRNFFSPQIITTDNTFVSGNCDVVIGIINNTVYDPLQ
PELDSFKEELDKYFKNHTSPDVDLGDISGINASVVNIQKEIDRLNEVAKNLNESLIDLQELG
KYEQYIKWPWYVWLGFIAGLIAIVMVTILLCCMTSCCSCLKGACSCGSCCKFDEDDSEPVLK
GVKLHYT

TABLE 4
SARS-Cov Domains

| | Residues | Residues | |
|---|---|---|---|
| SARS-CoV-1 | SARS-CoV-1 | SARS-CoV-2 | % |
| domains | (SEQ ID NO: 13) | (SEQ ID NO: 1) | identity |
| Full protein | 1-1255 | | 75.9 |
| Signal peptide | 1-13 | | 53.9 |
| Extracellular | 14-1195 | | |
| Transmembrane | 1196-1216 | | |
| Cytoplasmic | 1217-1255 | | 97.4 |
| S1 | 14-667 | 14-684 | 63.6 |
| S2 | 668-1255 | | 90 |
| S2′ | 798-1255 | | 93 |
| Cleavage site | 667-668 | | 100 |
| Cleavage site | 797-798 | | 100 |
| Receptor-binding | 306-527 | 319-541 | 73.1 |
| domain (RBD) | | | |
| Fusion peptide | 770-788 | | 83.3 |

| CLUSTAL O(1.2.4) multiple sequence alignment of spike (S) glycoprotein | |
|---|---|
| sequences from SARS-CoV-1 (SEQ ID NO: 13) and SARS-CoV-2 (SEQ ID NO: 1) | |
| SARS1 | MFIFLLFLTLTSGSDLDRCTTFDDVQAPNYTQHTSSMRGVYYPDEIFRSDTLYLTQDLFL 60 |
| SARS2 | MFVFLVLLPLVSSQCVNLTT--RTQLPPAY--TNSFTRGVYYPDKVFRSSVLHSTQDLFL 56 |
| | **:**::* *.. :: * * * .* *******::***..*: ****** |
| SARS1 | PFYSNVTGFHTIN-------HTFGNPVIPFKDGIYFAATEKSNVVRGWVFGSTMNNKSQS 113 |
| SARS2 | PFFSNVTWFHAIHVSGTNGTKRFDNPVLPFNDGVYFASTEKSNIIRGWIFGTTLDSKTQS 116 |
| | **:**** **:* * *.***:**:**:***:*****::***:**:*::.*:** |
| SARS1 | VIIINNSTNVVIRACNFELCDNPFFAVSKPMGTQTHTMIFDNAFNCTFEYISDAFS 169 |
| SARS2 | LLIVNNATNVVIKVCEFQFCNDPFLGVYYHKNNKSWMESEFRVYSSANNCTFEYVSQPFL 176 |
| | ::*:**:*****:.*:*::*::**:.* .. ::. ::..* ******:*: * |
| SARS1 | LDVSEKSGNFKHLREFVFKNKDGFLYVYKGYQPIDVVRDLPSGFNTLKPIFKLPLGINIT 229 |
| SARS2 | MDLEGKQGNFKNLREFVFKNIDGYFKIYSKHTPINLVRDLPQGFSALEPLVDLPIGINIT 236 |
| | :*:. *.****:******** **:: :*. : **::*****.**.:*:*:..**.***** |
| SARS1 | NFRAILTAFS------PAQDIWGTSAAAYFVGYLKPTTFMLKYDENGTITDAVDCSQNPL 283 |
| SARS2 | RFQTLLALHRSYLTPGDSSSGWTAGAAAYYVGYLQPRTFLLKYNENGTITDAVDCALDPL 296 |
| | .*:::*: . :.. * :.****:****:* **:***:***********: :** |
| SARS1 | AELKCSVKSFEIDKGIYQTSNFRVVPSGDVVRFPNITNLCPFGEVFNATKFPSVYAWERK 343 |
| SARS2 | SETKCTLKSFTVEKGIYQTSNFRVQPTESIVRFPNITNLCPFGEVFNATRFASVYAWNRK 356 |
| | :* **::*** ::*********** .*:*********************:* *****:** |
| SARS1 | KISNCVADYSVLYNSTFFSTFKCYGVSATKLNDLCFSNVYADSFVVKGDDVRQIAPGQTG 403 |
| SARS2 | RISNCVADYSVLYNSASFSTFKCYGVSPTKLNDLCFTNVYADSFVIRGDEVRQIAPGQTG 416 |
| | :**************: ********** ********:********::**:********** |
| SARS1 | VIADYNYKLPDDFMGCVLAWNTRNIDATSTGNYNYKYRYLRHGKLRPFERDISNVPFSPD 463 |
| SARS2 | KIADYNYKLPDDFTGCVIAWNSNNLDSKVGGNYNYLYRLFRKSNLKPFERDISTEIYQAG 476 |
| | ************ ***:***:.*:*. ****** ** :*:.:*:****** :. . |
| SARS1 | GKPCTP-PALNCYWPLNDYGFYTTTGIGYQPYRVVVLSFELLNAPATVCGPKLSTDLIKN 522 |
| SARS2 | STPCNGVEGFNCYFPLQSYGFQPTNGVGYQPYRVVVLSFELLHAPATVCGPKKSTNLVKN 536 |
| | ..**. .:***:**:.*** *.*:***************:********* **:*:** |
| SARS1 | QCVNFNFNGLTGTGVLTPSSKRFQPFQQFGRDVSDFTDSVRDPKTSEILDISPCSFGGVS 582 |
| SARS2 | KCVNFNFNGLTGTGVLTESNKKFLPFQQFGRDIADTTDAVRDPQTLEILDITPCSFGGVS 596 |
| | :**************** *.*:* ********::* **:****:* *****:******** |
| SARS1 | VITPGTNASSEVAVLYQDVNCTDVSTAIHADQLTPAWRIYSTGNNV-------------- 628 |
| SARS2 | VITPGTNTSNQVAVLYQDVNCTEVPVAIHADQLTPTWRVYSTGSNVFQTRAGCLIGAEHV 656 |
| | *******:*.:***********:* .*********:**:****.** |
| SARS1 | ----------------------------------------------------------- 628 |
| SARS2 | NNSYECDIPIGAGICASYQTQTNSPRRARSVASQSIIAYTMSLGAENSVAYSNNSIAIPT 716 |
| SARS1 | NFSISITTEVMPVSMAKTSVDCNMYICGDSTECANLLLQYGSFCTQLNRALSGIAAEQDR 688 |
| SARS2 | NFTISVTTEILPVSMTKTSVDCTMYICGDSTECSNLLLQYGSFCTQLNRALTGIAVEQDK 776 |
| | **:**:***::****:******.**********:*****************:***.***: |
| SARS1 | NTREVFAQVKQMYKTPTLKYFGGFNFSQILPDPLKPTKRSFIEDLLFNKVTLADAGFMKQ 748 |
| SARS2 | NTQEVFAQVKQIYKTPPIKDFGGFNFSQILPDPSKPSKRSFIEDLLFNKVTLADAGFIKQ 836 |
| | **:********:**** :* ************* **:********************:** |
| SARS1 | YGECLGDINARDLICAQKFNGLTVLPPLLTDDMIAAYTAALVSGTATAGWTFGAGAALQI 808 |
| SARS2 | YGDCLGDIAARDLICAQKFNGLTVLPPLLTDEMIAQYTSALLAGTITSGWTFGAGAALQI 896 |
| | **:***** **********************:*** **:**:: * *:************ |
| SARS1 | PFAMQMAYRFNGIGVTQNVLYENQKQIANQFNKAISQIQESLTTTSTALGKLQDVVNQNA 868 |
| SARS2 | PFAMQMAYRFNGIGVTQNVLYENQKLIANQFNSAIGKIQDSLSSTASALGKLQDVVNQNA 956 |
| | ************************* ******.**.:**:**::*::************* |
| SARS1 | QALNTLVKQLSSNFGAISSVLNDILSRLDKVEAEVQIDRLITGRLQSLQTYVTQQLIRAA 928 |
| SARS2 | QALNTLVKQLSSNFGAISSVLNDILSRLDKVEAEVQIDRLITGRLQSLQTYVTQQLIRAA 1016 |
| | ************************************************************ |
| SARS1 | EIRASANLAATKMSECVLGQSKRVDFCGKGYHLMSFPQAAPHGVVFLHVTYVPSQERNFT 988 |
| SARS2 | EIRASANLAATKMSECVLGQSKRVDFCGKGYHLMSFPQSAPHGVVFLHVTYVPAQEKNFT 1076 |
| | **************************************:**************:**:*** |
| SARS1 | TAPAICHEGKAYFPREGVFVFNGTSWFITQRNFFSPQIITTDNTFVSGNCDVVIGIINNT 1048 |
| SARS2 | TAPAICHDGKAHFPREGVFVSNGTHWFVTQRNFYEPQIITTDNTFVSGNCDVVIGIVNNT 1136 |
| | *******:***:******** *** **:*****:.*********************:*** |
| SARS1 | VYDPLQPELDSFKEELDKYFKNHTSPDVDLGDISGINASVVNIQKEIDRLNEVAKNLNES 1108 |
| SARS2 | VYDPLQPELDSFKEELDKYFKNHTSPDVDLGDISGINASVVNIQKEIDRLNEVAKNLNES 1196 |
| | ************************************************************ |
| SARS1 | LIDLQELGKYEQYIKWPWYVWLGFIAGLIAIVMVTILLCCMTSCCSCLKGACSCGSCCKF 1168 |
| SARS2 | LIDLQELGKYEQYIKWPWYIWLGFIAGLIAIVMVTIMLCCMTSCCSCLKGCCSCGSCCKF 1256 |
| | *******************:****************:*************.********* |
| SARS1 | DEDDSEPVLKGVKLHYT 1185 |
| SARS2 | DEDDSEPVLKGVKLHYT 1273 |
| | ***************** |

Amino Acid Sequence of SARS-CoV-2 Spike (S) protein
last 19 amino acids of the
cytoplasmic tail (SEQ ID NO: 14)
KFDEDDSEPVLKGVKLHYT
Amino Acid Sequence of VSV G cvtoplasmic tail (SEQ ID NO: 15)
KLKHTKKRQIYTDIEMNRLGK
Rluc8 155-156DSP1-7 luciferase-GFP fusion protein
(SEQ ID NO: 16; Renilla luciferase
fragment aa 1-155 is underlined; linker is not
highlighted: fragment aa 1-156 of engineered
GFP is shown in bold)
MASKVYDPEQRKRMITGPQWWARCKQMNVLDSFINYYDSEKHAENAVIFLHGNATSSYLWRH
VVPHIEPVARCIIPDLIGMGKSGKSGNGSYRLLDHYKYLTAWFELLNLPKKIIFVGHDWGAA
LAFHYAYEHQDRIKAIVHMESVVDVIESWDESGGGGMSKGEELFTGVVPILVELDGDVNGHK
FSVRGEGEGDATIGKLTLKFICTTGKLPVPWPTLVTTLTYGVQCFSRYPDHMKRHDFFKSAM
PEGYVQERTISFKDDGKYKTRAVVKFEGDTLVNRIELKGTDFKEDGNILGHKLEYNFNSHNV
YITADK
Rluc8 155-156DSP8-11 luciferase-GFP fusion
protein (SEQ ID NO: 17; Renilla luciferase
fragment aa 156-311 is underlined; linker
is not highlighted; fragment aa 157-231 of
engineered GFP is shown in bold)
MQKNGIKANFTVRHNVEDGSVQLADHYQQNTPIGDGPVLLPDNHYLSTQTVLSKDPNEKRDH
MVLHEYVNAAGITGGGGSWPDIEEDIALIKSEEGEKMVLENNFFVETVLPSKIMRKLEPEEF
AAYLEPFKEKGEVRRPTLSWPREIPLVKGGKPDVVQIVRNYNAYLRASDDLPKLFIESDPGF
FSNAIVEGAKKFPNTEFVKVKGLHFLQEDAPDEMGKYIKSFVERVLKNEQ
Engineered GFP (SEQ ID NO: 18)
MSKGEELFTGVVPILVELDGDVNGHKFSVRGEGEGDATIGKLTLKFICTTGKLPVPWPTLVT
TLTYGVQCFSRYPDHMKRHDFFKSAMPEGYVQERTISFKDDGKYKTRAVVKFEGDTLVNRIE
LKGTDFKEDGNILGHKLEYNFNSHNVYITADKMQKNGIKANFTVRHNVEDGSVQLADHYQQN
TPIGDGPVLLPDNHYLSTQTVLSKDPNEKRDHMVLHEYVNAAGIT
RLuc8 (SEQ ID NO: 19)
MASKVYDPEQRKRMITGPQWWARCKQMNVLDSFINYYDSEKHAENAVIFLHGNATSSYLWRH
VVPHIEPVARCIIPDLIGMGKSGKSGNGSYRLLDHYKYLTAWFELLNLPKKIIFVGHDWGAA
LAFHYAYEHQDRIKAIVHMESVVDVIESWDEWPDIEEDIALIKSEEGEKMVLENNFFVETVL
PSKIMRKLEPEEFAAYLEPFKEKGEVRRPTLSWPREIPLVKGGKPDVVQIVRNYNAYLRASD
DLPKLFIESDPGFFSNAIVEGAKKFPNTEFVKVKGLHFLQEDAPDEMGKYIKSFVERVLKNE
Q
Amino Acid Sequence of SARS-CoV-2 A19CT
CPE Lvtic Variant (SEQ ID NO: 20; CPE
Variant)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRRY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQNVNCTEV
PVAIHADQLTPTWRVYSTGSNVFQTRAGCLIGAEHVNNSYECDIPIGAGICASYQTQTNSPR
RAQSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCC
Codon Optimized Polynucleotide Sequence of
SARS-CoV-2 A19CT CPE Lvtic Variant
(SEQ ID NO: 21; CPE Variant)
atgttcgtcttcctggtcctgctgcctctggtctcctcacagtgcgtcaatctgacaactcg
gactcagctgccacctgcttatactaatagcttcaccagaggcgtgtactatcctgacaagg
tgtttagaagctccgtgctgcactctacacaggatctgtttctgccattctttagcaacgtg
acctggttccacgccatccacgtgagcggcaccaatggcacaaagcggttcgacaatcccgt
gctgccttttaacgatggcgtgtacttcgcctctaccgagaagagcaacattatcagaggct
ggatctttggcaccacactggactccaagacacagtctctgctgatcgtgaacaatgccacc
aacgtggtcatcaaggtgtgcgagttccagttttgtaatgatcccttcctgggcgtgtacta
tcacaagaacaataagagctggatggagtccgagtttagagtgtattctagcgccaacaact
gcacatttgagtacgtgagccagcctttcctgatggacctggagggcaagcagggcaatttc
aagaacctgagggagttcgtgtttaagaatatcgacggctacttcaaaatctactctaagca
cacccccatcaacctggtgcgcgacctgcctcagggcttcagcgccctggagcccctggtgg
atctgcctatcggcatcaacatcacccggtttcagacactgctggccctgcacagaagatac
ctgacacccggcgactcctctagcggatggaccgccggcgctgccgcctactatgtgggcta
cctccagccccggaccttcctgctgaagtacaacgagaatggcaccatcacagacgcagtgg
attgcgccctggaccccctgagcgagacaaagtgtacactgaagtcctttaccgtggagaag
ggcatctatcagacatccaatttcagggtgcagccaaccgagtctatcgtgcgctttcctaa
tatcacaaacctgtgcccatttggcgaggtgttcaacgcaacccgcttcgccagcgtgtacg
cctggaataggaagcggatcagcaactgcgtggccgactatagcgtgctgtacaactccgcc
tctttcagcacctttaagtgctatggcgtgtcccccacaaagctgaatgacctgtgctttac
caacgtctacgccgattctttcgtgatcaggggcgacgaggtgcgccagatcgcccccggcc
agacaggcaagatcgcagactacaattataagctgccagacgatttcaccggctgcgtgatc
gcctggaacagcaacaatctggattccaaagtgggcggcaactacaattatctgtaccggct
gtttagaaagagcaatctgaagcccttcgagagggacatctctacagaaatctaccaggccg
gcagcaccccttgcaatggcgtggagggctttaactgttatttcccactccagtcctacggc
ttccagcccacaaacggcgtgggctatcagccttaccgcgtggtggtgctgagctttgagct
gctgcacgccccagcaacagtgtgcggccccaagaagtccaccaatctggtgaagaacaagt
gcgtgaacttcaacttcaacggcctgaccggcacaggcgtgctgaccgagtccaacaagaag
ttcctgccatttcagcagttcggcagggacatcgcagataccacagacgccgtgcgcgaccc
acagaccctggagatcctggacatcacaccctgctctttcggcggcgtgagcgtgatcacac
ccggcaccaatacaagcaaccaggtggccgtgctgtatcagaacgtgaattgtaccgaggtg
cccgtggctatccacgccgatcagctgaccccaacatggcgggtgtacagcaccggctccaa
cgtcttccagacaagagccggatgcctgatcggagcagagcacgtgaacaattcctatgagt
gcgacatcccaatcggcgccggcatctgtgcctcttaccagacccagacaaactctcccaga
agagcccagagcgtggcctcccagtctatcatcgcctataccatgtccctgggcgccgagaa
cagcgtggcctactctaacaatagcatcgccatcccaaccaacttcacaatctctgtgacca
cagagatcctgcccgtgtccatgaccaagacatctgtggactgcacaatgtatatctgtggc
gattctaccgagtgcagcaacctgctgctccagtacggcagcttttgtacccagctgaatag
agccctgacaggcatcgccgtggagcaggataagaacacacaggaggtgttcgcccaggtga
agcaaatctacaagaccccccctatcaaggactttggcggcttcaatttttcccagatcctg
cctgatccatccaagccttctaagcggagctttatcgaggacctgctgttcaacaaggtgac
cctggccgatgccggcttcatcaagcagtatggcgattgcctgggcgacatcgcagccaggg
acctgatctgcgcccagaagtttaatggcctgaccgtgctgccacccctgctgacagatgag
atgatcgcacagtacacaagcgccctgctggccggcaccatcacatccggatggaccttcgg
cgcaggagccgccctccagatcccctttgccatgcagatggcctataggttcaacggcatcg
gcgtgacccagaatgtgctgtacgagaaccagaagctgatcgccaatcagtttaactccgcc
atcggcaagatccaggacagcctgtcctctacagccagcgccctgggcaagctccaggatgt
ggtgaatcagaacgcccaggccctgaataccctggtgaagcagctgagcagcaacttcggcg
ccatctctagcgtgctgaatgacatcctgagccggctggacaaggtggaggcagaggtgcag
atcgaccggctgatcaccggccggctccagagcctccagacctatgtgacacagcagctgat
cagggccgccgagatcagggccagcgccaatctggcagcaaccaagatgtccgagtgcgtgc
tgggccagtctaagagagtggacttttgtggcaagggctatcacctgatgtccttccctcag
tctgccccacacggcgtggtgtttctgcacgtgacctacgtgcccgcccaggagaagaactt
caccacagcccctgccatctgccacgatggcaaggcccactttccaagggagggcgtgttcg
tgtccaacggcacccactggtttgtgacacagcgcaatttctacgagccccagatcatcacc
acagacaacaccttcgtgagcggcaactgtgacgtggtcatcggcatcgtgaacaataccgt
gtatgatccactccagcccgagctggacagctttaaggaggagctggataagtatttcaaga
atcacacctcccctgacgtggatctgggcgacatcagcggcatcaatgcctccgtggtgaac
atccagaaggagatcgaccgcctgaacgaggtggctaagaatctgaacgagagcctgatcga
cctccaggagctgggcaagtatgagcagtacatcaagtggccctggtacatctggctgggct
tcatcgccggcctgatcgccatcgtgatggtgaccatcatgctgtgctgtatgacatcctgc
tgttcttgcctgaagggctgctgtagctgtggctcctgctgttaa
Amino Acid Sequence of SARS-CoV-2 A19CT
Variant (SEQ ID NO: 22; Variant)
MFVFLVLLPLVSSQCVNLTTRTQLPPAYTNSFTRGVYYPDKVFRSSVLHSTQDLFLPFFSNV
TWFHAISGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFKN
LREFVFKNIDGYFKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETKCTLKSFTVEKGI
YQTSNFRVQPTESIVRFPNITNLCPFGEVFNATRFASVYAWNRKRISNCVADYSVLYNSASF
STFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVKGFNCYFPLQSYGFQ
PTYGVGYQPYRVVVLSFELLHAPATVCGPKKSTNLVKNKCVNFNENGLTGTGVLTESNKKEL
PFQQFGRDIADTTDAVRDPQTLEILDITPCSFGGVSVITPGTNTSNQVAVLYQGVNCTEVPV
AIHADQLTPTWRVYSTGSNVFQTRAGCLIGAEHVNNSYECDIPIGAGICASYQTQTNSPRRA
RSVASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQILPD
PSKPSKRSFIEDLLFNKVTLADAGFIKQYGDCLGDIAARDLICAQKFNGLTVLPPLLTDEMI
AQYTSALLAGTITSGWTFGAGAALQIPFAMQMAYRFNGIGVTQNVLYENQKLIANQFNSAIG
KIQDSLSSTASALGKLQDVVNQNAQALNTLVKQLSSNFGAISSVLNDILSRLDKVEAEVQID
RLITGRLQSLQTYVTQQLIRAAEIRASANLAATKMSECVLGQSKRVDFCGKGYHLMSFPQSA
PHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTSCCS
CLKGCCSCGSCC

### EXAMPLES

The present disclosure is also described and demonstrated by way of the following examples. However, the use of this and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1: Preparation of Recombinant VSV Particles Expressing Spike (S)

### Glycoprotein and a Fragment and Derivative Thereof

Infectious clones of Indiana strain VSV were used to generate four recombinant VSV constructs, wherein the VSV (G) glycoprotein was deleted and replaced by codon optimized sequences suitable for expression in human cells and encoding: the full length SARS-CoV-2 spike (S) glycoprotein sequence (NCBI Reference Sequence: NC_045512.2; Protein_ID: YP 009724390.1;) (variant 1; VSV SARS-CoV-2 dG; amino acid sequence SEQ ID NO: 1; codon optimized coding polynucleotide sequence SEQ ID NO: 2); the SARS-CoV-2 S glycoprotein sequence with a deletion of the 19 amino acids KFDEDDSEPVLKGVKLHYT (SEQ ID NO: 14) at the C terminus (variant 2; VSV SARS-CoV-2 Δ19CT dG; amino acid sequence SEQ ID NO: 3; codon optimized coding polynucleotide sequence SEQ ID NO: 4); the SARS-CoV-2 S glycoprotein sequence with a replacement of the S glycoprotein cytoplasmic tail with VSV G cytoplasmic tail KLKHTKKRQIYTDIEMNRLGK (SEQ ID NO: 15) (variant 3; VSV SARS-CoV-2 VSV-G CT dG; amino acid sequence SEQ ID NO: 5; codon optimized coding polynucleotide sequence SEQ ID NO: 6); or variant 4—the full length SARS-CoV-2 S glycoprotein sequence (VSV SARS-CoV-2 dG; amino acid sequence SEQ ID NO: 1; codon optimized coding polynucleotide sequence SEQ ID NO: 2), with the wild-type VSV Kozak sequence (cActATG; SEQ ID NO: 11) in place of the optimized Kozak sequence (caccATG; SEQ ID NO: 12) used in the other three constructs. One set of variant 1-4 constructs (constructs 1-4) was prepared that encoded wild-type VSV M protein (amino acid sequence SEQ ID NO: 9; polynucleotide sequence SEQ ID NO: 10). A second set of variant 1-4 constructs (constructs 5-8) was prepared that encoded M protein with the substitution M51R (amino acid sequence SEQ ID NO: 7; polynucleotide sequence SEQ ID NO: 8), which results in virus attenuation. See FIG. 1.

The variant 1-4 recombinant viral particles were produced using a standard published protocol using transfection with vaccinia-T7 virus (expressing T7 polymerase) followed by co-transfection with N, P and L expression plasmids (with respective genes under the control of T7 promoter) and the viral genome plasmid. A plasmid expressing VSV G was also transfected into the cells to facilitate rescue. The viruses were amplified and propagated in Vero cells. The amplified recombinant viruses do not have VSV (G) glycoprotein and depend on SARS-CoV-2 spike (S) glycoprotein for entry and infection.

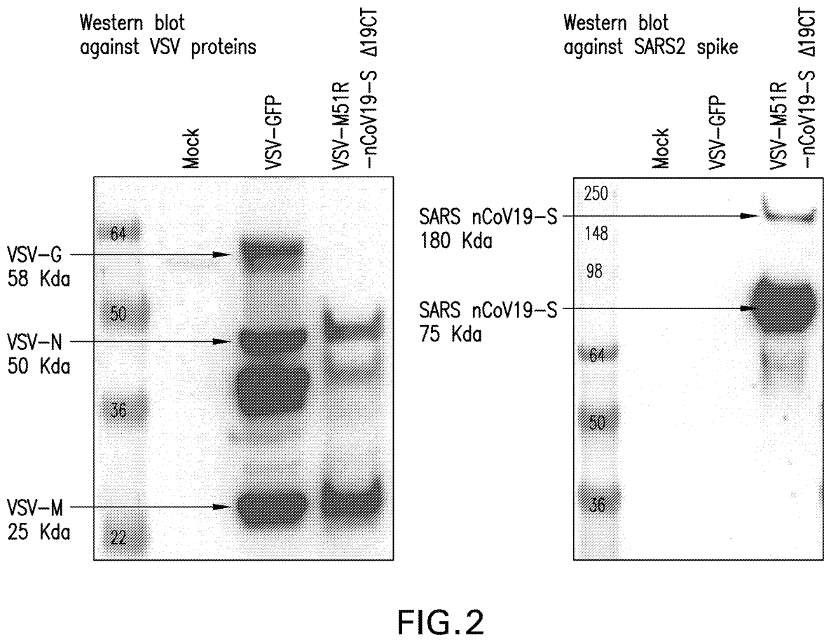

Correct incorporation of VSV G, N and M proteins and SARS-CoV-2 S glycoprotein in the recombinant variant 2 VSV SARS-CoV-2 Δ19CT dG construct 6 (VSV-M51R-nCoV19-S Δ19CT) virions was analyzed by Western blotting. The results are shown in FIG. 2. SARS-CoV-2 Δ19CT S glycoprotein produced two bands corresponding to the full-length (180 kDa) and the proteolytically cleaved (75 kDa) glycoprotein. The Western blot shows the presence of VSV N, M and G proteins in the parental VSV-GFP virus and the presence of VSV N and M proteins (but not VSV G glycoprotein) in the variant 2 VSV SARS-CoV-2 Δ19CT dG construct 6 (VSV-M51R-nCoV19-S Δ19CT) virus. The Western blot for variant 2 VSV SARS-CoV-2 Δ19CT dG construct 6 (VSV-M51R-nCoV19-S Δ19CT) virus also shows efficient incorporation of SARS-CoV-2 Δ19CT S glycoprotein in place of the VSV G glycoprotein.

### Example 2: Fusogenicity Assays of Recombinant VSV Particles Expressing S Glycoprotein Variant 1 and Variant 2 Demonstrate the Ability of the Recombinant VSV Particles to Infect Host Cells

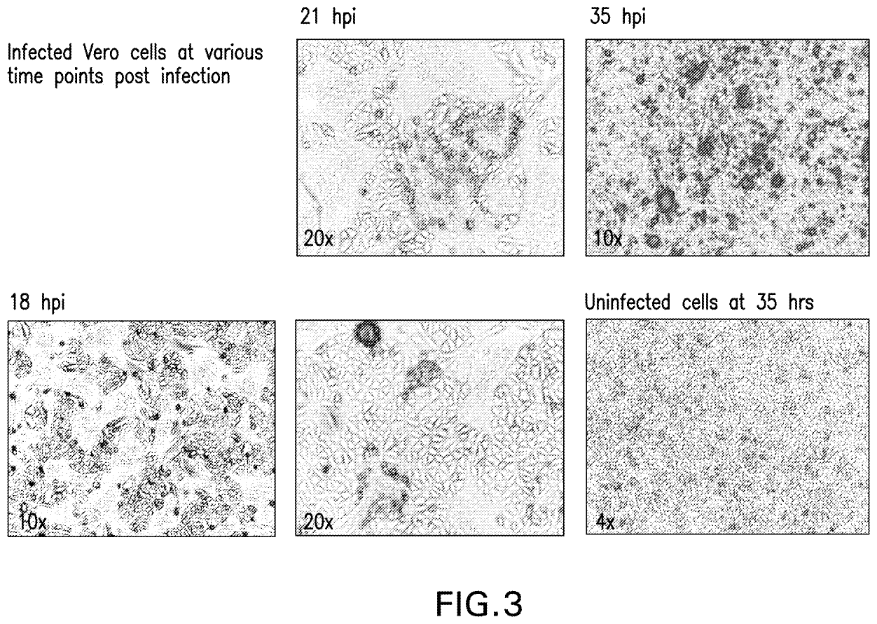

Recombinant variant 2 VSV SARS-CoV-2 Δ19CT dG construct 6 (VSV-M51R-nCoV19-S Δ19CT) viral particles were prepared as described above and were tested for fusogenicity by infecting Vero-αHis cells followed by microscopic observations. FIG. 3 depicts cells 18, 21, and 35 hours after being infected (hours post infection; hpi) showing that the recombinant variant 2 VSV SARS-CoV-2 Δ19CT dG construct 6 (VSV-M51R-nCoV19-S Δ19CT) viral particles successfully induced cell fusion.

It was also tested whether SARS-CoV-2 S glycoprotein-mediated cell fusion could be detected using luciferase signal resulting from virus-induced fusion of Vero-DSP1-Puro and Vero-DSP2-Puro cells. Vero-DSP-1-Puro (CLR-73) and Vero-DSP-2-Puro (CLR-74) cells are engineered Vero cells (African green monkey-derived kidney epithelial cells) that have been stably transduced by lentiviral vector transduction and puromycin selection to contain the dual split protein (DSP) reporter DSP1 or DSP2. Vero-DSP1-Puro cells express Rluc8 155-156DSP1-7 luciferase-GFP fusion protein (SEQ ID NO: 16) comprising RLuc8 mutant Renilla luciferase fragment amino acids 1-155 and engineered GFP fragment amino acids 1-156. Vero-DSP2-Puro cells express Rluc8 155-156DSP8-11 luciferase-GFP fusion protein (SEQ ID NO: 17) comprising RLuc8 mutant Renilla luciferase fragment amino acids 157-311 and engineered GFP fragment amino acids 157-231. RLuc8 mutant Renilla luciferase contains the mutations A55T, C124A, S130A, K136R, A143M, M185V, M253L, and S287L (see SEQ ID NO: 19). The sequence of engineered GFP is provided in SEQ ID NO: 18.

A Vero-DSP1-Puro/Vero-DSP2-Puro cell mixture was infected with variant 2 VSV SARS-CoV-2 Δ19CT dG construct 6 (VSV-M51R-nCOV2019-Δ19-dG), rinsed with OptiMem 4 hours after infection, and then treated with 4 μg/mL of trypsin in OptiMem. A control Vero-DSP1-PuroNero-DSP2-Puro cell mixture was infected with the same construct, but not treated with trypsin. Another control Vero-DSP1-PuroNero-DSP2-Puro cell mixture was not infected with the construct (mock) and was either treated with 4 μg/mL of trypsin in OptiMem or not treated with trypsin.

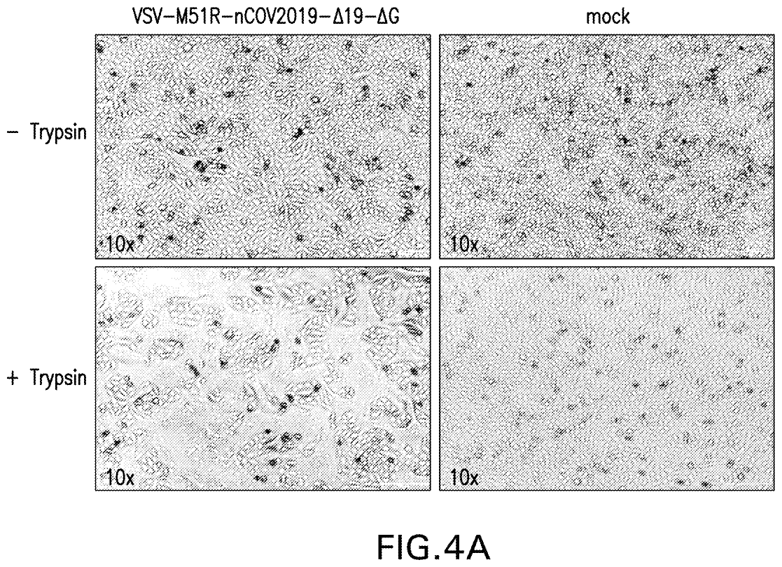

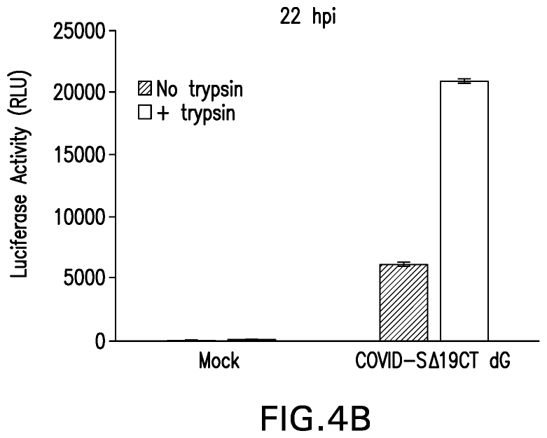

EnduRen luciferase substrate was added for luciferase signal detection. Fusion was assessed by measuring luciferase signal at 22 hours post infection. The data in FIGS. 4A-B indicate that variant 2 (VSV SARS-CoV-2 Δ19CT dG)-induced fusion can be detected using the Vero-DSP1-PuroNero-DSP2-Puro cells and that trypsin enhances cell fusion brought about by the variant 2 virus.

### Example 3: Safety and Immunogenicity of the Recombinant VSV Particles Expressing SARS-Cov-2 Spike (S) Glycoprotein in Cynomolgus Macaques

Recombinant VSV particles comprising SARS-CoV-2 dG (variant 1), SARS-CoV-2 Δ19CT dG (variant 2), SARS-CoV-2 VSV-G CT dG (variant 3), and/or SARS-CoV-2 dG generated with WT Kozak sequence (variant 4) are prepared as described above in Example 1 and used to determine the VSV particle's safety and immunogenicity in a cynomolgus macaque study using intramuscular (IM) and/or oral delivery. A saline control is used for comparison. Alternatively, the VSV particles are administered transnasally (IN) under anesthesia.

TABLE 5
Study Design, Dose, and Route Indicated (Intramuscular IM, Oral)

| GP | N | IMMUNIZATION | DOSE (TCID₅₀) | ROUTE |
|---|---|---|---|---|
| 1 | N = 2 | Saline | | Intramuscular |
| 2 | N = 2 | VSV Particles | 2e7 | Intramuscular |
| 3 | N = 2 | VSV Particles | 2e8 | Intramuscular |
| 4 | N = 2 | Saline | | Oral |
| 5 | N = 2 | VSV Particles | 2e7 | Oral |
| 6 | N = 2 | VSV Particles | 2e8 | Oral |

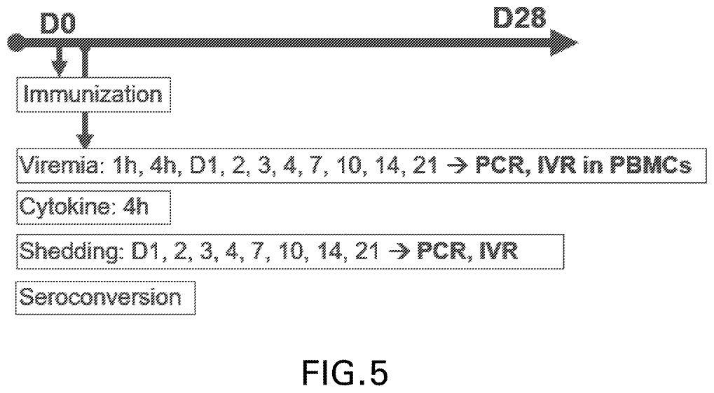

Physiological observations (e.g., viral viremia and shedding (e.g., blood/serum, nasal, oral, rectal, swabs), cytokine plasma levels, seroconversion, body weight, blood pressure, plasma oxygen levels, lung capacity, and body temperature), and visual observations (e.g., lesions, shivering, writhing, and piloerection) are carried out following the administration date. After day 28, the animals are euthanized for necropsy and histopathology of all tissues. FIG. 5 provides an example testing regimen. Seroconversion assays can include those listed in Table 6.

TABLE 6
Seroconversion Assays

| Sample | Seroconversion Assay |
|---|---|
| Serum | NtAb αVSV and αCoV2-S |
| | IgM and IgG ELISA against |
| | VSV-G and SARS-S |
| Nasal wash | NtAb Anti-SARS IgA ELISA |
| Tracheal/bronchial wash | NtAb Anti-SARS IgA ELISA |

Serological studies are also conducted (e.g., in an assay as depicted in Example 2), to demonstrate that the SARS-Cov-2 S glycoprotein expressing recombinant VSV particles are able to induce the formation of neutralizing antibodies against SARS-Cov-2.

The vaccine effect exhibited by the SARS-Cov-2 S glycoprotein expressing recombinant VSV particles demonstrate that the VSV constructs work as a vaccine against SARS-CoV-2, providing a protective effect against SARS-CoV-2.

### Example 4: Safety and Immunogenicity of the Recombinant VSV Particles Expressing SARS-Cov-2 Spike (S) Glycoprotein in Rhesus Macaques Followed by SARS-CoV2 Challenge

Recombinant VSV particles comprising SARS-CoV-2 dG (variant 1), SARS-CoV-2 Δ19CT dG (variant 2), SARS-CoV-2 VSV-G CT dG (variant 3), and/or SARS-CoV-2 dG generated with WT Kozak sequence (variant 4) are prepared as described above in Example 1 and used to determine the VSV particle's safety and immunogenicity in a rhesus macaque challenge study using intramuscular (IM) and/or oral delivery and a saline control for comparison. Alternatively, the VSV particles are administered intranasally (IN) under anesthesia. On day 28 following the administration of the VSV particles, the rhesus macaques are then challenged with SARS-CoV-2 intranasally (e.g., 10⁶ PFU).

TABLE 7
Study Design, Dose, and Route Indicated (Intramuscular IM, Oral)

| GP | N | IMMUNIZATION | DOSE (TCID₅₀) | ROUTE | CHALLENGE (D28) |
|---|---|---|---|---|---|
| 1 | N = 2 | Saline | | Intramuscular | SARS-CoV-2 1e6 PFU intranasal |
| 2 | N = 2 | VSV Particles | 2e7 | Intramuscular | SARS-CoV-2 1e6 PFU intranasal |
| 3 | N = 2 | VSV Particles | 2e8 | Intramuscular | SARS-CoV-2 1e6 PFU intranasal |
| 4 | N = 2 | Saline | | Oral | SARS-CoV-2 1e6 PFU intranasal |
| 5 | N = 2 | VSV Particles | 2e7 | Oral | SARS-CoV-2 1e6 PFU intranasal |
| 6 | N = 2 | VSV Particles | 2e8 | Oral | SARS-CoV-2 1e6 PFU intranasal |

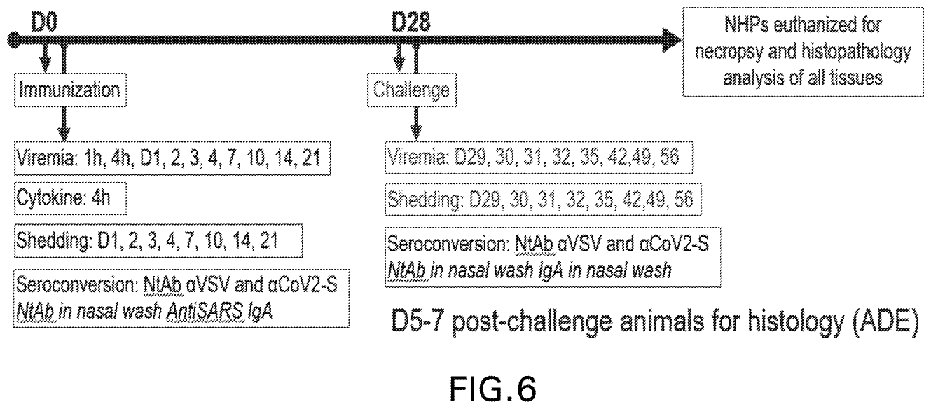

Physiological observations (e.g., viral viremia and shedding (e.g., blood/serum, nasal, oral, rectal, swabs), cytokine plasma levels, seroconversion, body weight, blood pressure, plasma oxygen levels, lung capacity, and body temperature), and visual observations (e.g., lesions, shivering, writhing, and piloerection) are carried out following the administration date. After 5 to 7 days post challenge, the animals are euthanized for necropsy and histopathology of all tissues. FIG. 6 provides an example testing regimen. Seroconversion assays include the same studies outlined in Example 3

Serological studies are also conducted (e.g., in an assay as depicted in Example 2), to demonstrate that the SARS-Cov-2 S glycoprotein expressing recombinant VSV particles are able to induce the formation of neutralizing antibodies against SARS-Cov-2.

The vaccine effect exhibited by the SARS-Cov-2 S glycoprotein expressing recombinant VSV particles demonstrate that the VSV constructs work as a vaccine against SARS-CoV-2, providing a protective effect against SARS-CoV-2.

### Example 5: Safety of the Recombinant VSV Particles Expressing SARS-Cov-2 Spike (S) Glycoprotein After Intrathalamic Injection in Rhesus Macaques

Recombinant VSV particles comprising SARS-CoV-2 dG (variant 1), SARS-CoV-2 Δ19CT dG (variant 2), SARS-CoV-2 VSV-G CT dG (variant 3), and/or SARS-CoV-2 dG generated with WT Kozak sequence (variant 4) are prepared as described above in Example 1 and used to determine the VSV particle's safety in a rhesus macaque study using intrathalamic (IT) delivery and a saline control for comparison.

TABLE 8
Study Design, Dose, and Route Indicated (Intrathalamic)

| GP | N | immunization | DOSE (TCID₅₀) | ROUTE |
|---|---|---|---|---|
| 1 | N = 2 | Saline | | Intrathalamic |
| 2 | N = 4 | VSV Particles | 2e7 | Intrathalamic |
| 3 | N = 4 | VSV Particles | 2e8 | Intrathalamic |

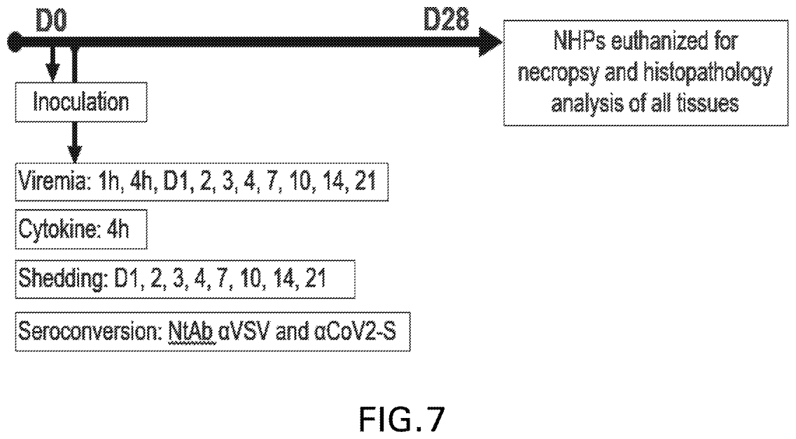

Physiological observations (e.g., viral viremia and shedding (e.g., blood/serum, nasal, oral, rectal, swabs), cytokine plasma levels, seroconversion, body weight, blood pressure, plasma oxygen levels, lung capacity, and body temperature), and visual observations (e.g., lesions, shivering, writhing, and piloerection) are carried out following the administration date. After day 28, the animals are euthanized for necropsy and histopathology of all tissues. FIG. 7 provides an example testing regimen. Seroconversion assays include the same studies outlined in Example 3.

Serological studies are also conducted (e.g., an assay as depicted in Example 2), to demonstrate that the SARS-Cov-2 S glycoprotein expressing recombinant VSV particles are able to induce the formation of neutralizing antibodies against SARS-Cov-2.

The vaccine effect exhibited by the SARS-Cov-2 S glycoprotein expressing recombinant VSV particles demonstrate that the VSV constructs work as a vaccine against SARS-CoV-2, providing a protective effect against SARS-CoV-2.

### Example 6: Safety, Transmissibility, and Immunogenicity of the Recombinant VSV Particles Expressing SARS-Cov-2 Spike (S) Glycoprotein in Pigs

Recombinant VSV particles comprising SARS-CoV-2 dG (variant 1), SARS-CoV-2 Δ19CT dG (variant 2), SARS-CoV-2 VSV-G CT dG (variant 3), and/or SARS-CoV-2 dG generated with WT Kozak sequence (variant 4) are prepared as described above in Example 1 and used to determine the VSV particle's safety, transmissibility, and immunogenicity in 4 week old Yorkshire cross pigs using intradermal snout scarification. The studies are conducted to assess (1) whether infection with the VSV particles results in clinical disease in pigs, (2) whether infection with the VSV particles results in virus shedding, or (3) whether the VSV particles are transmissible in natural host species.

TABLE 9
Study Design and Dose, and Route Indicated (Intradermal)

| GP | N | INOCULATION |
|---|---|---|
| 1 | N = 4 | 10⁶ TCID₅₀ VSV Particles |
| | | Intradermal inoculation on the snout |
| 2 | N = 4 | Non-inoculated contact pigs |

Transmissibility studies are also conducted wherein the inoculated group (GP 1) are housed with the non-inoculated group (GP 2). The non-inoculated group is tested to determine whether they developed viral shedding or neutralizing antibodies against SARS-Cov-2. An absence of seroconversion indicates absence of VSV transmission and vice versa.

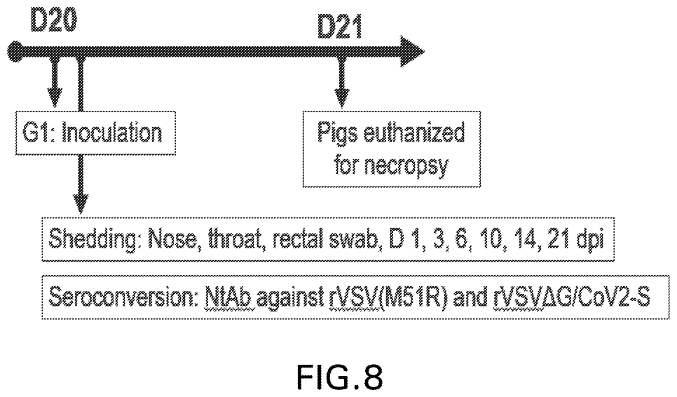

Physiological observations (e.g., viral viremia and shedding (e.g., blood/serum, nasal, oral, rectal, swabs), cytokine plasma levels, seroconversion, body weight, blood pressure, plasma oxygen levels, lung capacity, and body temperature), and visual observations (e.g., lesions, shivering, writhing, and piloerection) are carried out following the administration date. After day 21, the animals are euthanized for necropsy and histopathology of tissues. FIG. 8 provides an example testing regimen. Seroconversion assays include the same studies outlined in Example 3.

Serological studies are conducted (e.g., in an assay as depicted in Example 2), to demonstrate that the SARS-Cov-2 S glycoprotein expressing recombinant VSV particles are able to induce the formation of neutralizing antibodies against SARS-Cov-2.

The vaccine effect exhibited by the SARS-Cov-2 S glycoprotein expressing recombinant VSV particles demonstrate that the VSV constructs work as a vaccine against SARS-CoV-2, providing a protective effect against SARS-CoV-2.

### Example 7: Study of SARS-CoV-2 Vaccine in Humans

A phase I/II/III single-blinded, randomized, placebo controlled, multi-center study to determine efficacy, safety and immunogenicity of the recombinant VSV particles vaccine expressing SARS-Cov-2 S glycoprotein healthy adult volunteers aged 18-55 years is conducted. The vaccine is administered intramuscularly (IM) or subcutaneously (SC). Subjects are blinded and do not know if they have received the vaccine or the placebo.

Primary Outcome Measures:

The efficacy of the recombinant VSV particle vaccine against COVID-19 is assessed by, for example, determining the number of virologically confirmed (PCR positive) symptomatic cases (e.g., time frame: 6 months).

The safety of the recombinant VSV particle vaccine is assessed by, for example, determining the occurrence of serious adverse events (SAEs) (e.g., time frame: 6 months).

Cellular and humoral immunogenicity of the recombinant VSV particle vaccine is assessed via virus neutralizing antibody assays.

### Example 8: Safety and Immunogenicity of VSV-SARS2 Vaccine After Oral or Intramuscular Injection into Cynomolgus Macaques

VSV-SARS2 is a recombinant Indiana strain of Vesicular Stomatitis Virus whereby its G glycoprotein is replaced by the spike glycoprotein of SARS-CoV-2 with a deletion of 19 amino acids KFDEDDSEPVLKGVKLHYT (SEQ ID NO: 14) in the cytoplasmic tail. SARS-CoV-2 is the novel coronavirus that causes COVID-19. The goal of this study was to determine the safety and immunogenicity of two vaccine candidates, VSV-SARS2 and VSV-SARS2.G that is pseudotyped with the VSV.G glycoprotein (made in producer cells that express VSV.G glycoprotein) against SARS-CoV-2 virus. Furthermore, the relative safety and immunogenicity of VSV-SARS2.G after oral or intramuscular administration was also compared in this study. While intramuscular injection is a well-tested delivery route for vaccine delivery, the numbers of the SARS-CoV-2 receptor (ACE2) are limited on muscle cells. In contrast, abundant ACE receptors are found in the mucosal surfaces in the buccal cavity. Oral vaccination is more convenient and easy to administer to large populations, and does not require needles as required for intramuscular injection. Furthermore, oral immunization is more likely to induce mucosal IgA immunity, which can be important in protecting against SAR-CoV-2 infection (see e.g., Qiu et. al., “Mucosal Immunization of Cynomolgus Macaques with the VSVAG/ZEBOVGP Vaccine Stimulates Strong Ebola GP-Specific Immune Responses” PLoS One 2009; 4(5):e5547). Accordingly, the safety and immunogenicity of these two vaccine platforms were tested, and the routes of delivery (i.e., by direct oral administration (fluid form) and intramuscular) were compared.

### Study Design:

Six healthy cynomologus macaques were given the test articles as indicated in the table below. Test articles were given by intramuscular injection (1 ml) or given orally (5 ml or 12 ml) in sedated monkeys. Animals were monitored twice daily on Days 0-7 or as needed and then at least three times per week thereafter for clinical signs. Clinical specimens including complete blood counts, clinical chemistry, and body weights were recorded. Research correlatives included measurement of virus replication in the blood (viremia), virus shedding into mucosal surface or secretions, saliva, and importantly, the titers of anti-VSV or anti-SARS Cov2 antibodies by virus neutralization assay or by ELISA.

TABLE 10
Study Design Route Indicated (Intramuscular
IM, Oral), Test Article Given Day 0

| Group | 1 | 2 | 3 |
|---|---|---|---|
| NHP (n, Males) | 2 | 2 | 2 |
| Animal Study ID | CVAXE-1 | CVAXE-2 | CVAXE-3 |
| | CVAXE-4 | CVAXE-5 | CVAXE-6 |
| Route | Intramuscular | Oral | Oral |
| Test Article | VSV-SARS2.G | VSV-SARS2.G | VSV-SARS2 |
| Dose Given per | 1e8 TCID₅₀ | 1e8 TCID₅₀ | 1e7 TCID₅₀ |
| Animal | | | |

TABLE 11
Study Schedule for Interventions

| Study Day +/− | |
|---|---|
| 1-2 days | Event |
| Before Treatment | Obtained baseline hematological, chemistry |
| | parameters and baseline samples |
| | (blood, saliva, nasal, buccal, rectal swabs) |
| DO | Administered test articles |
| 1, 3, 7, 10, 14, | Blood: CBC, Chem, Viremia (Paxgene), Ab (serum) |
| 21, 28, 35 | and PBMC |
| | Saliva, buccal, nasal, rectal swab |
| | (virus shedding by PCR and infectious virus |
| | recovery, IVR) |
| 42 | Blood (Ab, PBMC for flow). Euthanize. Necropsy |
| | Splenocytes for ELISPOT T cell assays |

TABLE 12
Sample Collection at Scheduled Time Points

| | | | Antibodies | | |
|---|---|---|---|---|---|
| | | | (ELISA, and VNT) | | |
| | | Immune flow | IgA, IgM, IgG | Viral | Immune flow |
| | | and ELISPOT | against VSV | Shedding | and ELISPOT |
| Test | Viremia | assays | and SARS spike | (PCR, IVR) | assays |
| Harvest | Blood | Blood | Serum | Saliva | Splenocytes |
| Mode | | | | Buccal swabs | |
| | | | | Nasal swabs | |
| | | | | Rectal swabs | |
| | | | | Feces | |
| Blood | + | | | | |
| (Paxgene) | | | | | |
| Blood | | + | | | |
| (Heparin) | | (PBMC and plasma) | | | |
| Blood | | | + | | |
| (Clot tube) | | | | | |
| RNAlater | | | | + | |
| Frozen | | | | + | |
| Frozen | | | | | + |

### Assays:

Neutralizing Antibody Screen:
Sera were diluted to 1:50. In certain assays, sera were further serial diluted 2-fold to a maximum dilution of 1:6400.
Diluted samples were incubated with VSV-SARS-CoV-2-S-Δ19CT prior to infecting Vero cell monolayers. The Vero cell monolayer consisted of a mixture of two complimentary variants of a luciferase-based reporter system.

Viremia: VSV-N RNA in whole blood (10 draws)
Collected 1×2.5 ml blood in RNA Paxgene tubes.
Left on bench at room temp overnight before freezing in wire or plastic tube racks at <−65° C.

Peripheral blood mononuclear cells (PBMCs): Immune phenotyping (2 draws, Pre-tx, D42)
Collected 1×10 ml whole blood into heparin tubes Ficoll used to separate plasma (aliquot) and PBMCs (aliquot, freeze for immune flow)
Used for CD4/CD8 counts, and ELISPOT assays for VSV-N and SARS-spike T cells

Splenocytes: Immune phenotyping and ELISPOT assay (D42)
Harvested and isolated single cells
Ficoll used to separate cells if needed
Used for CD4/CD8 counts, and ELISPOT assays for VSV-N and SARS-spike T cells

Serum: VSV and SARS spike IgA, IgM, IgG subclass antibodies and virus neutralization test (Pre-tx, D4, 7, 11, 14, 21, 28, 35, 42, 9 draws)
Collected 10 ml blood into clot tubes
Obtained serum, aliquot into 500 μl per tube
Measured IgA, IgM, IgG subclasses and virus neutralization test

Serum: Multiplex cytokines (D1, 3)
Collected 5 ml blood into clot tubes (pre-treatment from above draw)
Obtained serum, aliquot into 500 μl per tube

Virus shedding: qRT-PCR (RNA protect) and infectious virus recovery (Frozen)
Saliva, nasal, buccal, rectal swabs and feces for qRT-PCR (where it was feasible)
Infectious virus recovery for VSV-N RNA or overlay on Vero cells (where it was feasible)
Limit of detection is 1:20.

Necropsy: RNA, Frozen, Formalin
Brain (frontal cortex, basal ganglia, thalamus, cerebellum, occipital cortex, olfactory bulb), spinal cord (cervical, thoracic, lumbar) CSF, oral mucosa, tongue, salivary glands, heart, lungs, spleen, liver, lymph nodes (axillary, inguinal, mesenteric, jejunal), gastrocnemius, skin/hair, sternum, diaphragm, pancreas, stomach, kidneys, adrenal glands, bone marrow, thymus, trachea, thyroid, parathyroid, esophagus, duodenum, jejunum, ileum, cecum, colon, rectum, bladder, reproductive organs (i.e., ovaries/uterus or testes), eyes, sciatic nerve, and nasal turbinates.

TABLE 13
Necropsy Sample Storage

| Test | RNA Later | Frozen | Formalin |
|---|---|---|---|
| Brain (*) | + | + | + |
| frontal cortex, | | | |
| occipital cortex, | | | |
| olfactory bulb | | | |
| Spinal cord (**) | + | + | + |
| CSF | − | + | − |
| Oral mucosal | + | + | + |
| Tongue | + | + | + |
| Salivary glands | + | + | + |
| Lungs | + | + | + |
| Liver | + | + | + |
| Spleen | + | + | + |
| Intestines | | | |
| Lymph nodes | + | + | + |
| Muscle | + | + | + |
| Other Tissues at | | | |
| Necropsy | | | |

### Results:

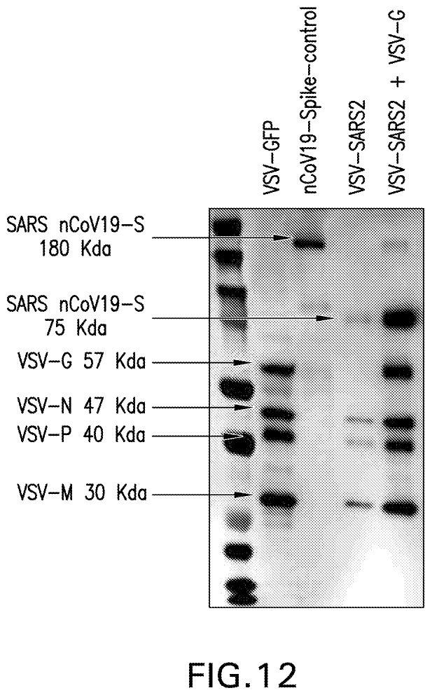

Western blot analysis demonstrated that both VSV-SARS2 and VSV-SARS2.G virions produced two bands corresponding to the full-length (180 kDa) and the proteolytically cleaved (75 kDa) glycoprotein (see FIG. 12). The Western blot analysis also showed the presence of VSV G, N, P, and M proteins in the parental VSV-GFP virions and VSV-SARS2.G, but only VSV N, P, and M proteins in the VSV-SARS2 virions.

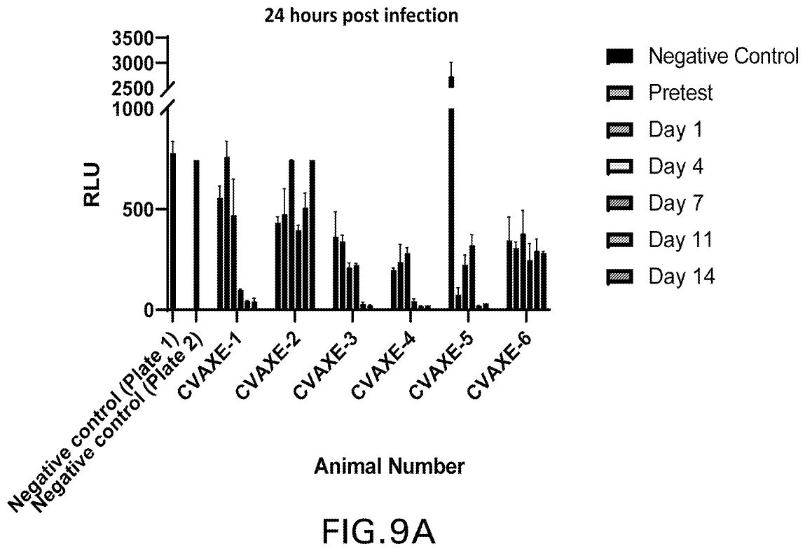

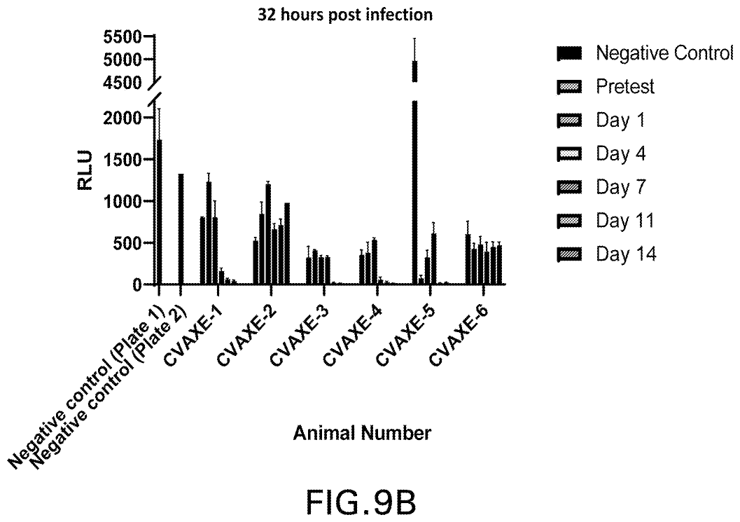

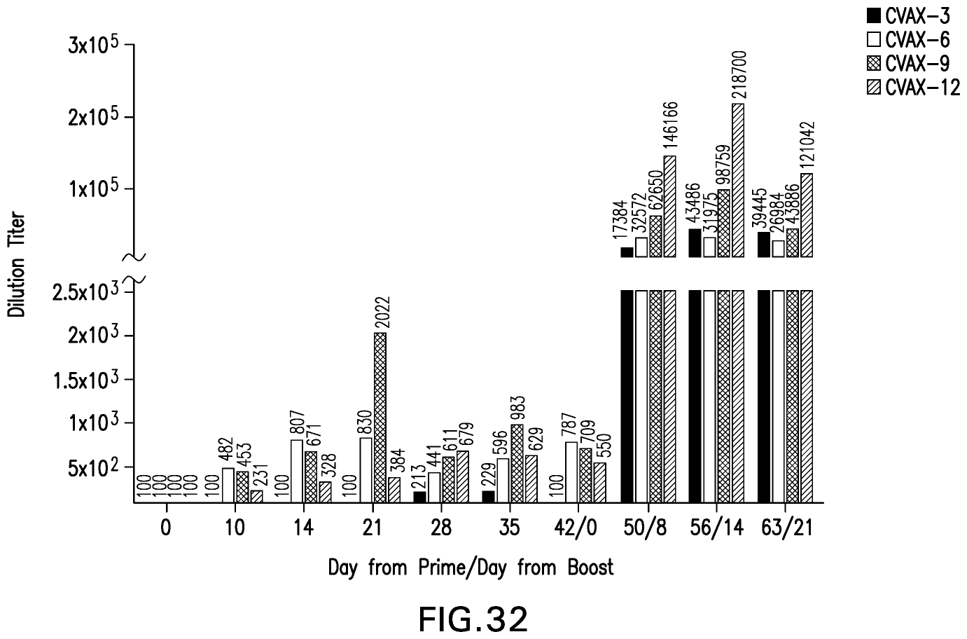

FIGS. 9A and 9B demonstrate a reduction of relative light units (RLU) starting at Day 7 (Animal CVAXE-1 and -4) and Day 11 (Animals CVAXE-3 and -5), which indicate the presence of neutralizing antibodies in the non-human primate (NHP) sera for 4 out of the 6 animals evaluated by Day 14. The NHP sera were diluted to the minimum recommended dilution established in the neutralizing antibody assay (1:50 for NHP serum matrix). Diluted samples were incubated with VSV-SARS-CoV-2-S-Δ19CT prior to infecting Vero cell monolayers. The Vero cell monolayer consisted of a mixture of two complimentary variants of a luciferase-based reporter system. Virus-induced cell fusion causes the production of a functional luciferase enzyme, and following incubation with substrate, chemiluminescent signal was read at both 24 hours post infection (hpi) (FIG. 9A) and 32 hpi (FIG. 9B).

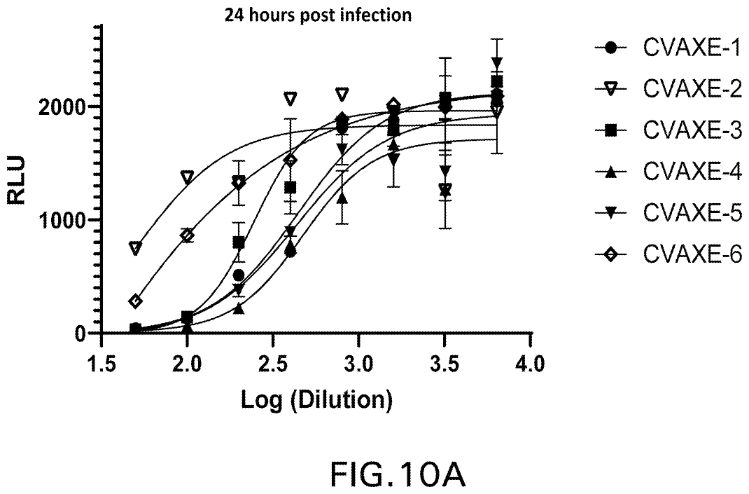

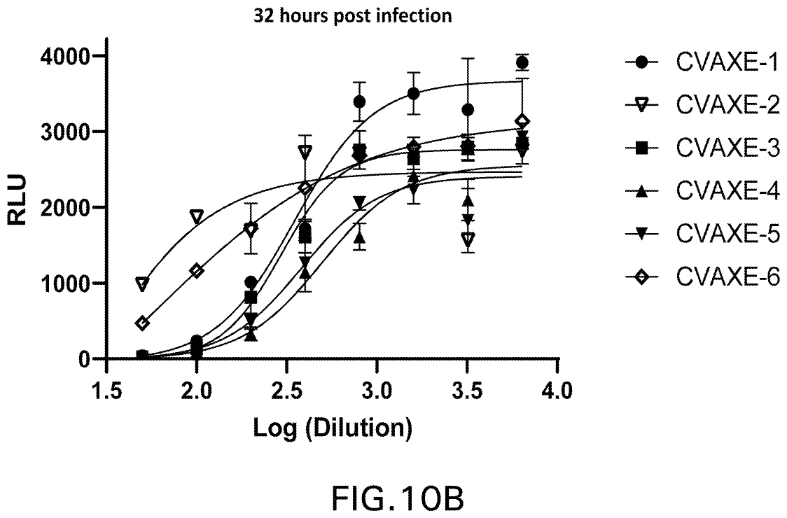

FIGS. 10A and 10B and Table 14 identify the EC₅₀ for each of the day 14 NHP serum samples, which serves to provide a measure of the level of neutralizing capacity for each of the serum samples by day 14. NHP sera were diluted starting at the minimum recommended dilution established in the neutralizing antibody assay (1:50 for NHP serum matrix) and further serial diluted 2-fold to a maximum dilution of 1:6400. Diluted samples were incubated with VSV-SARS-CoV-2-S-Δ19CT prior to infecting Vero cell monolayers. The Vero cell monolayer consisted of a mixture of two complimentary variants of a luciferase-based reporter system. Virus-induced cell fusion causes the production of a functional luciferase enzyme, and following incubation with substrate, chemiluminescent signal was read at both 24 hpi (FIG. 10A) and 32 hpi (FIG. 10B). Resulting relative light units (RLU) for each dilution were fitted to a 4-parameter logistic regression model, and the EC₅₀, meaning the dilution that resulted in the half maximal luciferase signal was determined.

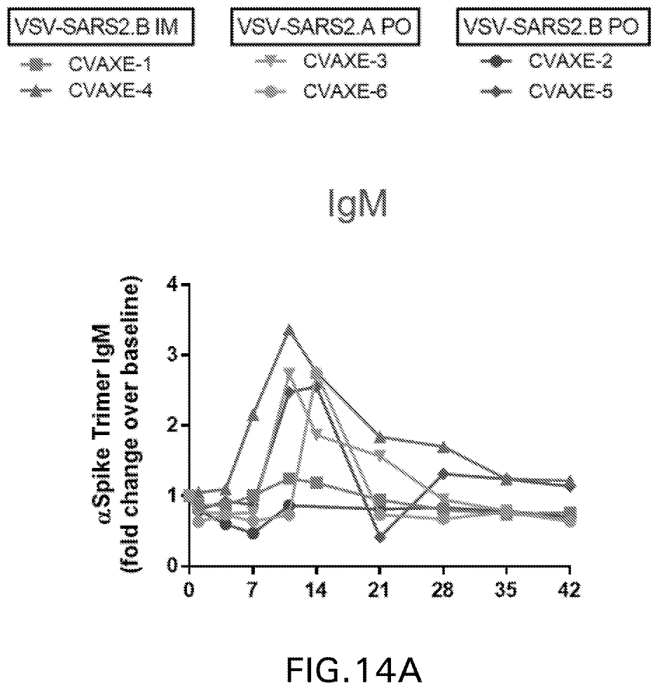

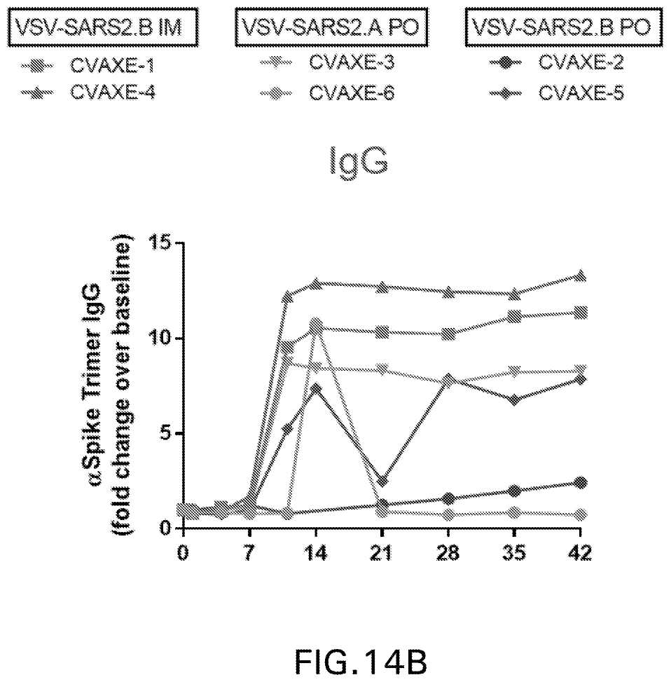

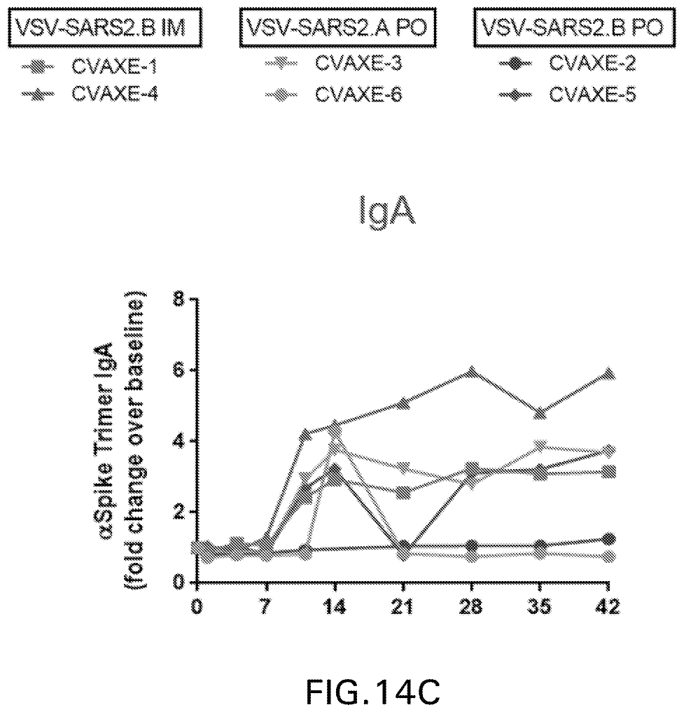

FIGS. 14A-14C provide anti-SARS-CoV-2 (Spike Trimer) antibody responses of IgM, IgG, and IgA from Day 0 to Day 42 for all animals. The data depicted in FIGS. 14A-14C were measured by ELISA; thus, these studies examined antibody binding and the time course of antibody response rather than neutralizing activity.

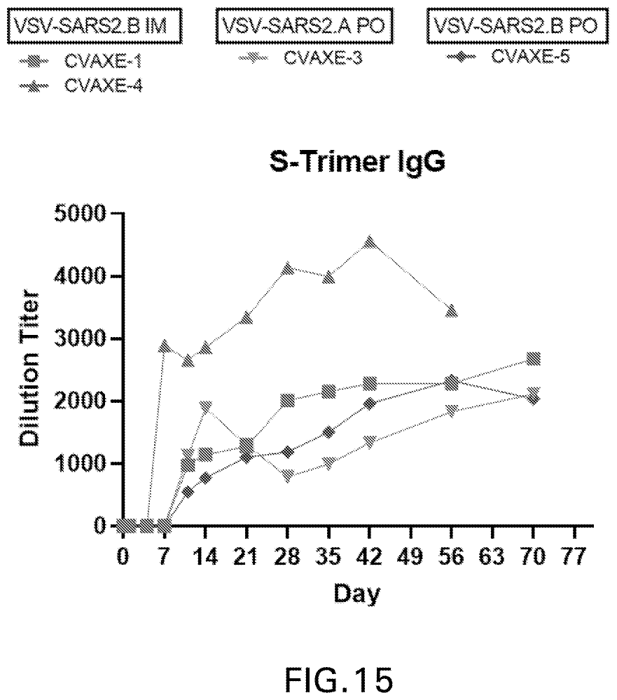

FIG. 15 provides the anti-SARS-CoV-2 spike trimer IgG dilution titer results for 4 animals up to Day 70, which exhibited seroconversion at Day 7 to Day 10. Data further demonstrated the magnitude of IgG response, and its long duration.

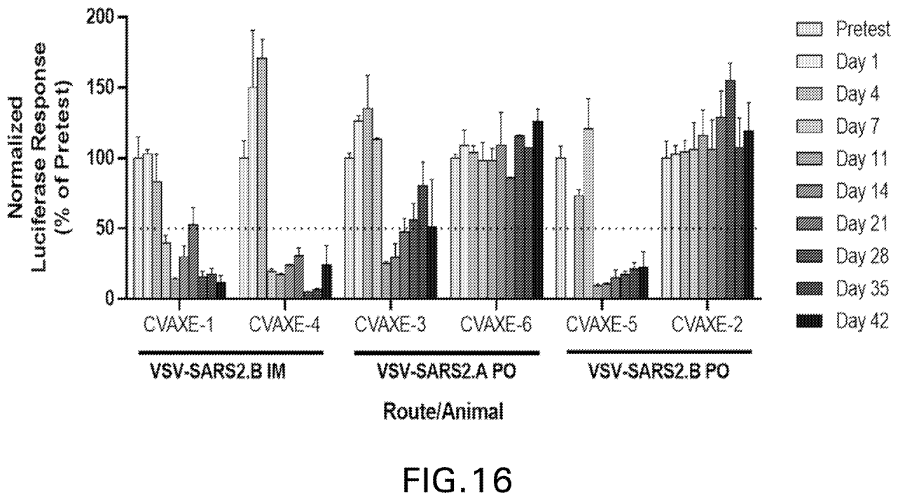

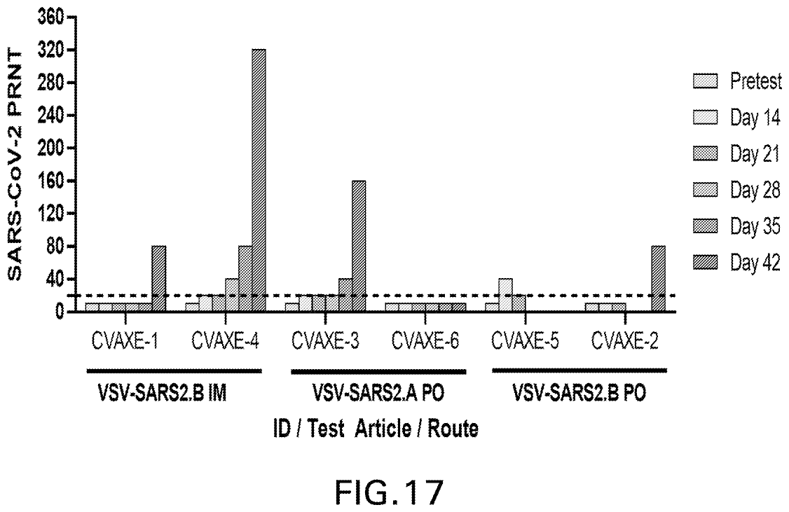

FIG. 16 examines the generation of neutralizing antibodies in vaccinated animals from Day 0 to Day 42, presented as normalized luciferase response as % of pretest levels. FIG. 17 examines neutralizing antibody activity as measured by a BSL3 clinical isolate of SARS-CoV-2, evaluated by PRNT assay. Data in FIG. 17 is supplementary to the data in FIG. 16, to further evaluate neutralizing antibody levels. CVAXE-4 (IM administration) and CVAXE-3 (Oral administration) both showed the highest levels of neutralizing activity, particularly at Day 35 and Day 42.

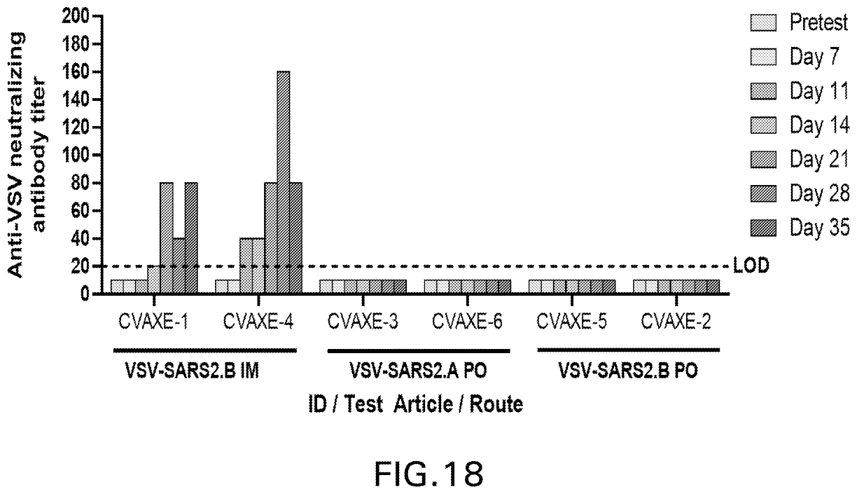

FIG. 18 examines anti-G mediated VSV neutralization. Data show the immunogenicity response against vaccine platform.

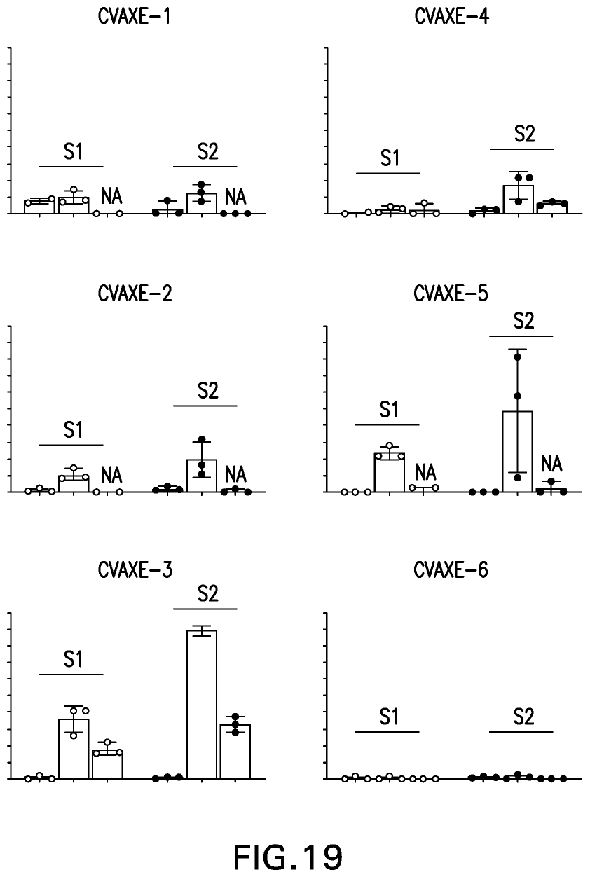

FIG. 19 examines T-cell mediated immune response by FluoroSpot assay. A peptide library of S1 domain and S2 domain peptides was used to evaluated IFN-gamma response, indicate of Th1 response, which peaked at Day 14 compared to Day 0 (Pre-immune) and Day 28 samples.

TABLE 14
Dilution titer determination at Day 14

| Animal Number | 24 hpi EC50 | 32 hpi EC50 |
|---|---|---|
| CVAXE-1 | 348.9 | 444.1 |
| CVAXE-2 | 23.85 | 52.13 |
| CVAXE-3 | 294.1 | 244.8 |
| CVAXE-4 | 498.5 | 473.4 |
| CVAXE-5 | 382.2 | 437.1 |
| CVAXE-6 | 59.82 | 17.87 |

As demonstrated in FIGS. 9A and 9B, four out of the six animals evaluated by day 14 developed neutralizing antibodies. Neutralizing antibodies were detected as early as 8 days after vaccination in the IM group and 11 days in the PO group and were still present at day 42 post-vaccination. All four animals with detectable neutralizing antibodies showed parallel increases in their IgG and IgM antibody titers against immobilized fragments of the SARS-CoV-2 spike glycoprotein (S1/S2, S1 subunit only, and RBD) and against the trimer form. Also, both of the IM vaccinated animals, but none of the orally vaccinated animals, developed anti-VSV G antibodies capable of neutralizing wild type VSV. On day 42 post vaccination, the two orally vaccinated animals that had failed to seroconvert were vaccinated by IM injection of 10⁷ or 10⁵ TCID₅₀ of the VSV-SARS2 virus. Both of these animals developed SARS-CoV-2 neutralizing antibodies within 14 days of the redosing. Data show a strong neutralizing antibody response for 4/6 animals by day 7 or 11, and strong neutralizing antibody presence maintained through day 42 for 3/6 animals. Anti-G mediated VSV neutralization appears to result from IM dose route, but it is unknown whether the oral dose route is capable of generating anti-G mediated VSV neutralization when G-pseudotyped virus is used. Thus, these results demonstrate that a single administration of the VSV-SARS2 vaccine, delivered either orally or intramuscularly, can result in the development of neutralizing antibodies. In fact, when delivered by intramuscular injection, neutralizing antibodies were present by day 7 (animals 1 and 4) and when delivered by the oral route, neutralizing antibodies were present by day 11 (animals 3 and 5).

Anti-SARS-CoV-2 (Spike Trimer) antibody response of IgM, IgG, and IgA from Day 0 to Day 42 for all animals (FIGS. 14A-14C) demonstrated that 4/6 animals showed seroconversion. As demonstrated in FIG. 15, the anti-SARS-CoV-2 spike antibody response (to S-trimer antigen) was sustained out to at least 70 days.

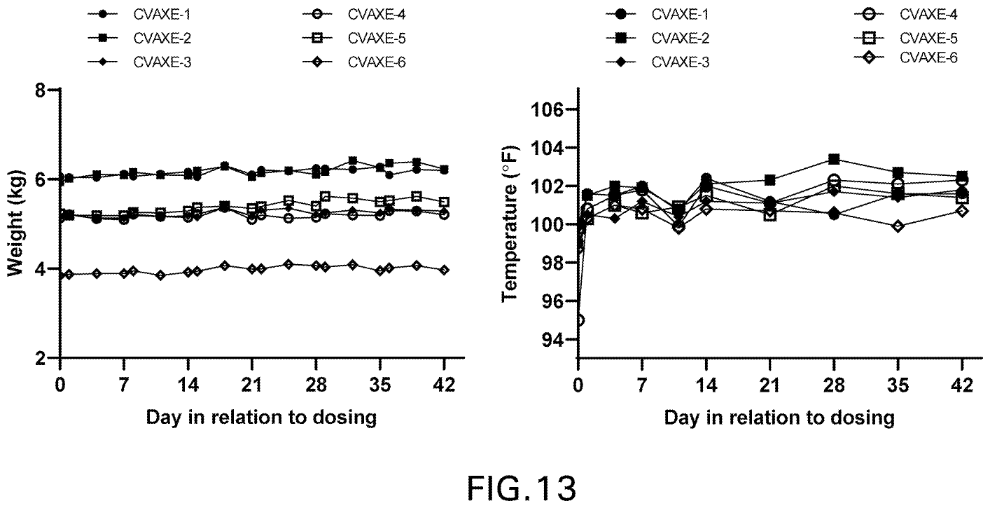

Additionally, as described above, animals were monitored closely for toxicity, viremia, virus shedding in urine and saliva, and for antibody response to the SARS-CoV-2 spike glycoprotein on days 1, 4, 8, 11, 14, 21, 28 and 42. Body temperature was mildly elevated during follow-up compared with baseline (98.6±1.8° F.) (see FIG. 13), and in 5 of the 6 animals, Grade 1 mucositis was observed but did not interfere with normal daily activities and was resolved without treatment. Episodic vomiting unrelated to the vaccine was observed, and was related to the sedation that was given to enable test article administration and sampling. Viremia was detected day 1 in both of the animals vaccinated by the IM route, but not at later time points and was never detected in orally vaccinated animals. Virus shedding in urine, saliva, feces, buccal, or nasal swabs was negative by PCR at all timepoints tested in all animals and no infectious virus was detected in any rectal, buccal or nasal swabs from any animal. Body weight was not affected at any of the timepoints (see FIG. 13). Thus, the VSV-SARS-CoV-2 viruses demonstrated a favorable safety profile.

### Example 9: Boosting with Oral Delivery of a Recombinant VSV Particle Vaccine Expressing SARS-Cov-2 S Glycoprotein

Recombinant VSV particles (e.g., variant 1, variant 2, variant 3, variant 4, and/or fragments or derivatives thereof (e.g., SEQ ID NO: 20 or SEQ ID NO: 22)) are prepared as described above in Example 1. The subject is administered a single intramuscular injection of the SARS-COV-2 vaccine mRNA-1273, BNT162a1, BNT162b1, BNT162b2, BNT162c2, or AZD1222 followed by intramuscular, oral, or mucosal (whether oral or intranasal) administration of a boosting dose of the recombinant VSV particle vaccine in the fluid form three months after administration of the intramuscular injection of the SARS-COV-2 vaccine. The recombinant VSV particle is administered intramuscularly, orally, or mucosally every three months following the initial boosting dose to prevent waning of immunity.

Primary Outcome Measures:

The efficacy of the boosting dose of the recombinant VSV particle vaccine against COVID-19 is assessed by, for example, determining the number of virologically confirmed (e.g., PCR positive) symptomatic cases (e.g., time frame: 6 months).

The safety of the boosting dose of the recombinant VSV particle vaccine is assessed by, for example, determining the occurrence of serious adverse events (SAEs) (e.g., time frame: 6 months).

Cellular and humoral immunogenicity of the boosting dose of the recombinant VSV particle vaccine is assessed via virus neutralizing antibody assays.

### Example 10: Functional Characterization of VSV-SARS-CoV2 Spike Virus

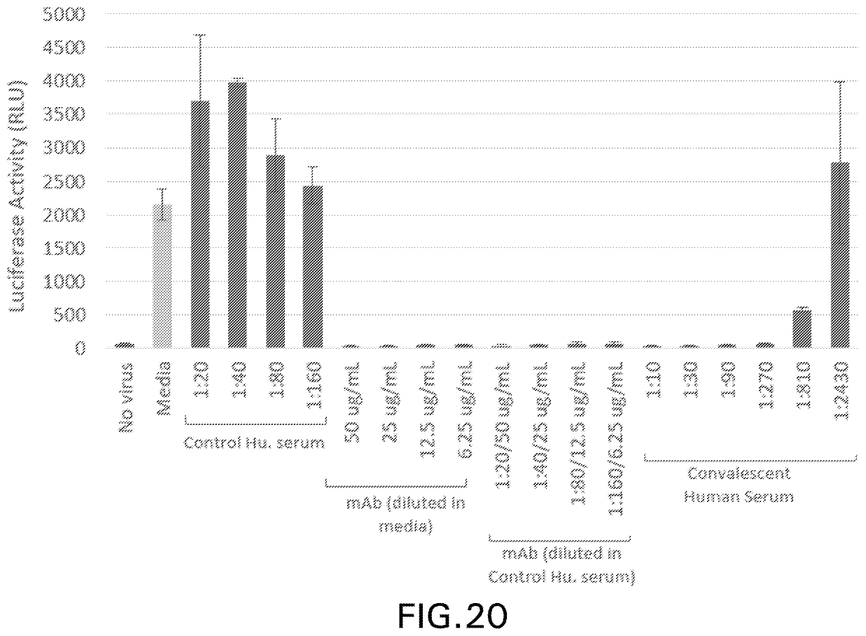

This example examines the neutralization of VSV-SARS2 (see Example 8) infectivity by anti-SARS-CoV-2 Spike monoclonal antibody and human convalescent serum. Media and dilutions of pre-immune serum had minimal impact on infectivity readout by fusion reporter cell lines (Luciferase from DSP-Veros) (see FIG. 20). A monoclonal antibody against SARS-CoV2 spike strongly inhibited infectivity of the virus, as did human convalescent serum sample.

### Example 11: Stability Studies of VSV-M_{WT}-SARS-CoV2-SΔ19 (VSV-SARS2), VSV-M_{WT}-SARS-CoV2-5Δ19+VSV-G (VSV-SARS2.G) and VSV-GFP

Samples all contain a base formulation of 50 mM Tris, 2 mM MgCl₂ at pH 7.4+/−the specified excipients (as indicated in the figures and drawings). 990 μl of base formulation+/−excipient was added to screw cap microtubes. 10 μl of VSV-SARS2 was added to the buffer and mixed by vortex. Samples were then placed in a box and either stored at 4° C. or frozen at −80° C. and thawed in RT water three times (i.e., three freeze/thaw cycles) as indicated below.

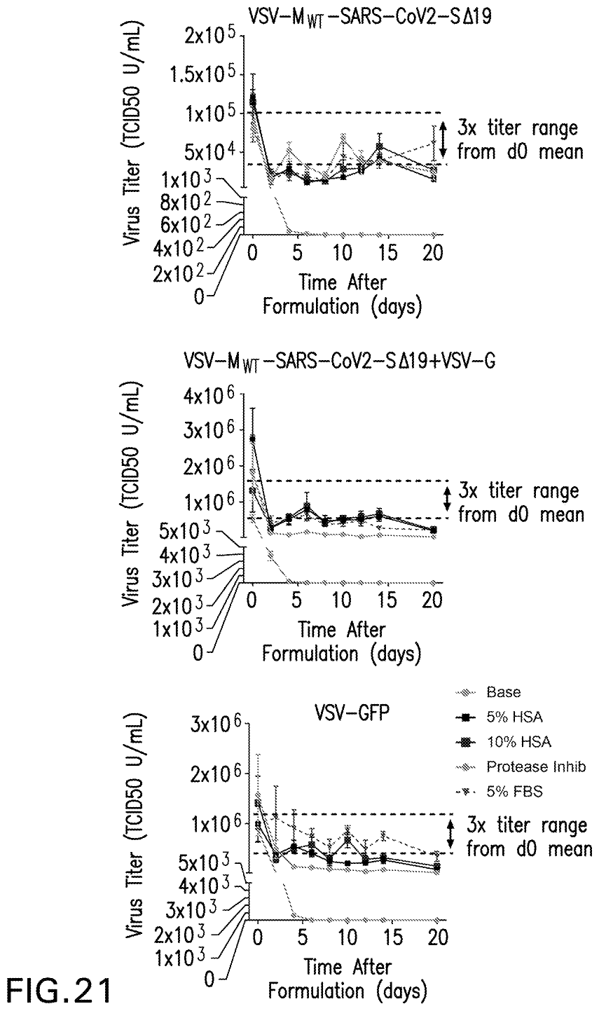

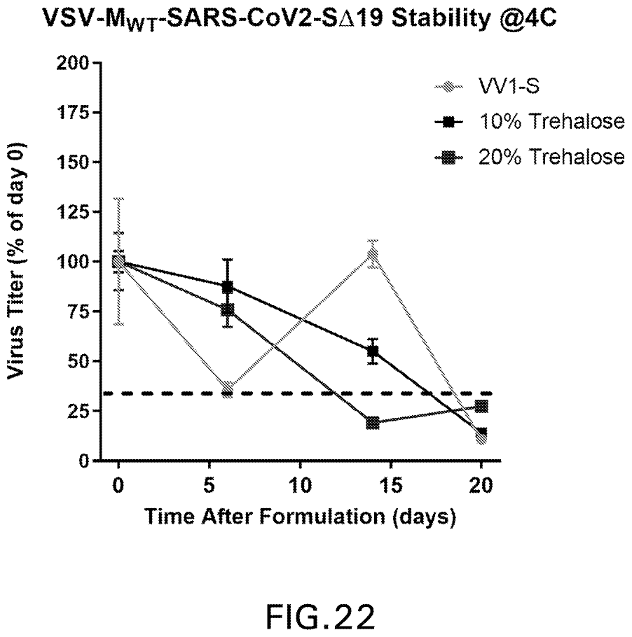

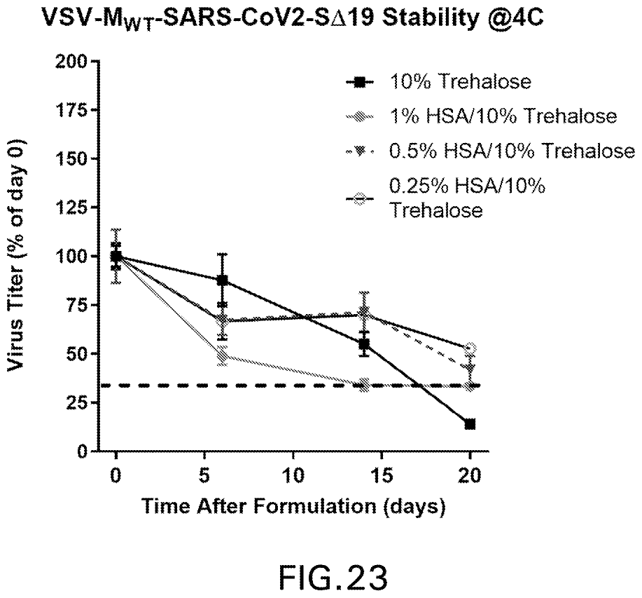

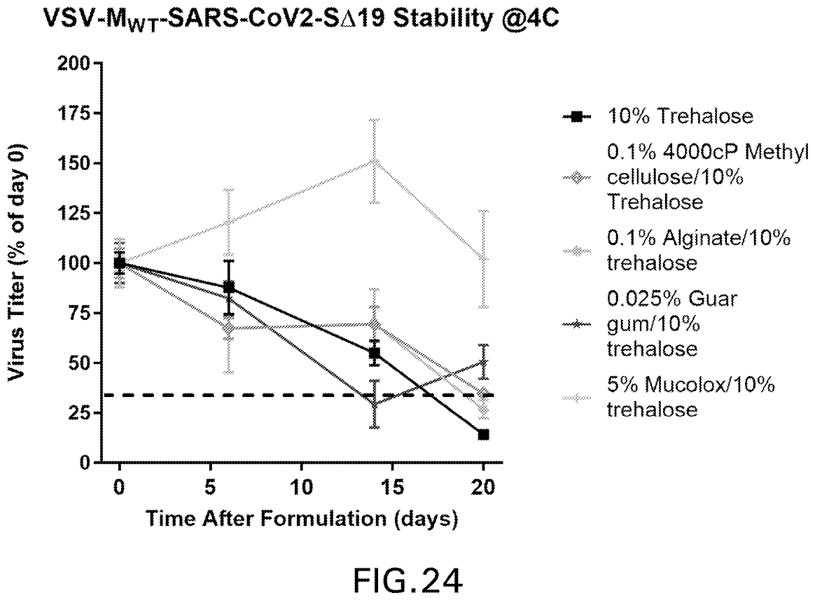

The first studies examine the stability of various vaccine formulations of VSV-SARS2 at 4° C. Samples stored at 4° C. were tested at day 0, 4, 6, 8, 10, 12, 14 and 20 (see FIG. 21) or at day 6, 14 and 20 (see FIGS. 22, 23 and 24). As is evident in FIG. 21, certain formulations remained within the 3×titer range from the day 0 mean after at least 14 days. Furthermore, FIGS. 22 and 23 show that certain formulations maintained an acceptable titer level (above the dotted line) up to at least day 14.

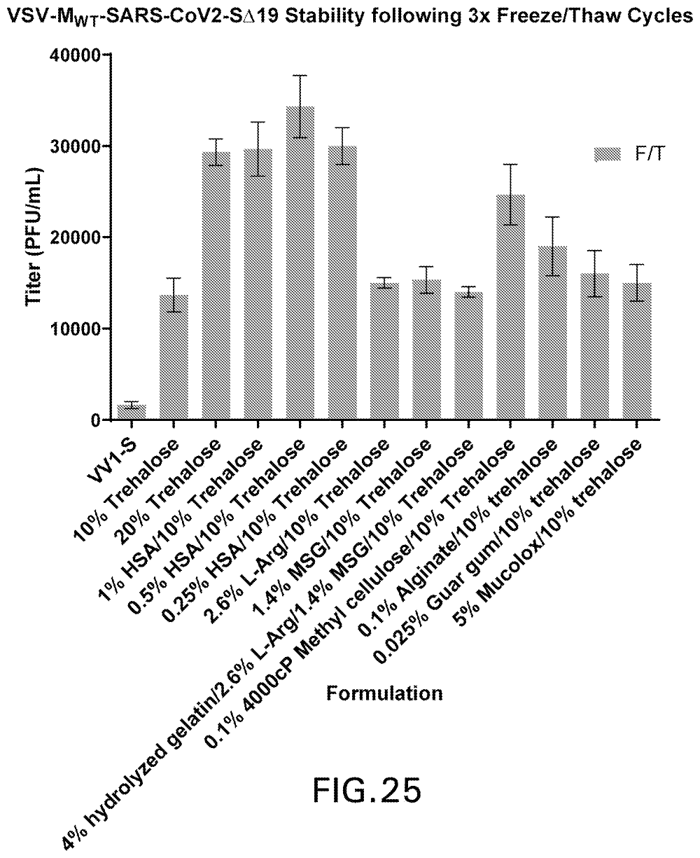

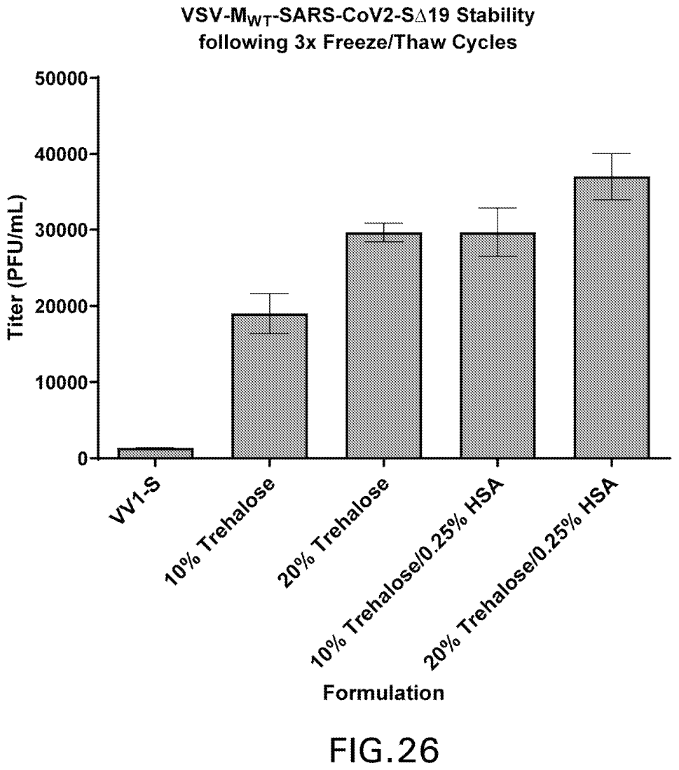

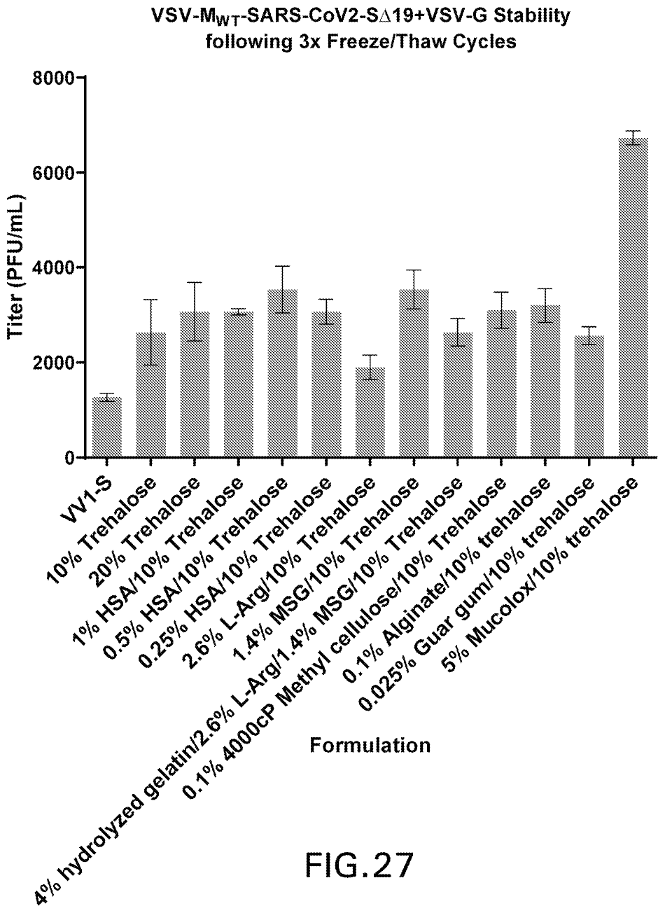

The second studies examine the stability of various vaccine formulations of VSV-SARS2 after multiple freeze/thaw cycles. Samples were tested after three freeze/thaw cycles (see FIGS. 25, 26 and 27). As shown, certain of the vaccine formulations maintained acceptable titer levels.

In a third set of studies, the stability of VSV-SARS2 was examined in mucoadhesive formulations. VSV-SARS2 was diluted to a target titer of about 200 PFU/ml in each formulation. OPTI-MEM™ was aspirated from the wells of a 24-well plate seeded the previous day with 2e5 Vero-His cells/well. 250 μl of the vaccine formulations were added to the wells and incubated at 37° C. for 5 minutes. The wells were washed twice with 400 μl OPTI-MEM and then 400 μl of OPTI-MEM was added to the wells. Each well was overlaid with OPTI-MEM/0.7% agarose with trypsin and incubated at 37° C. for 20-24 hours. The plates were fixed, stained and the plaques counted.

TABLE 15
Mucoadhesive Stability Studies

Viruses
VSV-M_{WT}-SARS-CoV2-SΔ19
VSV-M_{WT}-SARS-CoV2-SΔ19 + VSV-G

| | Concentration |
|---|---|
| Formulations | (0.25x, 0.05x, 0.01x) |
| 50 mM Tris-2 mM MgCl₂-Methyl cellulose | 0.5%, 0.1%, 0.02% |
| 25 cP | |
| 50 mM Tris-2 mM MgCl₂-Methyl cellulose | 0.5%, 0.1%, 0.02% |
| 4000 cP | |
| 50 mM Tris-2 mM MgCl₂-PCCA Mucolox | 25%, 5%, 1% |
| 50 mM Tris-2 mM MgCl₂-Sodium Alginate | 0.5%, 0.1%, 0.02% |
| (Sigma #W201502) | |
| 50 mM Tris-2 mM MgCl₂-Alginaic acid | 0.5%, 0.1%, 0.02% |
| sodium salt BioReagent (Siqma #71238) | |
| 50 mM Tris-2 mM MgCl₂ | N/A |

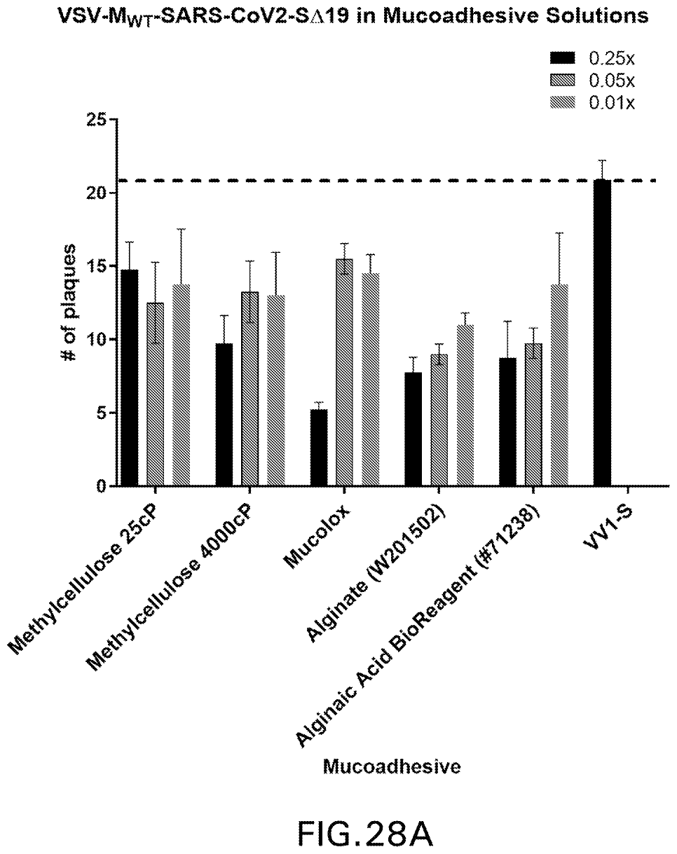

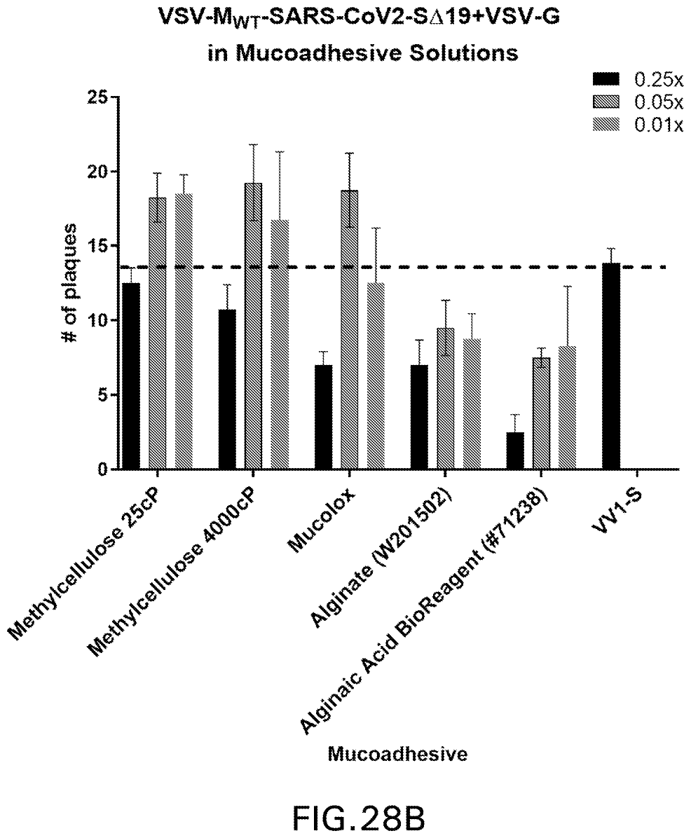

Samples were set up as shown in Table 15. The results are shown in FIG. 28A-B. The bar graph indicates the number of plaques counted compared to control (dotted line).

TABLE 16
Mucoadhesive Stability Studies

| | Concentration |
|---|---|
| Formulations | (0.25x, 0.05x, 0.01x) |
| 50 mM Tris-2 mM MgCl₂-20% Trehalose- | 0.5%, 0.1%, 0.02% |
| Methocel E50 | |
| 50 mM Tris-2 mM MgCl₂-20% Trehalose- | 0.5%, 0.1%, 0.02% |
| Methocel K4M | |
| 50 mM Tris-2 mM MgCl₂-20% Trehalose- | 25%, 5%, 1% |
| Methocel K15M | |
| 50 mM Tris-2 mM MgCl₂-20% Trehalose- | 0.5%, 0.1%, 0.02% |
| Methocel K100 | |
| 50 mM Tris-2 mM MgCl₂-20% Trehalose- | 0.5%, 0.1%, 0.02% |
| PCCA Mucolox | |
| 50 mM Tris-2 mM MgCl₂-20% Trehalose | N/A |

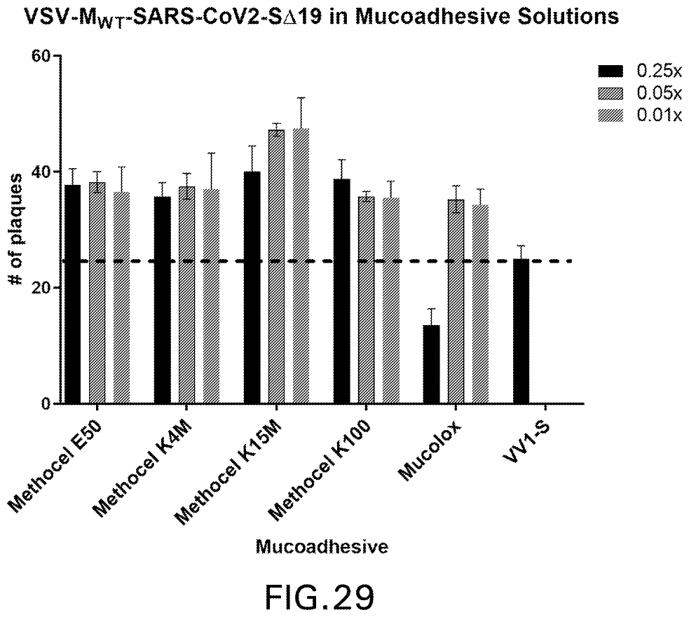

Samples were set up as shown in Table 16. The results are shown in FIG. 29. The bar graph indicates the number of plaques counted compared to control (dotted line).

### Example 12: Boosting with Oral Delivery of a Recombinant VSV Particle Vaccine Expressing SARS-CoV-2.G

VSV-SARS2.G vaccine incorporates both the SARS-CoV-2 spike glycoprotein and a plasmid-encoded VSV G protein into the viral envelopes. The recombinant VSV particles infect cells via the VSV G protein and SARS-CoV2 receptors, LDLR and ACE2, respectively. The viral progeny of infected cells lack the G protein and go on to infect cells exclusively via the ACE2 receptor.

A study was performed in cynomolgus macaques (NHPs) to test the efficacy of an orally administered boost using VSV-SARS.G vaccine. Twenty NHPs (CVAX-1 thru CVAX-20) received a primary vaccination with VSV-SARS2 (no G protein) according to Table 17.

TABLE 17
Study Design of Primary Vaccination

| Administration Route/Dose | Animal ID |
|---|---|
| IM/1e7 | 3, 6, 9, 12, 15, 18 |
| IM/1e5 | 19, 20 |
| Oral/1e7 | 1, 2, 4, 5, 7, 8, 10, 11, 13, 14, 16, 17 |

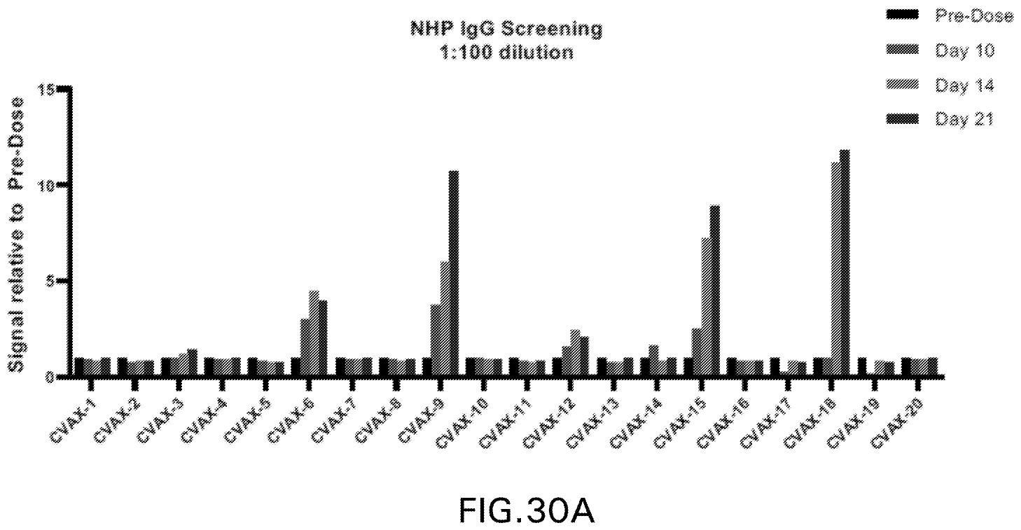

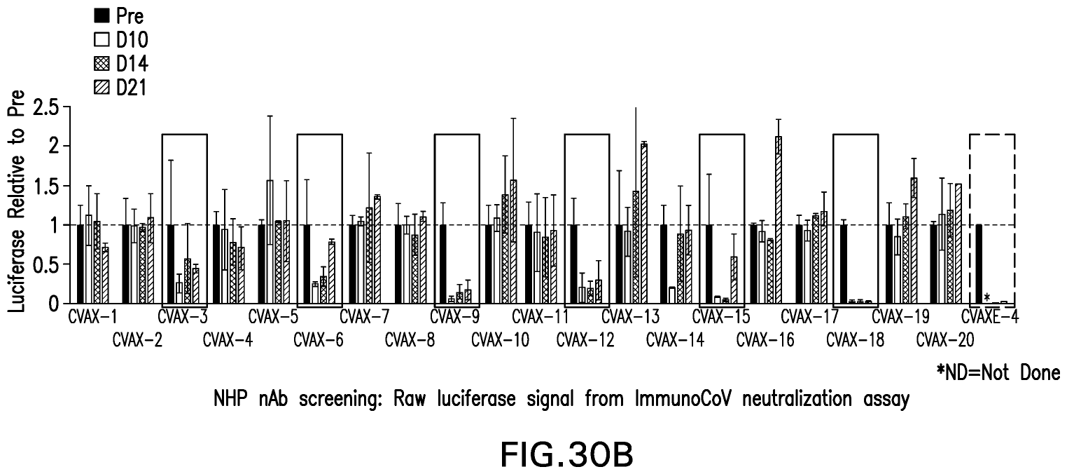

The NHPs were screened for COVID-19 neutralizing antibodies (nAb) pre-vaccination and days 10, 14 and 21 post vaccination. The results are shown in FIGS. 30A and 30B. The primary vaccination with VSV-SARS2 shows weak activity when administered IM and no activity when administered orally.

At day 42, an orally administered boost vaccination was delivered to CVAX-3, CVAX-6, CVAX-9 and CVAX-12 using a VSV-SARS2.G vaccine, specifically, MVB-14. CVAX-15 and CVAX-18 also received an orally administered boost vaccination with another VSV-SARS.G vaccine, CP-18. MVB-14 and CP-18 are both VSV-MWT-SARS-CoV2-SΔ19+VSV-G but were manufactured via slightly different processes. A comparison of MVB-14 and CP-18 is shown in Table 18.

TABLE 18
Comparison of MVB-14 and CP-18 Vaccines

| | MVB-14 | CP-18 |
|---|---|---|
| Cell line | Vero WHO | Vero WHO |
| Transfection Method | Electroporation | PEI (chemical) |
| DNA Plasmid Used* | pALD-VSV-G-K | pALD-VSV-G-K |
| Infection Virus** | 105.9.2.c.3.b PP4 | 20-VSVSARS-CP-16 |
| Titer | 2.58e8 | 7.10e6 |
| Formulation buffer/ | 50 mM Tris HCL | 50 mM Tris HCL |
| Excipient | (pH 7.4), 2 mM | (pH 7.4), 2 mM |
| | MgCl₂/10% | MgCl₂/10% |
| | Trehalose, 0.25% | Trehalose, 0.25% |
| | Human Serum | Human Serum |
| | Albumin | Albumin |

*the plasmid-encoded VSV G protein
**refers to the specific batch of the same virus utilized to manufacture the oral vaccine

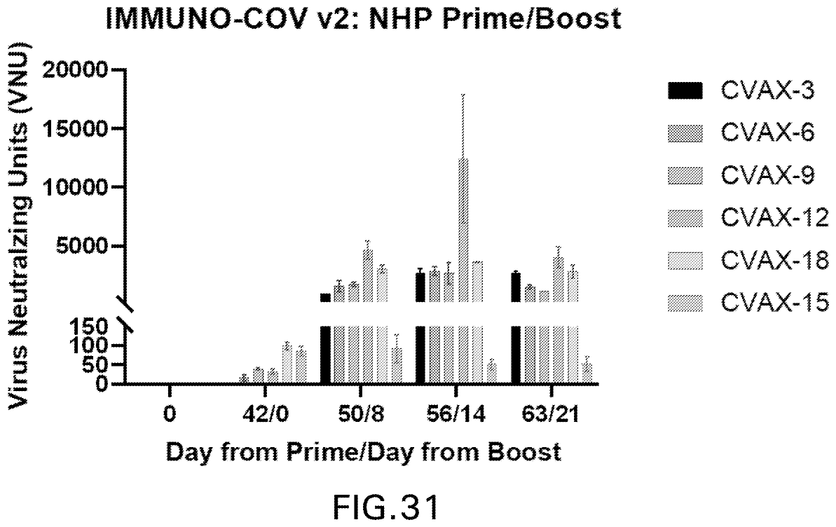

The MVB-14 boost vaccine was dosed at 1.25e9 and the CP-18 boost vaccine was dosed at 3.5e7. Responses were monitored by measuring virus neutralizing units (VNU) on days 50, 56, and 63. The results are shown in FIG. 31. The MVB-14 vaccine was highly successful at eliciting a boost response. The CP-18 vaccine elicited a response in only 1 of 2 animals. The most likely reason for the difference in effectiveness is due to the lower dose of the CP-18 vaccine administered versus the MVB-14 vaccine. Differences in preparation may also account for the difference in effectiveness such as, for example, transfection methods and/or the infection virus used. The actual VNUs are shown in Table 19.

TABLE 19
Tabular VNU Results

| | Days from Prime/Days from Boost | | | | |
|---|---|---|---|---|---|
| Animal | 0 | 42/0 | 50/8 | 56/14 | 63/21 |
| CVAX-3 | <Min | <Min | 946.21 | 2621.2 | 2696.7 |
| CVAX-6 | <Min | 16.042 | 1485.6 | 2801.3 | 1495 |
| CVAX-9 | <Min | 39.876 | 1680.8 | 2676.2 | 1441 |
| CVAX-12 | <Min | 33.161 | 4642.3 | 12401.43 | 3838.6 |
| CVAX-15 | <Min | 83.935 | 83.569 | 49.722 | 49.671 |
| CVAX-18 | <Min | 97.849 | 3010.3 | 3608.2 | 2752.7 |

Serum IgG binding to SARS-CoV-2 spike trimer was evaluated by ELISA. The results, shown in FIG. 32, show a major increase following the oral boost on day 42. It should be noted IgG binding to the VSV-G protein was not detected following orally administered vaccination (data not shown).

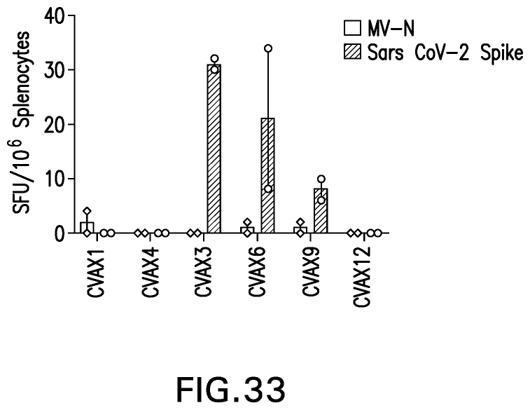

T cell recall responses for the SARS-CoV-2 Spike protein were also detected in three NHPs. INF-γ producing spots per million (SFU) splenocytes were determined by IFN-γ ELISPOT assay and the results are shown in FIG. 33.

### Example 13: Generation of Variants of SARS-CoV-2

The SARS-CoV-2 spike glycoprotein mutants were human codon optimized and synthesized with a deletion in the nucleotides encoding the C-terminal 19 amino acids (5-Δ19CT). The variants of SARS-CoV-2 were cloned into a plasmid encoding the VSV genome using the restriction sites MluI and NheI. The plasmid was sequence verified and used for infectious virus rescue on BHK-21 cells. VSV-G was co-transfected into the BHK-21 cells to facilitate rescue but was not present in subsequent passages of the virus.

### Example 14: Neutralization Escape for Developing SARS-CoV-2 Variant Vaccines

In light of the rapid spread of SARS-CoV-2 variants globally, there has been growing concern as to whether vaccines originally developed against the wild-type strain will be effective against these new variants. One approach to overcome the variant strains is by incorporating the mutations of the variants into the wild-type SARS-CoV-2 spike protein used to create the vaccine as exemplified in Example 13. However, subjects who have already been vaccinated with a wild-type SARS-CoV-2 spike protein vaccine will have developed neutralizing antibodies. Thus, if given a further variant vaccination or booster which is based on the wild-type SARS-CoV-2 spike protein, the neutralizing antibodies will neutralize the variant vaccine resulting in no immunity to the variants.

In order to prevent a variant vaccine from being neutralized by wild-type SARS-CoV-2 neutralizing antibodies, we are generating new recombinant VSV particles capable of escaping neutralization by those wild-type SARS-CoV-2 neutralizing antibodies. The spike protein mutations of the variants are then incorporated into neutralization-escape recombinant VSV particles resulting in recombinant VSV particle variants capable of mounting an immune response.

The neutralization-escape recombinant VSV particles are being generated by growing VSV-SARS2.G, as described herein, in the presence of neutralizing plasma from a subject that had been infected with wild-type COVID-19. Once neutralization-escape recombinant VSV particles are obtained, those particles will be used to generate variants as described in Example 13.

### Example 15: Non-VSV-G Pseudotyped VSV Particles

As described above, vaccination with VSV-SARS2.G may result in production of anti-VSV G antibodies capable of neutralizing wild-type VSV. The presence of these antibodies will likely affect the effectiveness of a boost. To overcome this potential problem, other non-VSV, rhabdovirus G proteins or fragments can be utilized for pseudotyping. Any functional rhabdovirus G protein or fragment that is not neutralized by anti-VSV G antibodies may be used.

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

### EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A recombinant virus particle, wherein the recombinant virus particle is a recombinant rhabdovirus particle comprising a rhabdovirus genome lacking a functional rhabdovirus glycoprotein G gene, and further wherein the recombinant virus particle comprises a polynucleotide sequence encoding at least one Severe Acute Respiratory Syndrome coronavirus 2 (SARS-CoV-2) spike (S) glycoprotein or fragment or derivative thereof.

2. The recombinant virus particle of claim 1, wherein the recombinant rhabdovirus particle is a recombinant vesiculovirus particle, wherein the recombinant vesiculovirus particle comprises a vesiculovirus genome lacking a functional vesiculovirus glycoprotein G gene.

3. The recombinant virus particle of claim 2, wherein the recombinant vesiculovirus particle is a recombinant vesicular stomatitis virus (VSV) particle, wherein the recombinant VSV particle comprises a VSV genome lacking a functional VSV glycoprotein G gene.

4. The recombinant virus particle of any one of claims 1-3, wherein the recombinant virus particle genome comprises the polynucleotide sequence encoding the at least one SARS-CoV-2 S glycoprotein or fragment or derivative thereof.

5. The recombinant virus particle of any one of claims 1-4, wherein the recombinant virus particle comprises the SARS-CoV-2 S glycoprotein or fragment or derivative thereof on the viral envelope.

6. The recombinant virus particle of any one of claims 1-5, wherein the recombinant virus particle is replication-competent.

7. The recombinant virus particle of any one of claims 1-6, wherein the SARS-CoV-2 S glycoprotein or fragment or derivative thereof is immunogenic and/or antigenic.

8. The recombinant virus particle of any one of claims 1-7, wherein the SARS-CoV-2 S glycoprotein or fragment or derivative thereof is capable of targeting a receptor on a host cell.

9. The recombinant virus particle of claim 8, wherein the targeting of the receptor results in the recombinant virus infecting the host cell.

10. The recombinant virus particle of claim 8 or claim 9, wherein the receptor is an angiotensin converting enzyme 2 (ACE2).

11. The recombinant virus particle of any one of claims 1-10, wherein the SARS-CoV-2 S glycoprotein comprises the amino acid sequence of SEQ ID NO: 1 or an amino acid sequence that has at least 80% sequence identity to SEQ ID NO: 1.

12. The recombinant virus particle of claim 11, wherein the polynucleotide sequence encoding the SARS-CoV-2 S glycoprotein comprises SEQ ID NO: 2 or a polynucleotide sequence that has at least 80% sequence identity to SEQ ID NO: 2.

13. The recombinant virus particle of any one of claims 1-10, wherein the recombinant virus particle comprises the fragment or derivative of the SARS-CoV-2 S glycoprotein, wherein the fragment or derivative of the SARS-CoV-2 S glycoprotein results in a more fusogenic recombinant virus particle as compared to a comparable recombinant virus particle comprising a full-length wild-type SARS-CoV-2 spike glycoprotein.

14. The recombinant virus particle of any one of claim 1-10 or 13, wherein the recombinant virus particle comprises the fragment or derivative of the SARS-CoV-2 S glycoprotein, wherein the fragment or derivative of the SARS-CoV-2 S glycoprotein results in a more lytic recombinant virus particle as compared to a comparable recombinant virus particle comprising a full-length wild-type SARS-CoV-2 spike glycoprotein.

15. The recombinant virus particle of claim 13 or 14, wherein the fragment or derivative of the SARS-CoV-2 S glycoprotein and the full-length wild-type SARS-CoV-2 spike glycoprotein are inserted into the same position in the virus genome of the respective virus particles.

16. The recombinant viruses particle of any one of claims 1-15, wherein the fragment or derivative of the SARS-CoV-2 S glycoprotein comprises a substitution of S247R, D614N, R685Q, or any combination thereof.

17. The recombinant viruses particle of any one of claims 1-16, wherein the fragment or derivative of the SARS-CoV-2 S glycoprotein comprises a substitution of N501Y, substitution of E484K, substitution of D614G, deletion of residues 69-70 or any combination thereof.

18. The recombinant viruses particle of any one of claims 1-17, wherein the fragment or derivative of the SARS-CoV-2 S glycoprotein comprises a mutation of Q⁶⁷⁷TNSPRRARSV⁶⁸⁷ to QTILRSV or QTNSPGSASSV.

19. The recombinant virus particle of any one of claim 1-10 or 13-18, wherein the virus genome encodes a fragment of the SARS-CoV-2 S glycoprotein.

20. The recombinant virus particle of claim 19, wherein the fragment comprises an S1 subunit, S2 subunit, and/or receptor-binding domain (RBD), or fragments or derivatives thereof, of the SARS-CoV-2 S glycoprotein.

21. The recombinant virus particle of claim 20, wherein the fragment comprises an RBD or an amino acid sequence that has at least 80% sequence identity to the RBD.

22. The recombinant virus particle of claim 20, wherein the fragment consists of the RBD.

23. The recombinant virus particle of claim 19, wherein the fragment is a C-terminally truncated SARS-CoV-2 S glycoprotein.

24. The recombinant virus particle of claim 23, wherein the C-terminally truncated SARS-CoV-2 S glycoprotein comprises a deletion of one to 30 amino acids from the C-terminus of the SARS-CoV-2 S glycoprotein.

25. The recombinant virus particle of claim 24, wherein the C-terminally truncated SARS-CoV-2 S glycoprotein comprises a 19 amino acid deletion from the C-terminus of the of SARS-CoV-2 S glycoprotein.

26. The recombinant virus particle of claim 25, wherein the C-terminally truncated SARS-CoV-2 S glycoprotein comprises or consists of the amino acid sequence of SEQ ID NO: 3 or an amino acid sequence that has at least 80% sequence identity to SEQ ID NO: 3.

27. The recombinant virus particle of claim 26, wherein the polynucleotide sequence encoding the C-terminally truncated SARS-CoV-2 S glycoprotein comprises or consists of SEQ ID NO: 4 or a polynucleotide sequence that has at least 80% sequence identity to SEQ ID NO: 4.

28. The recombinant virus particle of claim 25, wherein the C-terminally truncated SARS-CoV-2 S glycoprotein comprises or consists of the amino acid sequence of SEQ ID NO: 20 or an amino acid sequence that has at least 80% sequence identity to SEQ ID NO: 20.

29. The recombinant virus particle of claim 28, wherein the polynucleotide sequence encoding the C-terminally truncated SARS-CoV-2 S glycoprotein comprises or consists of SEQ ID NO: 21 or a polynucleotide sequence that has at least 80% sequence identity to SEQ ID NO: 21.

30. The recombinant virus particle of claim 25, wherein the C-terminally truncated SARS-CoV-2 S glycoprotein comprises or consists of the amino acid sequence of SEQ ID NO: 22 or an amino acid sequence that has at least 80% sequence identity to SEQ ID NO: 22.

31. The recombinant virus particle of any one of claim 1-10, or 13-18, wherein the derivative of the SARS-CoV-2 S glycoprotein is a SARS-CoV-2 S fusion protein.

32. The recombinant virus particle of claim 31, wherein the SARS-CoV-2 S fusion protein is a fusion between a SARS-CoV-2 S glycoprotein, or fragment or derivative thereof, and a protein the enables viral entry.

33. The recombinant virus particle of claim 32, wherein the protein that enables viral entry is a non-SARS-CoV-2 fusogen or fragment or derivative thereof.

34. The recombinant virus particle of claim 33, wherein the fusogen is a VSV glycoprotein (G) protein or fragment or derivative thereof.

35. The recombinant virus particle of claim 34, wherein the fragment of the VSV G protein is a VSV G protein cytoplasmic tail.

36. The recombinant virus particle of claim 35, wherein the VSV G protein cytoplasmic tail comprises SEQ ID NO: 15 or an amino acid sequence that has at least 80% sequence identity to SEQ ID NO: 15.

37. The recombinant virus particle of claim 35, wherein the SARS-CoV-2 S fusion protein comprises the amino acid sequence of SEQ ID NO: 5 or an amino acid sequence that has at least 80% sequence identity to SEQ ID NO: 5.

38. The recombinant virus particle of claim 37, wherein the polynucleotide sequence encoding the SARS-CoV-2 S fusion protein comprises SEQ ID NO: 6 or a polynucleotide sequence that has at least 80% sequence identity to SEQ ID NO: 6.

39. The recombinant virus particle of any one of claims 1-38, wherein the polynucleotide that encodes the at least one SARS-CoV-2 S protein or fragment or derivative thereof is inserted within the virus G gene.

40. The recombinant virus particle of any one of claims 1-38, wherein the virus G gene is replaced by a polynucleotide encoding the at least one SARS-CoV-2 S protein or fragment or derivative thereof.

41. The recombinant virus particle of any one of claims 1-38, wherein the polynucleotide that encodes the at least one SARS-CoV-2 S protein or fragment or derivative thereof is inserted within a non-essential portion of the recombinant virus genome.

42. The recombinant virus particle of any one of claims 3-41, wherein the genome of the recombinant VSV particle comprises genes encoding VSV nucleoprotein (N), VSV phosphoprotein (P), and VSV large protein (L) proteins, or functional fragments or derivatives thereof.

43. The recombinant virus particle of any one of claims 3-42, wherein the genome of the recombinant VSV particle encodes a wild-type VSV matrix (M) protein.

44. The recombinant virus particle of claim 43, wherein the VSV M protein comprises the amino acid sequence of SEQ ID NO: 9 or an amino acid sequence that has at least 80% sequence identity to SEQ ID NO: 9.

45. The recombinant virus particle of claim 44, wherein the polynucleotide sequence encoding the VSV M protein comprises SEQ ID NO: 10 or a polynucleotide sequence that has at least 80% sequence identity to SEQ ID NO: 10.

46. The recombinant virus particle of any one of claims 3-42, wherein the genome of the recombinant VSV particle encodes a mutant VSV M protein.

47. The recombinant virus particle of claim 46, wherein the mutant VSV M protein comprises a mutation at methionine (M) 51.

48. The recombinant virus particle of claim 47, wherein the mutation is from methionine (M) to arginine (R).

49. The recombinant virus particle of claim 48, wherein the mutant VSV M protein comprises the amino acid sequence of SEQ ID NO: 7 or an amino acid sequence that has at least 80% sequence identity to SEQ ID NO: 7.

50. The recombinant virus particle of claim 49, wherein the polynucleotide sequence encoding the mutant VSV M protein comprises SEQ ID NO: 8 or a polynucleotide sequence that has at least 80% sequence identity to SEQ ID NO: 8.

51. The recombinant virus of claim 46, wherein the mutant VSV M protein comprises a deletion at methionine (M) 51.

52. A polynucleic acid comprising a polynucleotide sequence encoding a rhabdovirus nucleoprotein (N), a rhabdovirus phosphoprotein (P), and a rhabdovirus large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant rhabdovirus particle.

53. A polynucleic acid comprising a polynucleotide sequence encoding a vesiculovirus nucleoprotein (N), a vesiculovirus phosphoprotein (P), and a vesiculovirus large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant vesiculovirus particle.

54. A polynucleic acid comprising a polynucleotide sequence encoding vesicular stomatitis virus (VSV) nucleoprotein (N), a VSV phosphoprotein (P), and a VSV large protein (L), or functional fragments or derivatives thereof, and encoding a Severe Acute Respiratory Syndrome coronavirus 2 (SARS-CoV-2) spike (S) glycoprotein or fragment or derivative thereof, for expression on the viral envelope of a recombinant VSV particle.

55. The polynucleic acid of any one of claims 52-54 wherein the SARS-CoV-2 S glycoprotein or fragment or derivative thereof is immunogenic.

56. The polynucleic acid of any one of claims 52-55, wherein the SARS-CoV-2 S glycoprotein or fragment or derivative thereof is capable of targeting a SARS-CoV-2 spike protein receptor on a host cell comprising.

57. The polynucleic acid of claim 56, wherein the targeting of the receptor results in the recombinant virus particle infecting the host cell.

58. The polynucleotide of claim 56 or claim 57, wherein the receptor is an angiotensin converting enzyme 2 (ACE2).

59. The polynucleotide of any one of claims 52-58, wherein the SARS-CoV-2 S glycoprotein comprises the amino acid sequence of SEQ ID NO: 1 or an amino acid sequence that has at least 80% sequence identity to SEQ ID NO: 1.

60. The polynucleotide of claim 59, wherein the polynucleotide sequence encoding the SARS-CoV-2 S glycoprotein comprises SEQ ID NO: 2 or a polynucleotide sequence that has at least 80% sequence identity to SEQ ID NO: 2.

61. The polynucleotide of any one of claims 52-58, wherein the polynucleotide sequence encodes a fragment of the SARS-CoV-2 S glycoprotein.

62. The polynucleotide of claim 61, wherein the fragment comprises an 51 subunit, S2 subunit, and/or receptor-binding domain (RBD), or fragments or derivatives thereof, of the SARS-CoV-2 S glycoprotein.

63. The polynucleotide of claim 62, wherein the fragment comprises an RBD or an amino acid sequence that has at least 80% sequence identity to the RBD derivatives thereof.

64. The polynucleotide of claim 62, wherein the fragment consists of the RBD.

65. The polynucleotide of claim 61, wherein the fragment is a C-terminally truncated SARS-CoV-2 S glycoprotein.

66. The polynucleotide of claim 65, wherein the C-terminally truncated SARS-CoV-2 S glycoprotein comprises a deletion of one to 30 amino acids from the C-terminus of the SARS-CoV-2 S glycoprotein.

67. The polynucleotide of claim 66, wherein the C-terminally truncated SARS-CoV-2 S glycoprotein comprises a 19 amino acid deletion from the C-terminus of the of SARS-CoV-2 S glycoprotein.

68. The polynucleotide of claim 67, wherein the C-terminally truncated SARS-CoV-2 S glycoprotein comprises the amino acid sequence of SEQ ID NO: 3 or an amino acid sequence that has at least 80% sequence identity to SEQ ID NO: 3.

69. The polynucleotide of claim 68, wherein the polynucleotide sequence encoding the C-terminally truncated SARS-CoV-2 S glycoprotein comprises SEQ ID NO: 4 or a polynucleotide sequence that has at least 80% sequence identity to SEQ ID NO: 4.

70. The polynucleotide of claim 67, wherein the C-terminally truncated SARS-CoV-2 S glycoprotein comprises the amino acid sequence of SEQ ID NO: 20 or an amino acid sequence that has at least 80% sequence identity to SEQ ID NO: 20.

71. The polynucleotide of claim 70, wherein the polynucleotide sequence encoding the C-terminally truncated SARS-CoV-2 S glycoprotein comprises SEQ ID NO: 21 or a polynucleotide sequence that has at least 80% sequence identity to SEQ ID NO: 21.

72. The polynucleotide of claim 67, wherein the C-terminally truncated SARS-CoV-2 S glycoprotein comprises the amino acid sequence of SEQ ID NO: 22 or an amino acid sequence that has at least 80% sequence identity to SEQ ID NO: 22.

73. The polynucleotide of any one of claims 52-58, wherein the polynucleotide sequence encodes a derivative of the SARS-CoV-2 S glycoprotein, wherein the derivative is a SARS-CoV-2 S fusion protein.

74. The polynucleotide of claim 73, wherein the SARS-CoV-2 S fusion protein is a fusion between a SARS-CoV-2 S glycoprotein, or fragment or derivative thereof, and a non-SARS-CoV-2 fusogen or fragment or derivative thereof.

75. The polynucleotide of claim 74, wherein the fusogen is a VSV glycoprotein (G) protein or fragment or derivative thereof.

76. The polynucleotide of claim 75, wherein the VSV G protein fragment is a VSV G protein cytoplasmic tail.

77. The polynucleotide of claim 76, wherein the SARS-CoV-2 S fusion protein comprises the amino acid sequence of SEQ ID NO: 5 or an amino acid sequence that has at least 80% sequence identity to SEQ ID NO: 5.3′ to the SARS-CoV-2 S glycoprotein or fragment or derivative thereof

78. The polynucleotide of claim 77, wherein the polynucleotide sequence encoding the SARS-CoV-2 S fusion protein comprises SEQ ID NO: 6 or a polynucleotide sequence that has at least 80% sequence identity to SEQ ID NO: 6.

79. The polynucleotide of any one of claims 52-78, wherein the polynucleotide sequence further comprises a Kozak sequence polynucleotide.

80. The polynucleotide of claim 79, wherein the Kozak sequence is a wild-type Kozak sequence.

81. The polynucleotide of claim 80, wherein the wild-type Kozak sequence comprises SEQ ID NO: 11 or a derivative thereof.

82. The polynucleotide of claim 79, wherein the Kozak sequence is an optimized Kozak sequence.

83. The polynucleotide of claim 82, wherein the optimized Kozak sequence comprises SEQ ID NO: 12 or a derivative thereof.

84. The polynucleotide of any one of claims 54-83, wherein polynucleotide sequence further encodes a wild-type VSV matrix (M) protein.

85. The polynucleotide of claim 84, wherein the VSV M protein comprises the amino acid sequence of SEQ ID NO: 9 or an amino acid sequence that has at least 80% sequence identity to SEQ ID NO: 9.

86. The polynucleotide of claim 85, wherein the polynucleotide sequence encoding the VSV M protein comprises SEQ ID NO: 10 or a polynucleotide sequence that has at least 80% sequence identity to SEQ ID NO: 10.

87. The polynucleotide of any one of claims 54-83, wherein the polynucleotide sequence further encodes a mutant VSV M protein.

88. The polynucleotide of claim 87, wherein the mutant VSV M protein comprises a mutation at methionine (M) 51.

89. The polynucleotide of claim 88, wherein the mutation is from methionine (M) to arginine (R).

90. The polynucleotide of claim 89, wherein the mutant VSV M protein comprises the amino acid sequence of SEQ ID NO: 7 or an amino acid sequence that has at least 80% sequence identity to SEQ ID NO: 7.

91. The polynucleotide of claim 90, wherein the polynucleotide sequence encoding the mutant VSV M protein comprises SEQ ID NO: 8 or a polynucleotide sequence that has at least 80% sequence identity to SEQ ID NO: 8.

92. The polynucleotide of claim 87, wherein the mutant VSV M protein comprises at a deletion at methionine (M) 51.

93. The polynucleotide of any one of claims 52-92, wherein the polynucleotide sequence lacks a functional G protein gene.

94. The polynucleotide of claim 93, wherein the polynucleotide sequence encoding the SARS-CoV-2 S glycoprotein or fragment or derivative thereof is inserted within the virus G protein gene.

95. The polynucleotide of claim 93, wherein the virus G protein gene is replaced by the polynucleotide sequence encoding the SARS-CoV-2 S glycoprotein or fragment or derivative thereof.

96. The polynucleotide of any one of claims 52-92, wherein the polynucleotide sequence encoding the SARS-CoV-2 S glycoprotein or fragment or derivative thereof is inserted within a non-essential portion of the recombinant virus genome.

97. A composition comprising the polynucleotide of any one of claims 52-96 and a carrier and/or excipient.

98. A replication-competent recombinant vesicular stomatitis virus (VSV) particle comprising the polynucleotide of any one of claims 54-96.

99. A host cell comprising the recombinant virus particle of any one of claim 1-51 or 98.

100. A composition comprising the recombinant virus particle of any one of claim 1-51 or 98 and a carrier and/or excipient.

101. A pharmaceutical composition comprising the recombinant virus particle of any one of claim 1-51 or 98 and a pharmaceutically acceptable carrier and/or excipient.

102. A pharmaceutical composition comprising an inactivated recombinant virus particle of any one of claim 1, 2, 7-51, or 98 and a pharmaceutically acceptable carrier and/or excipient.

103. A immunogenic composition comprising an amount of the recombinant virus particle of any one of claim 1-51 or 98 effective to induce an immune response against a SARS-CoV-2 and a pharmaceutically acceptable carrier and/or excipient.

104. A immunogenic composition comprising an amount of the recombinant virus particle of any one of claim 1-51 or 98 effective to induce the formation of neutralizing antibodies against a SARS-CoV-2 and a pharmaceutically acceptable carrier and/or excipient.

105. A vaccine formulation comprising an amount of the recombinant virus particle of any one of claim 1-51 or 98 effective to induce an immune response against a SARS-CoV-2 and a pharmaceutically acceptable carrier and/or excipient.

106. A vaccine formulation comprising an amount of the recombinant virus particle of any one of claim 1-51 or 98 effective to induce the formation of neutralizing antibodies against a SARS-CoV-2 and a pharmaceutically acceptable carrier and/or excipient.

107. The vaccine formulation of claim 105 or claim 106, wherein the formulation is stable at 4° C.

108. The vaccine formulation of any one of claims 105-107, wherein the formulation increases the amount of time the recombinant virus particles of any one of claim 1-51 or 98 remain viable at 4° C.

109. The vaccine formulation of any one of claims 105-108, wherein the formulation is stable after at least three freeze/thaw cycles.

110. The vaccine formulation of any one of claims 105-109, wherein the formulation allows the recombinant virus particles of any one of claim 1-51 or 98 to remain viable after three freeze/thaw cycles.

111. The vaccine formulation of any one of claims 105-110, wherein the formulation increases the time the recombinant virus particles of any one of claim 1-51 or 98 is in contact with mucous membranes.

112. The composition of claim 97 or 100, the pharmaceutical composition of claim 101 or claim 102, the immunogenic composition of claim 103 or claim 104, or the vaccine formulation of any one of claims 105-111, wherein the composition or formulation comprises 50 mM Tris and 2 mM MgCl₂ and is at pH 7.4.

113. The composition of any one of claim 97, 100, or 112, the pharmaceutical composition of any one of claim 101, 102, or 112, the immunogenic composition of any one of claim 103, 104, or 112, or the vaccine formulation of any one of claims 105-112, wherein the carrier and/or excipient comprises at least one of methylcellulose, monosodium glutamate, human serum albumin, fetal bovine serum, trehalose, alginate, guar gum, or MUCOLOX™.

114. The vaccine formulation of any one of claims 105-113, wherein the formulation comprises 50 mM Tris HCL (pH 7.4), 2 mM MgCl₂, 10% Trehalose, and 0.25% Human Serum Albumin.

115. A method of treating or preventing a disease or disorder in a subject comprising administering to the subject an amount of the recombinant virus particle of any one of claim 1-51 or 98, the pharmaceutical composition of any one of claim 101, 102, 112, or 113, or the immunogenic composition of any one of claim 103, 104, 112, or 113, or the vaccine formulation of any one of claims 105-114.

116. A method of treating or preventing a disease or disorder in a subject comprising administering to the subject an amount of the recombinant virus particle of any one of claim 1-51 or 98, the pharmaceutical composition of any one of claim 101, 102, 112, or 113, or the immunogenic composition of any one of claim 103, 104, 112, or 113, or the vaccine formulation of any one of claims 105-114 effective to induce an immune response against a SARS-CoV-2.

117. A method of treating or preventing a disease or disorder in a subject comprising administering to the subject an amount of the recombinant virus particle of any one of claim 1-51 or 98, the pharmaceutical composition of any one of claim 101, 102, 112, or 113, the immunogenic composition of any one of claim 103, 104, 112, or 113, or the vaccine formulation of any one of claims 105-114 effective to induce the formation of neutralizing antibodies against a SARS-CoV-2.

118. A method of treating or preventing a disease or disorder in a subject comprising administering to the subject a boosting dose of the recombinant virus particle of any one of claim 1-51 or 98, the pharmaceutical composition of any one of claim 101, 102, 112, or 113, the immunogenic composition of any one of claim 103, 104, 112, or 113, or the vaccine formulation of any one of claims 105-114.

119. A method of treating or preventing a disease or disorder in a subject comprising administering to the subject a boosting dose of the recombinant virus particle of any one of claim 1-51 or 98, the pharmaceutical composition of any one of claim 101, 102, 112, or 113, the immunogenic composition of any one of claim 103, 104, 112, or 113, or the vaccine formulation of any one of claims 105-114 effective to induce an immune response against a SARS-CoV-2.

120. A method of treating or preventing a disease or disorder in a subject comprising administering to the subject a boosting of the recombinant virus particle of any one of claim 1-51 or 98, the pharmaceutical composition of any one of claim 101, 102, 112, or 113, the immunogenic composition of any one of claim 103, 104, 112, or 113, or the vaccine formulation of any one of claims 105-114 effective to induce the formation of neutralizing antibodies against a SARS-CoV-2.

121. The method of any one of claims 118-120, wherein the boosting dose is administered orally.

122. The method of any one of claims 115-121, wherein the disease or disorder is COVID-19.

123. A method of treating a subject infected with a SARS-CoV-2 comprising administering to the subject an amount of the recombinant virus particle of any one of claim 1-51 or 98, the pharmaceutical composition of any one of claim 101, 102, 112, or 113, or the immunogenic composition of any one of claim 103, 104, 112, or 113, or the vaccine formulation of any one of claims 105-114 effective to target the subject's cells harboring the SARS-CoV-2.

124. The method according to claims 115-123, wherein the subject is human.

125. A kit comprising an amount of the recombinant virus particle of any one of claim 1-51 or 98, the pharmaceutical composition of any one of claim 101, 102, 112, or 113, or the immunogenic composition of any one of claim 103, 104, 112, or 113, or the vaccine formulation of any one of claims 105-114 and, optionally, instructions.

126. A kit comprising an amount of the recombinant virus particle of any one of claim 1-51 or 98, the pharmaceutical composition of any one of claim 101, 102, 112, or 113, or the immunogenic composition of any one of claim 103, 104, 112, or 113, or the vaccine formulation of any one of claims 105-114 effective to induce an immune response against the SARS-CoV-2 and, optionally, instructions.

127. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

128. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

129. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 3 or an amino acid sequence that has at least 80% sequence identity to SEQ ID NO: 3, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

130. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 3 or an amino acid sequence that has at least 80% sequence identity to SEQ ID NO: 3, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

131. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a derivative of a SARS-CoV-2 spike (S) glycoprotein comprising the amino acid sequence of SEQ ID NO: 5 or an amino acid sequence that has at least 80% sequence identity to SEQ ID NO: 5, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

132. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a derivative of a SARS-CoV-2 spike (S) glycoprotein comprising the amino acid sequence of SEQ ID NO: 5 or an amino acid sequence that has at least 80% sequence identity to SEQ ID NO: 5, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

133. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

134. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a SARS-CoV-2 spike (S) glycoprotein comprising the amino acid sequence of SEQ ID NO: 1 or an amino acid sequence that has at least 80% sequence identity to SEQ ID NO: 1, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

135. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

136. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein polynucleotide.

137. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a fragment of a SARS-CoV-2 spike (S) glycoprotein comprising the polynucleotide sequence of SEQ ID NO: 4 or an polynucleotide sequence that has at least 80% sequence identity to SEQ ID NO: 4, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

138. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a fragment of a SARS-CoV-2 spike (S) glycoprotein comprising the polynucleotide sequence of SEQ ID NO: 4 or an polynucleotide sequence that has at least 80% sequence identity to SEQ ID NO: 4, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

139. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a derivative of a SARS-CoV-2 spike (S) glycoprotein comprising the polynucleotide sequence of SEQ ID NO: 6 or an polynucleotide sequence that has at least 80% sequence identity to SEQ ID NO: 6, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

140. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a derivative of a SARS-CoV-2 spike (S) glycoprotein comprising the polynucleotide sequence of SEQ ID NO: 6 or an polynucleotide sequence that has at least 80% sequence identity to SEQ ID NO: 6, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein derivative polynucleotide.

141. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

142. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a SARS-CoV-2 spike (S) glycoprotein comprising the polynucleotide sequence of SEQ ID NO: 2 or an polynucleotide sequence that has at least 80% sequence identity to SEQ ID NO: 2, wherein the polynucleotide sequence encoding the SARS-CoV-2 S glycoprotein replaces the VSV G gene and further comprises a Kozak sequence of SEQ ID NO: 11 3′ to the SARS-CoV-2 S glycoprotein polynucleotide.

143. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 20 or an amino acid sequence that has at least 80% sequence identity to SEQ ID NO: 20, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

144. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 20 or an amino acid sequence that has at least 80% sequence identity to SEQ ID NO: 20, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

145. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the polynucleotide sequence of SEQ ID NO: 10 or an polynucleotide sequence that has at least 80% sequence identity to SEQ ID NO: 10; and c) comprises a polynucleotide sequence encoding a fragment of a SARS-CoV-2 spike (S) glycoprotein comprising the polynucleotide sequence of SEQ ID NO: 21 or an polynucleotide sequence that has at least 80% sequence identity to SEQ ID NO: 21, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

146. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the polynucleotide sequence of SEQ ID NO: 8 or an polynucleotide sequence that has at least 80% sequence identity to SEQ ID NO: 8; and c) comprises a polynucleotide sequence encoding a fragment of a SARS-CoV-2 spike (S) glycoprotein comprising the polynucleotide sequence of SEQ ID NO: 21 or an polynucleotide sequence that has at least 80% sequence identity to SEQ ID NO: 21, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

147. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a wild-type VSV matrix (M) protein comprising the amino acid sequence of SEQ ID NO: 9 or an amino acid sequence that has at least 80% sequence identity to SEQ ID NO: 9; and c) comprises a polynucleotide sequence encoding a fragment of a SARS-CoV-2 spike (S) glycoprotein comprising or consisting of the amino acid sequence of SEQ ID NO: 22 or an amino acid sequence that has at least 80% sequence identity to SEQ ID NO: 22, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

148. A replication-competent recombinant vesicular stomatitis virus (VSV) particle, wherein the recombinant VSV particle comprises a VSV genome, wherein said VSV genome a) lacks a functional VSV glycoprotein G gene; b) comprises a polynucleotide encoding a mutant VSV matrix (M) protein comprising the amino acid sequence of SEQ ID NO: 7 or an amino acid sequence that has at least 80% sequence identity to SEQ ID NO: 7; and c) comprises a polynucleotide sequence encoding a fragment of a SARS-CoV-2 spike (S) glycoprotein comprising or consisting of the amino acid sequence of SEQ ID NO: 22 or an amino acid sequence that has at least 80% sequence identity to SEQ ID NO: 22, wherein the polynucleotide sequence encoding the SARS-CoV-2 S glycoprotein replaces the VSV G gene and further comprises an optimized Kozak sequence of SEQ ID NO: 12 3′ to the SARS-CoV-2 S glycoprotein fragment polynucleotide.

149. A recombinant virus particle, wherein the recombinant virus particle is a recombinant vesiculovirus particle comprising a vesiculovirus genome lacking a functional vesiculovirus glycoprotein G gene, and further wherein the recombinant virus particle comprises a polynucleotide sequence encoding at least one Severe Acute Respiratory Syndrome coronavirus 2 (SARS-CoV-2) spike (S) glycoprotein or fragment or derivative thereof.

150. The recombinant virus particle of claim 149, wherein the recombinant vesiculovirus particle further comprises a pseudotyped G glycoprotein or fragment or derivative that is derived from a rhabdovirus that is not the recombinant vesiculovirus.

151. The recombinant virus particle of claim 150, wherein the polynucleotide sequence encoding the SARS-CoV-2 S glycoprotein or fragment comprises one or more mutations.

152. The recombinant virus particle of any of claims 150 and 151, wherein the recombinant virus particle is a vaccine.

153. The recombinant virus particle of claim 152, wherein the vaccine is administered orally.

154. The recombinant virus particle of any of claims 152 and 153, wherein the vaccine is administered as a primary vaccination or a boost.


SEQUENCE LISTING (22 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Artificial Sequence
  source: /note=“Description of Artificial Sequence Synthetic polynucleotide”
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 3 - protein, 1254 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 4 - DNA, 3765 bp, Artificial Sequence
  source: /note=“Description of Artificial Sequence Synthetic polynucleotide”
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgttaa

SEQ ID NO: 5 - protein, 1258 aa, Artificial Sequence
  source: /note=“Description of Artificial Sequence Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMKLKHTKKRQIYTDIEMNRLGK

SEQ ID NO: 6 - DNA, 3777 bp, Artificial Sequence
  source: /note=“Description of Artificial Sequence Synthetic polynucleotide”
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgaaattaaagcacaccaagaaaagacagatttatacagacatagagatgaaccgacttggaaagtaa

SEQ ID NO: 7 - protein, 237 aa, Artificial Sequence
  source: /note=“Description of Artificial Sequence Synthetic polypeptide”
MSSLKKILGLKGKGKKSKKLGIAPPPYEEDTSMEYAPSAPIDKSYFGVDERDTHDPNQLRYEKSFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEEEASGAWVLDSVRHSKWASLASSF

SEQ ID NO: 8 - DNA, 690 bp, Artificial Sequence
  source: /note=“Description of Artificial Sequence Synthetic polynucleotide”
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagcgagacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 9 - protein, 237 aa, Vesicular stomatitis virus
MSSLKKILGLKGKGKKSKKLGIAPPPYEEDTSMEYAPSAPIDKSYFGVDEMDTHDPNQLRYEKSFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEEEASGAWVLDSVRHSKWASLASSF

SEQ ID NO: 10 - DNA, 690 bp, Vesicular stomatitis virus
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 11 - DNA, 7 bp, Vesicular stomatitis virus
cactatg

SEQ ID NO: 12 - DNA, 7 bp, Artificial Sequence
  source: /note=“Description of Artificial Sequence Synthetic oligonucleotide”
caccatg

SEQ ID NO: 13 - protein, 1185 aa, Severe acute respiratory syndrome coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 14 - protein, 19 aa, Severe acute respiratory syndrome coronavirus 2
KFDEDDSEPVLKGVKLHYT

SEQ ID NO: 15 - protein, 21 aa, Vesicular stomatitis virus
KLKHTKKRQIYTDIEMNRLGK

SEQ ID NO: 16 - protein, 316 aa, Artificial Sequence
  source: /note=“Description of Artificial Sequence Synthetic polypeptide”
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDESGGGGMSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADK

SEQ ID NO: 17 - protein, 236 aa, Artificial Sequence
  source: /note=“Description of Artificial Sequence Synthetic polypeptide”
MQKNGIKANFTVRHNVEDGSVQLADHYQQNTPIGDGPVLLPDNHYLSTQTVLSKDPNEKRDHMVLHEYVNAAGITGGGGSWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 18 - protein, 231 aa, Artificial Sequence
  source: /note=“Description of Artificial Sequence Synthetic polypeptide”
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKMQKNGIKANFTVRHNVEDGSVQLADHYQQNTPIGDGPVLLPDNHYLSTQTVLSKDPNEKRDHMVLHEYVNAAGIT

SEQ ID NO: 19 - protein, 311 aa, Artificial Sequence
  source: /note=“Description of Artificial Sequence Synthetic polypeptide”
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 20 - protein, 1254 aa, Artificial Sequence
  source: /note=“Description of Artificial Sequence Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRRYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEHVNNSYECDIPIGAGICASYQTQTNSPRRAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 21 - DNA, 3765 bp, Artificial Sequence
  source: /note=“Description of Artificial Sequence Synthetic polynucleotide”
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacattatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagatacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagaacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccagagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgttaa

SEQ ID NO: 22 - protein, 1252 aa, Artificial Sequence
  source: /note=“Description of Artificial Sequence Synthetic polypeptide”
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC